(19) United States
(12) Patent Application Publication
(10) Pub. No.: US 2025/0114548 A1
(43) Pub. Date: Apr. 10, 2025

(54) MEASUREMENT DEVICE AND SYSTEM FOR BREATHING ASSISTANCE APPARATUS AND/OR PERFORMING DIAGNOSTICS

(71) Applicant: Fisher & Paykel Healthcare Limited, Auckland (NZ)

(72) Inventors: William Randell Smith, Auckland (NZ); Charles Grady Cantrell, Auckland (NZ); David Martin Russell, Auckland (NZ); Anton Kim Gulley, Auckland (NZ); Madeleine Rose Clements, Auckland (NZ)

(21) Appl. No.: 18/716,365

(22) PCT Filed: Dec. 14, 2022
(86) PCT No.: PCT/IB2022/062245

§ 371 Date: Jun. 4, 2024

Related U.S. Application Data
(60) Provisional application No. 63/290,873, filed on Dec. 17, 2021.

(51) Int. Cl. A61M 16/00; A61B 5/091; A61M 16/10

(52) U.S. Cl. CPC A61M 16/024; A61B 5/091; A61M 16/0003; A61M 16/10

(57) ABSTRACT

A measurement device for use with a breathing assistance apparatus to measure lung performance. The device has a main body that is releasably connectable to a gases outlet or a breathing conduit of the breathing assistance apparatus. The main body conveys a flow of gases from the gases outlet to a user. The main conduit body has a connector end that is connectable to the gases outlet of the breathing assistance apparatus to receive the flow of gases, and a mouthpiece end to provide fluid communication to the user's airway. A main lumen or lumens extend between the connector end and mouthpiece end for conveying the flow of gases. One or more exhaust openings provided on the main body between its ends and which are in fluid communication with the main lumen or lumens.

### FIELD OF THE DISCLOSURE

The present disclosure relates to a measurement device and system for a breathing assistance apparatus and/or performing diagnostics. In particular, the present disclosure relates to a measurement device and/or system for undertaking lung performance or lung function diagnostics or measurements.

### BACKGROUND

Breathing assistance apparatuses are used in various environments such as hospital, medical facility, residential care, or home environments to deliver a flow of gases to users or patients. A breathing assistance or respiratory therapy apparatus may be used to deliver supplementary oxygen or other gases with a flow of gases, and/or a humidification apparatus to deliver heated and humidified gases. A breathing assistance apparatus may allow adjustment and control over characteristics of the gases flow, including flow rate, temperature, gases concentration, humidity, pressure, etc. Sensors, such as flow sensors and/or pressure sensors are used to measure characteristics of the gases flow.

### SUMMARY

The present disclosure relates to a measurement device and system for use with a breathing assistance apparatus to undertake lung performance or function diagnostics or measurements.

In one configuration, the measurement device may be used to perform spirometry measurements or may be used to obtain measurements that are indicative of, related to, or analogous to spirometry measurements. In this configuration, the measurement device may be considered to be a surrogate spirometer attachment.

In another configuration, the measurement device may be used to perform other measurements or diagnostics in relation to lung function or lung performance of a user.

In one configuration, the measurement device may be an auxiliary component or attachment that can be connected to the gases outlet of a breathing assistance apparatus or to the end of a breathing circuit conduit of or connected to a breathing assistance apparatus, such that the measurement device receives a flow of gases from the breathing assistance apparatus.

The present disclosure provides a measurement device and system for undertaking lung performance or function measurements with a breathing assistance apparatus.

In an aspect, this disclosure broadly comprises a breathing assistance apparatus that is configured to provide a flow of gases to a user for respiratory therapy comprising: a flow generator that is operable to generate a flow of gases along a flow path of the apparatus; a breathing conduit for delivery of the flow of gases to a user; a measurement device for lung performance that is fluidly connected or connectable to an end of the breathing conduit to receive the flow of gases; and a controller that is operable to control the breathing assistance apparatus, the controller being configured to control the flow of gases into the measurement device to provide a controlled pneumatic resistance to user exhaled breath as the user performs one or more forced expiratory manoeuvres into the measurement device.

In an aspect, this disclosure broadly comprises a breathing assistance apparatus that is configured to provide a flow of gases to a user for respiratory therapy comprising: a flow generator that is operable to generate a flow of gases along a flow path of the apparatus; a breathing conduit for delivery of the flow of gases to a user; a measurement device for lung performance that is fluidly connected or connectable to an end of the breathing conduit to receive the flow of gases; and a controller that is operable to control the breathing assistance apparatus, the controller being configured to control the flow of gases into the measurement device to provide a controlled pneumatic resistance to user exhaled breath as the user performs restful or tidal breathing into the measurement device.

In a configuration, the controller is configured to determine a user flow rate signal representing the flow the user is able to induce against the flow of gases via the measurement device based on a function of at least a user pressure signal representing the pressure exerted by the user during exhalation into the measurement device.

In a configuration, the controller is configured to determine a user flow rate signal representing the flow the user is able to induce against the flow of gases via the measurement device based on a function of at least a flow resistance parameter relating to the measurement device.

In a configuration, the controller is configured to determine a user flow rate signal representing the flow the user is able to induce against the flow of gases via the measurement device based on a function of at least a flow conductance parameter.

In a configuration, the controller is configured to determine the flow conductance parameter based on a function of at least a flow rate signal representing the flow rate of the flow of gases generated by the flow generator and the user pressure signal representing the pressure exerted by the user during exhalation into the measurement device.

In a configuration, the controller is configured to determine the user pressure signal based on a function of at least a pressure signal representing the pressure at the outlet of the flow generator, a resistance to flow parameter, and a flow rate signal representing the flow rate of the flow of gases generated by the flow generator.

In a configuration, the controller is configured to determine a user flow rate signal representing the flow the user is able to induce against the flow of gases via the measurement device based on a function of at least a flow rate signal representing the flow rate of the flow of gases generated by the flow generator and a leak flow signal representing the leak flow through one or more exhaust openings of the measurement device.

In a configuration, the controller is configured to determine the leak flow signal based on a function of at least a flow conductance parameter and a user pressure signal representing the pressure exerted by the user during exhalation into the measurement device.

In a configuration, the controller is configured to determine the user pressure signal based on a function of at least a pressure signal representing the pressure at the outlet of the flow generator, a resistance to flow parameter, and a flow rate signal representing the flow rate of the flow of gases generated by the flow generator.

In a configuration, the controller is configured to determine a respiratory rate signal or parameter for the user based at least partly on or as a function of at least the determined user flow rate signal, as the user performs tidal breathing into the measurement device.

In a configuration, the controller is configured to determine a tidal volume signal or parameter for the user based at least partly on or as a function of at least the determined user flow rate signal, as the user performs tidal breathing into the measurement device.

In a configuration, the controller is configured to determine a minute ventilation signal or parameter for the user based at least partly on or as a function of at least the determined respiratory rate signal and tidal volume signal.

In a configuration, the controller is configured to determine a signal or parameter representing the inspiratory time to total breath time ratio based at least partly on or as a function of at least the determined user flow rate signal, as the user performs tidal breathing into the measurement device.

In a configuration, the apparatus comprises one or more sensors for sensing or determining one or more characteristics of the flow of gases and generating representative sensor data.

In a configuration, the one or more sensors are configured to measure or determine any one or more of the following characteristics of the flow of gases: flow rate, pressure, temperature, humidity.

In a configuration, the one or more sensors are external or separate to the measurement device.

In a configuration, the one or more sensors are located in the flow path upstream of the measurement device or configured to sense the flow of gases upstream of the measurement device.

In a configuration, the apparatus the flow generator is enclosed or contained in a main housing of the apparatus, and wherein the one or more sensors are located within the main housing and/or breathing conduit of the apparatus.

In a configuration, the measurement device comprises a main body extending between a connector end and a mouthpiece end, the main body comprising a main lumen or lumens extending between the connector end and mouthpiece end for the flow of gases.

In a configuration, the main body is a conduit or tubular component with an opening or openings at each end.

In a configuration, the connector end of the main body is configured to be releasably connected to an end of the breathing conduit.

In a configuration, the connector end of the main body is integrally or permanently connected to an end of the breathing conduit.

In a configuration, the connector end of the main body comprises one or more openings or ports for fluidly connecting to an end of the breathing conduit.

In a configuration, the mouthpiece end of the main body is configured to receive a removable mouthpiece that is configured to provide fluid communication to the user's airway, when in use.

In a configuration, the mouthpiece end of the main body is a mouthpiece that is configured to provide fluid communication to the user's airway, when in use.

In a configuration, the main body of the measurement device further comprises one or more exhaust openings that are in fluid communication with the main lumen or lumens.

In a configuration, the measurement device further comprises one or more anti-occlusion features that are configured to prevent inadvertent blockage of the one or more exhaust openings.

In a configuration, the anti-occlusion features are arranged or configured as a clip for mounting the measurement device.

In a configuration, the one or more exhaust openings are provided on the main body between the connector end and mouthpiece end.

In a configuration, one or more of the exhaust openings are vents.

In a configuration, one or more of the vents comprises a single opening.

In a configuration, one or more of the vents comprises a plurality of openings.

In a configuration, one or more of the vents comprises multiple openings or holes in the form of a honeycomb or mesh structure or arrangement.

In a configuration, one or more of the exhaust openings are flush vents provided in a wall of the main body such that the flush vents are substantially flush with an outer surface of the main body.

In a configuration, one or more of the exhaust openings are protruding vents that protrude from a wall of the main body.

In a configuration, one or more of the protruding vents are shaped and/or configured to direct or divert any exhaust gases exiting the vent away from a user's face, when the measurement device is in use.

In a configuration, the main body is defined by a longitudinal axis extending between the connector end and mouthpiece end, and the one or more of the protruding vents extends at an angle relative to the longitudinal axis such that any exhaust gases exiting the vent are directed or diverted away from the user's face, when the measurement device is in use.

In a configuration, the apparatus further comprises a humidifier that is operable to heat and humidify the flow of gases generated by the flow generator.

In a configuration, the humidifier is provided in a main housing of the apparatus, the main housing further comprising the flow generator.

In a configuration, the controller comprises a patient diagnostic mode that is operable to control the flow of gases to provide the controlled pneumatic resistance when the measurement device is connected to an end of the breathing conduit.

In a configuration, the controller comprises one or more operating modes, including at least a first mode that is operable to control the apparatus when the measurement device is not connected to the breathing conduit and a second mode that is operable to control the apparatus when the measurement device is connected to the breathing conduit.

In a configuration, the controller comprises a plurality of operating modes, including at least a therapy mode that is operable to control the apparatus to provide the flow of gases to user for respiratory therapy, and a patient diagnosis mode that is operable to control the flow of gases to provide a pneumatic resistance for performing lung performance measurements when the measurement device is connected to the breathing conduit.

In an aspect, this disclosure broadly comprises an apparatus for performing lung performance measurements on a user, comprising: a flow generator that is operable to generate a flow of gases in a flow path; a measurement device that is in fluid communication with the flow path to receive the flow of gases; a controller that is operable to control the flow generator to generate a flow of gases into the measurement device to provide a controlled pneumatic resistance in the flow path to user exhaled breath as the user performs one or more forced expiratory manoeuvres into the measurement device; and one or more sensors that are configured to sense one or more characteristics of the flow of gases in the flow path as the user performs the forced expiratory manoeuvres into the measurement device and generates representative sensor data from which one or more lung performance measurements can be obtained.

In an aspect, this disclosure broadly comprises an apparatus for performing lung performance measurements on a user, comprising: a flow generator that is operable to generate a flow of gases in a flow path; a measurement device that is in fluid communication with the flow path to receive the flow of gases; a controller that is operable to control the flow generator to generate a flow of gases into the measurement device to provide a controlled pneumatic resistance in the flow path to user exhaled breath as the user performs restful or tidal breathing into the measurement device; and one or more sensors that are configured to sense one or more characteristics of the flow of gases in the flow path as the user performs the restful or tidal breathing into the measurement device and generates representative sensor data from which one or more lung performance measurements can be obtained.

In an aspect, this disclosure broadly comprises a measurement device for use with a breathing assistance apparatus to measure lung performance comprising: a main body that is releasably connectable to a gases outlet or a breathing conduit of the breathing assistance apparatus and which is configured to convey a flow of gases from the gases outlet to a user, and wherein the main conduit body comprises: a connector end that is connectable to the gases outlet of the breathing assistance apparatus to receive the flow of gases; a mouthpiece end to provide fluid communication to the user's airway, when in use; a main lumen or lumens extending between the connector end and mouthpiece end for conveying the flow of gases; and one or more exhaust openings provided on the main body between its ends and which are in fluid communication with the main lumen or lumens.

In a configuration, the main body is a conduit or tubular component with an opening or openings at each end.

In a configuration, the connector end of the main body is configured to be releasably connectable to the gases outlet or an end of the breathing conduit of the breathing assistance apparatus.

In a configuration, the connector end of the main body comprises one or more openings or ports for fluidly connecting to the gases outlet or the breathing conduit of the breathing assistance apparatus.

In a configuration, the mouthpiece end of the main body is configured to receive a removable mouthpiece that is configured to provide fluid communication to the user's airway, when in use.

In a configuration, the mouthpiece end of the main body is a mouthpiece that is configured to provide fluid communication to the user's airway, when in use.

In a configuration, the main body of the measurement device further comprises one or more exhaust openings that are in fluid communication with the main lumen or lumens.

In a configuration, the measurement device further comprises one or more anti-occlusion features that are configured to prevent inadvertent blockage of the one or more exhaust openings.

In a configuration, the anti-occlusion features are arranged or configured as a clip for mounting the measurement device.

In a configuration, the one or more exhaust openings are provided on the main body between the connector end and mouthpiece end.

In a configuration, one or more of the exhaust openings are vents.

In a configuration, one or more of the vents comprises a single opening.

In a configuration, one or more of the vents comprises a plurality of openings.

In a configuration, one or more of the vents comprises multiple openings or holes in the form of a honeycomb or mesh structure or arrangement.

In a configuration, one or more of the exhaust openings are flush vents provided in a wall of the main body such that the flush vents are substantially flush with an outer surface of the main body.

In a configuration, one or more of the exhaust openings are protruding vents that protrude from a wall of the main body.

In a configuration, one or more of the protruding vents are shaped and/or configured to direct or divert any exhaust gases exiting the vent away from a user's face, when the measurement device is in use.

In a configuration, the main body is defined by a longitudinal axis extending between the connector end and mouthpiece end, and the one or more of the protruding vents extends at an angle relative to the longitudinal axis such that any exhaust gases exiting the vent are directed or diverted away from the user's face, when the measurement device is in use.

In an aspect, this disclosure broadly comprises a method of measuring lung performance using a measurement device with a breathing assistance apparatus, comprising the steps of: connecting a measurement device to a gases outlet or breathing conduit or flow path of a breathing assistance apparatus such that the measurement device receives a flow of gases from a flow generator of the breathing assistance apparatus; operating the breathing assistance apparatus in a patient diagnostic mode that is configured to enable one or more lung performance measurements to be determined; instructing the user to perform one or more forced expiratory manoeuvres into the measurement device; and determining one or more characteristics of the flow of gases using one or more sensors during the forced expiratory manoeuvres.

In an aspect, this disclosure broadly comprises a method of measuring lung performance using a measurement device with a breathing assistance apparatus, comprising the steps of: connecting a measurement device to a gases outlet or breathing conduit or flow path of a breathing assistance apparatus such that the measurement device receives a flow of gases from a flow generator of the breathing assistance apparatus; operating the breathing assistance apparatus in a patient diagnostic mode that is configured to enable one or more lung performance measurements to be determined; instructing the user to perform restful or tidal breathing into the measurement device; and determining one or more characteristics of the flow of gases using one or more sensors during the restful or tidal breathing.

In a configuration, the patient diagnostic mode of the breathing assistance apparatus is configured to generate a flow of gases to the gases outlet and/or breathing conduit connected to the measurement device.

In a configuration, the patient diagnostic mode of the breathing assistance apparatus is configured to generate the flow of gases at a constant flow rate.

In a configuration, the constant flow rate of the flow of gases is configured to provide a substantially constant or consistent pneumatic resistance to user exhaled breath into the measurement device.

In a configuration, the patient diagnostic mode of the breathing assistance apparatus is configured to generate the flow of gases for a fixed duration or predetermined time period.

In a configuration, operating the breathing assistance apparatus in the patient diagnostic mode comprises initiating or activating the patient diagnostic mode in response to user input via a user interface.

In a configuration, the user input to initiate or activate the patient diagnostic mode is provided via a user interface of the breathing assistance apparatus.

In a configuration, the user interface of the breathing assistance apparatus comprises a button and/or touch-screen interface button that is operable by a user or other person to activate the patient diagnostic mode.

In a configuration, user input to initiate or activate the patient diagnostic mode is provided via a user interface of an external device that is in data communication with the breathing assistance apparatus.

In a configuration, the user interface of the device comprises a button and/or a touch-screen interface button that is operable by a user or other person to activate the patient diagnostic mode.

In a configuration, the method further comprises prompting a user or other person to initiate or activate the patient diagnostic mode via the user interface.

In a configuration, prompting a user or other person to initiate or activate the patient diagnostic mode comprises providing the prompt via the breathing assistance apparatus.

In a configuration, prompting comprises generating an audio prompt by the breathing assistance apparatus.

In a configuration, prompting comprises generating a visual prompt on a display or interface of the breathing assistance apparatus.

In a configuration, prompting a user or other person to initiate or activate the patient diagnostic mode comprises providing the prompt on an external device that is in data communication with the breathing assistance apparatus.

In a configuration, prompting a user or other person to initiate or activate the patient diagnostic mode comprises prompting according to a periodic interval.

In a configuration, the periodic interval is configurable.

In a configuration, the method comprises configuring the periodic interval of the prompting via a user interface of the breathing assistance apparatus.

In a configuration, the method comprises configuring the periodic interval of the prompting via a user interface or software application of an external device in data communication with the breathing assistance apparatus.

In a configuration, the periodic interval of the prompting is remotely configurable by a clinician, physician or health practitioner using the external device.

In a configuration, prompting a user or other person to initiate or activate the patient diagnostic mode comprises initiating or triggering a prompt in response to input or interaction by a clinician, physician or health practitioner with a remote or external device that is in data communication with the breathing assistance apparatus.

In a configuration, initiating or activating the patient diagnostic mode of the breathing assistance apparatus automatically.

In a configuration, initiating or activating the patient diagnostic mode of the breathing assistance apparatus automatically based on a periodic interval.

In a configuration, the periodic interval for automatically initiating or activating the patient diagnostic mode is configurable.

In a configuration, the method comprises configuring the periodic interval of the patient diagnostic mode via a user interface of the breathing assistance apparatus.

In a configuration, the method comprises configuring the periodic interval of the patient diagnostic mode via a user interface or software application of an external device in data communication with the breathing assistance apparatus.

In a configuration, the periodic interval of the patient diagnostic mode is remotely configurable by a clinician, physician or health practitioner using the external device.

In a configuration, the breathing assistance apparatus comprises one or more communication modules that are configured to provide data communication between one or more external or remote devices.

In a configuration, determining one or more characteristics of the flow of gases during the forced expiratory manoeuvres or tidal breathing comprises using one or more sensors of the breathing assistance apparatus.

In a configuration, determining one or more characteristics of the flow of gases during the forced expiratory manoeuvres or tidal breathing comprises using one or more sensors in the flow path upstream of the measurement device or configured to sense the flow of gases upstream of the measurement device.

In a configuration, the method further comprises providing instructions to a user via a user interface of the breathing assistance apparatus about how to connect the measurement device to the apparatus, use the measurement device, and/or perform the forced expiratory manoeuvres or tidal breathing into the mouthpiece during a diagnostic session.

In a configuration, the instructions are provided or presented visually on a display of the user interface and/or audibly.

In a configuration, the method further comprises presenting sensor data from the one or more sensors or one or more lung performance measurements generated from the sensor data graphically on a display of a user interface of the breathing assistance apparatus.

In a configuration, the method further comprises processing sensor data from the one or more sensors to generate lung performance measurement data and/or one or more representative graphs or the sensor data and/or lung performance measurement data.

In a configuration, the method further comprises processing the lung performance measurement data and/or graphs to identify or determine the health status of a user.

In an aspect, this disclosure broadly comprises a non-transitory computer-readable medium having stored thereon computer executable instructions that, when executed on a processing device or devices, cause the processing device or devices to perform any method of any one or more aspects above.

In an aspect, this disclosure broadly comprises a set of application program interfaces or an application program interface (API) embodied on a computer-readable medium for execution on a processing device in conjunction with an application program that perform any method of any one or more aspects above.

Any aspect of this disclosure above may further comprise any one or more aspects or features mentioned in respect of any one or more of the other aspects.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 shows schematically a breathing assistance apparatus configured to provide a respiratory therapy to a patient.

FIG. 2 is a front view of an example breathing assistance apparatus with a humidification chamber in position and a raised handle/lever.

FIG. 3 is a top view corresponding to FIG. 2.

FIG. 4 is a right side view corresponding to FIG. 2.

FIG. 5 is a left side view corresponding to FIG. 2.

FIG. 6 is a rear view corresponding to FIG. 2.

FIG. 7 is a front left perspective view corresponding to FIG. 2.

FIG. 8 is a front right perspective view corresponding to FIG. 2.

FIG. 9 is a bottom view corresponding to FIG. 2.

FIG. 10 shows an example configuration of an air and oxygen inlet arrangement of a breathing assistance apparatus.

FIG. 11 shows another example configuration of an air and oxygen inlet arrangement of the breathing assistance apparatus.

FIG. 12 is a transverse sectional view showing further detail of the air and oxygen inlet arrangement of FIG. 11.

FIG. 13 is another transverse sectional view showing further detail of the air and oxygen inlet arrangement of FIG. 11.

FIG. 14 is a longitudinal sectional view showing further detail of the air and oxygen inlet arrangement of FIG. 11.

FIG. 15 is an exploded view of upper and lower chassis components of a main housing of the breathing assistance apparatus.

FIG. 16 is a front left side perspective view of the lower chassis of the main housing showing a housing for receipt of a motor/sensor module sub-assembly.

FIG. 17 is a first underside perspective view of the main housing of the breathing assistance apparatus showing a recess inside the housing for the motor/sensor module sub-assembly.

FIG. 18 is a second underside perspective view of the main housing of the breathing assistance apparatus showing the recess for the motor/sensor module sub-assembly.

FIG. 19A illustrates a block diagram of a control system interacting with and/or providing control and direction to components of a breathing assistance apparatus.

FIG. 19B illustrates a block diagram of an example controller.

FIG. 20 illustrates a block diagram of a motor and sensor module.

FIG. 21 illustrates a sensing chamber of an example motor and sensor module.

FIG. 22 shows a schematic diagram of the breathing assistance apparatus of FIG. 1 with an embodiment of a measurement device connected to the end of the breathing conduit for measuring lung performance in a patient diagnostic mode of the apparatus in an embodiment.

FIG. 23 shows a perspective view from a connector end of a measurement device in a first example embodiment.

FIG. 24 shows a perspective view from a mouthpiece end of the measurement device of FIG. 23.

FIG. 25 shows a first side elevation view of the measurement device of FIG. 23.

FIG. 26 shows a second side elevation view of the measurement device of FIG. 23.

FIG. 27 shows an underside view of the measurement device of FIG. 23.

FIG. 28 shows a plan view of the measurement device of FIG. 23.

FIG. 29 shows a first end view of the measurement device of FIG. 23 from a mouthpiece end.

FIG. 30 shows a second end view of the measurement device of FIG. 23 from a connector end.

FIG. 31 shows a cross-sectional view of the measurement device through line AA of FIGS. 29 and 30.

FIG. 32 shows a close-up perspective view of a connector end of the measurement device of FIG. 23.

FIG. 33 shows a first end perspective view of a first example embodiment of a removable mouthpiece for the measurement device.

FIG. 34 shows a second end perspective view of the removable mouthpiece of FIG. 33.

FIG. 35 shows a side elevation view of the removable mouthpiece of FIG. 33.

FIG. 36 shows a cross-sectional view of the removable mouthpiece through line DD of FIG. 35

FIG. 37 shows a perspective view of the measurement device of FIG. 23 assembled with the removable mouthpiece of FIG. 33 in an embodiment.

FIG. 38 shows an exploded perspective view of the measurement device and removable mouthpiece of FIG. 37.

FIG. 39 shows a side view of the measurement device with assembled mouthpiece of FIG. 37.

FIG. 40 shows a cross-sectional view of the measurement device with assembled mouthpiece through line EE of FIG. 39.

FIG. 41 shows a side cross-sectional view of the measurement device with the assembled mouthpiece of FIG. 39.

FIG. 42 is a flow diagram of the process of performing lung performance measurements with the measurement device and a breathing assistance apparatus in accordance with one example configuration.

FIG. 43 is a flow diagram of prompts instructing a user to perform forced expiratory manoeuvres in accordance with one example configuration.

FIG. 44 is a flow diagram of the process of performing lung performance measurements with the measurement device and a breathing assistance apparatus in accordance with another example configuration.

FIGS. 45A-45C show example schematic GUI display screen prompts instructing the user to disconnect the patient interface in accordance with an example configuration.

FIGS. 46A-46C show example schematic GUI display screen prompts instructing the user to connect the measurement device attachment to the flow path of the breathing assistance apparatus.

FIGS. 47A-47F show example schematic GUI display screen prompts instructing the user to perform forced exhalation manoeuvres in accordance with an example configuration.

FIGS. 48A and 48B show example schematic GUI display screen prompts instructing the user to breath normally following the forced exhalation manoeuvres in accordance with an example configuration.

FIG. 49 shows an example schematic GUI display screen prompt instructing the user to connect a patient interface to the apparatus for a therapy session.

FIG. 50 is a graph of example sensed flow rate data for healthy and sick persons performing lung performance measurements with the measurement device and breathing assistance apparatus.

FIG. 51 shows a perspective view from a connector end of a measurement device in a second example embodiment.

FIG. 52 shows a perspective view from a mouthpiece end of the measurement device of FIG. 51.

FIG. 53 shows a first side elevation view of the measurement device of FIG. 51.

FIG. 54 shows a second side elevation view of the measurement device of FIG. 51.

FIG. 55 shows a plan view of the measurement device of FIG. 51.

FIG. 56 shows an underside view of the measurement device of FIG. 51.

FIG. 57 shows a first end view of the measurement device of FIG. 51 from a mouthpiece end.

FIG. 58 shows a second end view of the measurement device of FIG. 51 from a connector end.

FIG. 59 shows a cross-sectional view of the measurement device through lines AA of FIGS. 57 and 58.

FIG. 60 shows a close-up perspective view of a connector end of the measurement device of FIG. 51.

FIG. 61 shows a first end perspective view of a second example embodiment of a removable mouthpiece for the measurement device.

FIG. 62 shows a second end perspective view of the removable mouthpiece of FIG. 61.

FIG. 63 shows a plan view of the removable mouthpiece of FIG. 61.

FIG. 64 shows a cross-sectional view of the removable mouthpiece through line BB of FIG. 63.

FIG. 65 shows a side elevation view of the removable mouthpiece of FIG. 61.

FIG. 66 shows a cross-sectional view of the removable mouthpiece through line CC of FIG. 65.

FIG. 67 shows a perspective view of the measurement device of FIG. 51 assembled with the removable mouthpiece of FIG. 61 in an embodiment.

FIG. 68 shows an exploded perspective view of the measurement device and removable mouthpiece of FIG. 67.

FIG. 69 shows a side view of the measurement device with assembled mouthpiece of FIG. 67.

FIG. 70 shows a cross-sectional view of the measurement device with assembled mouthpiece through line DD of FIG. 69.

FIG. 71 shows a plan view of the measurement device with assembled mouthpiece of FIG. 67.

FIG. 72 shows a cross-sectional view of the measurement device with assembled mouthpiece through line EE of FIG. 71.

FIG. 73 shows a first perspective view of the measurement device with assembled mouthpiece of FIG. 67 including clip formations.

FIG. 74 shows a second perspective view of the measurement device with assembled mouthpiece and clip formations of FIG. 73.

FIG. 75 shows a side elevation view of the measurement device with assembled mouthpiece and clip formations of FIG. 73.

### DETAILED DESCRIPTION

Although certain examples are described below, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described below.

### 1. Overview of Measurement Device and System

The measurement device is an attachment or auxiliary component for use with a breathing assistance apparatus to undertake lung performance or lung function measurements and/or diagnostic measurements generally. The measurement device attachment is configured to fluidly connect or couple to the flow of gases generated by a breathing assistance apparatus, or is otherwise in fluid communication with the flow of gases. For example, in one configuration, the measurement device attachment is configured to attach or connect to the end of, or partway along, the flow path of a breathing assistance apparatus such that it receives a flow of gases generated by the breathing assistance apparatus.

In one configuration, the measurement device may attach or connect to the end of a patient breathing conduit, such as a flexible breathing conduit or tube connected to the gases outlet of the apparatus.

In another configuration, the measurement device may attach or connect directly to the gases outlet or any other accessible outlet or port along the flow path of the breathing assistance apparatus. In one example, the measurement device may attach to the gases outlet or patient outlet port of the apparatus that typically couples or connects with a patient breathing conduit. In another example, the breathing assistance apparatus may comprise a removable humidification chamber that is in fluid communication with the flow generator such that it receives the flow of gases generated by the flow generator.

The removable humidification chamber may be connected or fluidly coupled to the flow generator by a gases outlet port of the apparatus. The gases outlet port may be the outlet of the flow generator or may be in fluid communication with the flow generator outlet. When the humidification chamber is removed, the gases outlet port is accessible, and the measurement device may directly attach to the gases outlet port to receive the flow of gases generated by the flow generator. In other configurations, the humidifier or humidifier chamber may be bypassed via a bypass conduit or other bypass configuration. For example, the measurement device may attach directly to or indirectly via the breathing conduit to a bypass conduit or port or outlet that bypasses the humidifier or humidifier chamber, such that the measurement device is in fluid communication with the flow of gases from the flow generator outlet, and the humidifier is temporarily cut-out or bypassed from the flow path.

In any of the above configurations and examples, the measurement device connects or attaches to the flow path such that it is in fluid communication with the flow of gases generated by the flow generator and/or breathing assistance apparatus.

In an embodiment, the measurement device may comprise a main body extending between a connector end and a mouthpiece end. The connector and mouth-piece ends are fluidly connected by a main lumen or lumens (e.g. a channel or passage) extending between the ends of the main body. In one configuration, the main body may be a conduit, tubular or manifold component extending between the connector end and mouthpiece end. The main body may be substantially hollow. The connector end comprises one or more openings or ports for fluidly connecting to an end of a flexible breathing conduit of the breathing assistance apparatus. The mouthpiece end provides or receives a mouthpiece that comprises one or more openings or ports to provide fluid communication with the user's airway, when in use. One or more exhaust openings (e.g. vents) to atmosphere or the surrounding environment may also be provided along the main body between the connector and mouth-piece ends. The vent or vents may be in fluid communication with the main lumen.

In one embodiment, when the measurement device is attached or connected to the end of the flow path for use, a controller of the breathing assistance apparatus may switch, manually or automatically, into a diagnosis or measurement mode. In this diagnosis or measurement mode, the controller may prompt the user (e.g. via visual and/or audible prompts or notifications) to undertake or follow one or more steps to perform one or more lung function or lung performance measurements or tests. Typically, the controller prompts the user to perform forced expiratory manoeuvres, such as forcibly exhaling into the mouthpiece. The tests may include spirometry-type tests or manoeuvres.

During the measurements or tests, the controller may control the flow generator of the breathing assistance apparatus to provide a flow of gases (e.g. air or air supplemented with one or more other gases) according to a configurable flow rate setting. The generated flow of gases provides, creates and/or generates a pneumatic resistance or airflow resistance in the flow path against the user's exhalation manoeuvres. During the measurement or testing process, the breathing assistance apparatus senses one or more characteristics of the flow of gases in the flow path via one or more sensors. The sensor data may comprise signals, data or artefacts indicative of the user's exhaled breath during the test process. The sensors may be provided in the main housing of the breathing assistance apparatus and/or in the patient breathing conduit. The sensor data may be stored and analysed to generate output data representing lung performance or lung function. The output data may include spirometry data or data indicative of or analogous to spirometry measurements in some configurations.

In the following description, an example breathing assistance apparatus will be described, which can be used with the measurement device. The example breathing assistance apparatus is configured primarily for high flow therapy or which has a high flow therapy mode. However, the measurement device can be used with any breathing assistance apparatus, system or device having a flow generator (e.g. blower, fan, compressor or the like) that is operable to generate a controllable flow of gases for respiratory therapy or therapies. The breathing assistance apparatus may be operable or configured to provide a single type of therapy or may be operable to provide a plurality or multiple respiratory therapies. For example, the breathing assistance apparatus may be configured to operate in a single therapy mode, or may be configured to operate in or provide a plurality or multiple selectable therapy modes. By way of example, the measurement device may operate with a breathing assistance apparatus that provides any one or more of the following respiratory therapies and/or therapy modes: high flow therapy, Positive Airway

Pressure (PAP) therapy, Continuous Positive Airway Pressure (CPAP) therapy, Non-invasive ventilation (NIV) therapy, Bi-Level PAP or Bi-Level therapy, or other such respiratory therapies. The measurement device may operate with a breathing assistance apparatus or system comprising an active or passive humidifier in the flow path, or an apparatus or system without a humidifier.

### 2. Overview of Example Breathing Assistance Apparatus

The measurement device, and methods and processes of using the measurement device, will be described in the context of an example breathing assistance apparatus 10 that is configured or operable to provide nasal high flow therapy via a unsealed patient interface. This is intended as a non-limiting example. It will be appreciated that the measurement device may be operable with a broad range of breathing assistance apparatus comprising flow generators.

### 2.1 Schematic Overview

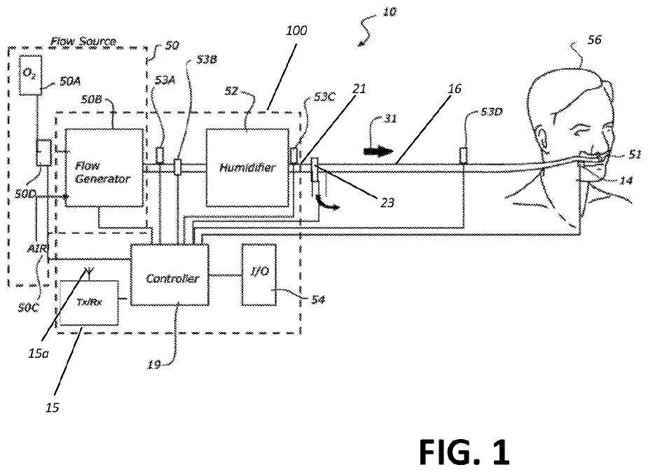

A schematic representation of the example breathing assistance apparatus 10 is provided in FIG. 1.

The breathing assistance apparatus 10 (or ‘respiratory system’) comprises a flow source 50 for providing a high flow gas 31 such as air, oxygen, air blended with oxygen, or a mix of air and/or oxygen and one or more other gases. Alternatively, the breathing assistance apparatus can have a connection for coupling to a flow source. As such, the flow source might be considered to form part of the apparatus or be separate to it, depending on context, or even part of the flow source forms part of the apparatus, and part of the flow source falls outside of the apparatus. In short, depending on the configuration (some components may be optional), the system can include a combination of components selected from the following:
a flow source
humidifier for humidifying the gas-flow,
conduit (e.g. dry line or heated breathing tube),
patient interface,
non-return valve
filter

The apparatus or system will be described in more detail.

The flow source could be an in-wall supply of oxygen, a tank of oxygen 50A, a tank of other gas and/or a high flow apparatus with a flow generator 50B. FIG. 1 shows a flow source 50 with a flow generator 50B, with an optional air inlet 50C and optional connection to an O2 source (such as tank or O2 generator) 50A via a shut off valve and/or regulator and/or other gas flow control 50D, but this is just one option. The flow generator 50B can control flows delivered to the patient 56 using one or more valve, or optionally the flow generator 50B can comprise a blower. The flow source could be one or a combination of a flow generator 50B, 02 source 50A, air source 50C as described. The flow source 50 is shown as part of the apparatus 10, although in the case of an external oxygen tank or in-wall source, it may be considered a separate component, in which case the apparatus has a connection port to connect to such flow source. The flow source provides a (preferably high) flow of gas that can be delivered to a patient via a delivery conduit 16, and patient interface 51.

The patient interface 51 may be an unsealed (non-sealing) interface (for example when used in high flow therapy) such as a non-sealing nasal cannula, or a sealed (sealing) interface (for example when used in CPAP) such as a nasal mask, full face mask, or nasal pillows. In some embodiments, the patient interface 51 is a non-sealing patient interface which would for example help to prevent barotrauma (e.g. tissue damage to the lungs or other organs of the respiratory system due to difference in pressure relative to the atmosphere). In some embodiments, the patient interface 51 is a sealing mask that seals with the patient's nose and/or mouth. The patient interface may be a nasal cannula with a manifold and nasal prongs, and/or a face mask, and/or a nasal pillows mask, and/or a nasal mask, and/or a tracheostomy interface, or any other suitable type of patient interface. The flow source could provide a base gas flow rate of between, e.g. 0.5 litres/min and 375 litres/min, or any range within that range, or even ranges with higher or lower limits. Details of the ranges and nature of flow rates will be described later.

A humidifier 52 can optionally be provided between the flow source 50 and the patient to provide humidification of the delivered gas. One or more sensors 53A, 53B, 53C, 53D such as flow, oxygen fraction, pressure, humidity, temperature or other sensors can be placed throughout the system and/or at, on or near the patient 56. Alternatively, or additionally, sensors from which such parameters can be derived could be used. In addition, or alternatively, the sensors 53A-53D can be one or more physiological sensors for sensing patient physiological parameters such as, heart rate, oxygen saturation, partial pressure of oxygen in the blood, respiratory rate, partial pressure of CO2 in the blood. Alternatively, or additionally, sensors from which such parameters can be derived could be used. Other patient sensors could comprise EEG sensors, torso bands to detect breathing, and any other suitable sensors. In some configurations the humidifier may be optional, or it may be preferred due to the advantages of humidified gases helping to maintain the condition of the airways. One or more of the sensors might form part of the apparatus, or be external thereto, with the apparatus having inputs for any external sensors. The sensors can be coupled to or send their output to a controller 19.

A sensor 14 may be provided for measuring the oxygen fraction of air the patient inspires. This can be placed on the patient interface 51, for example, to measure or otherwise determine the fraction of oxygen proximate (at/near/close to) the patient's mouth and/or nose. The output from the sensor 14 is sent to a controller 19 to assist control of the breathing assistance apparatus to determine if the peak inspiratory demand is being met, and alter operation accordingly. The controller 19 is coupled to the flow source 50, humidifier 52 and sensor 14. It controls these and other aspects of the apparatus or system to be described below. The controller can operate the flow source to provide the delivered flow of gas at a desired flow rate high enough to meet peak inspiratory demand. In an alternative, the sensor 14 might convey measurements of oxygen fraction at the patient mouth and/or nose to a user, who then inputs the information to the respiratory apparatus/controller. Any disclosure/embodiment hereinafter could be read as having that alternative, where appropriate.

An optional non-return valve 23 may be provided in the breathing conduit 16. A filter or filters may be provided at the air inlet 50C and/or inlets to the flow generator 50B to filter the incoming gases before they are pressurized into a high flow gas 31 by to the flow generator 50B.

The breathing assistance apparatus 10 could be an integrated or a separate component-based arrangement, generally shown in the dotted box 100 in FIG. 1. In some configurations, the apparatus or system could be a modular arrangement of components. Furthermore, the apparatus or system may just comprise some of the components shown, not necessarily all are essential. Also, the conduit and patient interface do not have to be part of the system, and could be considered separate. Hereinafter it will be referred to as a breathing assistance apparatus or respiratory system, but this should not be considered limiting. Breathing assistance apparatus and respiratory system will be broadly considered herein to comprise anything that provides a flow rate of gas to a patient. Some such apparatus and systems include a detection system that can be used to determine if the flow rate of gas meets inspiratory demand.

The breathing assistance apparatus 10 can include a main device housing 100. The main device housing 100 can contain the flow generator 50B that can be in the form of a motor/impeller arrangement, an optional humidifier 52, a controller 19, and an input/output I/O user interface 54. The user interface 54 can include a display and input device(s) such as button(s), a touch screen (e.g. an LCD screen), a combination of a touch screen and button(s), or the like. The controller 19 can include one or more hardware and/or software processors and can be configured or programmed to control the components of the system, including but not limited to operating the flow generator 50B to create a flow of gases for delivery to a patient, operating the humidifier 52 to humidify and/or heat the gases flow, receiving user input from the user interface 54 for reconfiguration and/or user-defined operation of the breathing assistance apparatus 10, and outputting information (for example on the display) to the user. The user can be a patient, healthcare professional, or others.

With continued reference to FIG. 1, a patient breathing conduit 16 can be coupled to a gases flow outlet (gases outlet or patient outlet port) 21 in the main device housing 100 of the breathing assistance apparatus 10, and be coupled to a patient interface 17, such as a non-sealing interface like a nasal cannula with a manifold and nasal prongs. The patient breathing conduit 16 can also be a tracheostomy interface, or other unsealed interfaces.

The gases flow can be generated by the flow generator 50B, and may be humidified, before being delivered to the patient via the patient breathing conduit 16 through the patient interface 51. The controller 19 can control the flow generator 50B to generate a gases flow of a desired flow rate, and/or one or more valves to control mixing of air and oxygen or other breathable gas. The controller 19 can control a heating element in or associated with the humidification chamber 12, to heat the gases to a desired temperature that achieves a desired level of temperature and/or humidity for delivery to the patient. The patient breathing conduit 16 can have a heating element, such as a heater wire, to heat gases flow passing through to the patient. The heating element can also be under the control of the controller 19.

The humidifier 52 of the apparatus is configured to combine or introduce humidity with or into the gases flow. Various humidifier 52 configurations may be employed. In one configuration, the humidifier 52 can comprise a humidification chamber that is removable. For example, the humidification chamber may be partially or entirely removed or disconnected from the flow path and/or apparatus. By way of example, the humidification chamber may be removed for refilling, cleaning, replacement and/or repair for example. In one configuration, the humidification chamber may be received and retained by or within a humidification compartment or bay of the apparatus, or may otherwise couple onto or within the housing of the apparatus.

The humidification chamber of the humidifier 52 may comprise a gases inlet and a gases outlet to enable connection into the gases flow path of the apparatus. For example, the flow of gases from the flow generator 50B is received into the humidification chamber via its gases inlet and exits the chamber via its gases outlet, after being heated and/or humidified.

The humidification chamber contains a volume of liquid, typically water or similar. In operation, the liquid in the humidification chamber is controllably heated by one or more heaters or heating elements associated with the chamber to generate water vapour or steam to increase the humidity of the gases flowing through the chamber.

In one configuration, the humidifier is a Passover humidifier. In another configuration, the humidifier may be a non-Passover humidifier.

In one configuration, the humidifier may comprise a heater plate, for example associated or within a humidification bay that the chamber sits on for heating. The chamber may be provided with a heat transfer surface, e.g a metal insert, plate or similar, in the base or other surface of the chamber that interfaces or engages with the heater plate of the humidifier.

In another configuration, the humidification chamber may comprise an internal heater or heater elements inside or within the chamber. The internal heater or heater elements may be integrally mounted or provided inside the chamber, or may be removable from the chamber.

The humidification chamber may be any suitable shape and/or size. The location, number, size, and/or shape of the gases inlet and gases outlet of the chamber may be varied as required. In one configuration, the humidification chamber may have a base surface, one or more side walls extending up from the base surface, and an upper or top surface. In one configuration, the gases inlet and gases outlet may be position on the same side of the chamber. In another configuration, the gases inlet and gases outlet may be on different surfaces of the chamber, such as on opposite sides or locations, or other different locations.

In some configurations, the gases inlet and gases outlet may have parallel flow axes. In some configurations, the gases inlet and gases outlet may be positioned at the same height on the chamber.

The system 10 can use ultrasonic transducer(s), flow sensor(s) such as a thermistor flow sensor, pressure sensor(s), temperature sensor(s), humidity sensor(s), or other sensors, in communication with the controller 19, to monitor characteristics of the gases flow and/or operate the system 10 in a manner that provides suitable therapy. The gases flow characteristics can include gases concentration, flow rate, pressure, temperature, humidity, or others. The sensors 53A, 53B, 53C, 53D, 14, such as pressure, temperature, humidity, and/or flow sensors, can be placed in various locations in the main device housing 100, the patient conduit 16, and/or the patient interface 51. The controller 19 can receive output from the sensors to assist it in operating the breathing assistance apparatus 10 in a manner that provides suitable therapy, such as to determine a suitable target temperature, flow rate, and/or pressure of the gases flow. Providing suitable therapy can include meeting a patient's inspiratory demand. In the illustrated embodiment sensors 53A, 53B, and 53C are positioned in the housing of the apparatus, sensor 53D in the patient conduit 16, and sensor 14 in the patient interface 51.

The apparatus 10 can include one or more communication modules to enable data communication or connection with one or more external devices or servers over a data or communication link or data network, whether wired, wireless, or a combination thereof. In one configuration for example, the apparatus 10 can include a wireless data transmitter and/or receiver, or a transceiver 15 to enable the controller 19 to receive data signals in a wireless manner from the operation sensors and/or to control the various components of the system 10. The transceiver 15 or data transmitter and/or receiver module may have an antenna 15a as shown. In one example, the transceiver may comprise a Wi-Fi modem. Additionally, or alternatively, the data transmitter and/or receiver 15 can deliver data to a remote server or enable remote control of the system 10. The system 10 can include a wired connection, for example, using cables or wires, to enable the controller 19 to receive data signals 8 from the operation sensors and/or to control the various components of the apparatus 10. The apparatus 10 may comprise one or more wireless communication modules. For example, the apparatus may comprise a cellular communication module such as for example a 3G, 4G or 5G module. The module 15 may be or may comprise a modem that enables the apparatus to communicate with a remote server using an appropriate communication network. The communication may be two-way communication between the apparatus and a server or other remote system. The apparatus 10 may also comprise other wireless communication modules such as for example a Bluetooth module and/or a Wi-Fi module. The Bluetooth and/or WiFi module allow the apparatus to wirelessly send information to another device such as for example a smartphone or tablet or operate over a LAN (local area network) or Wireless LAN (WLAN). The apparatus may additionally, or alternatively, comprise a Near Field Communication (NFC) module to allow for data transfer and/or data communication.

The breathing assistance apparatus 10 may comprise a high flow therapy apparatus. High flow therapy as discussed herein is intended to be given its typical ordinary meaning, as understood by a person of skill in the art, which generally refers to a respiratory system delivering a targeted flow of humidified respiratory gases via an intentionally unsealed patient interface with flow rates generally intended to meet or exceed inspiratory flow of a user. Typical patient interfaces include, but are not limited to, a nasal or tracheal patient interface. Typical flow rates for adults often range from, but are not limited to, about fifteen litres per minute to about sixty litres per minute or greater. Typical flow rates for pediatric users (such as neonates, infants and children) often range from, but are not limited to, about one litre per minute per kilogram of user weight to about three litres per minute per kilogram of user weight or greater.

High flow therapy can also optionally include gas mixture compositions including supplemental oxygen and/or administration of therapeutic medicaments.

High flow therapy is often referred to as nasal high flow (NHF), humidified high flow nasal cannula (HHFNC), high flow nasal oxygen (HFNO), high flow therapy (HFT), or tracheal high flow (THF), among other common names. For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing the work of breathing. Additionally, high flow therapy may generate a flushing effect in the nasopharynx such that the anatomical dead space of the upper airways is flushed by the high incoming gases flow. The flushing effect can create a reservoir of fresh gas available of each and every breath, while minimizing re-breathing of carbon dioxide, nitrogen, etc. High flow therapy can also increase expiratory time of the patient due to pressure during expiration. This in turn reduces the respiratory rate of the patient.

The patient interface for use in a high flow therapy can be a non-sealing interface to prevent barotrauma, which can include tissue damage to the lungs or other organs of the patient's respiratory system due to difference in pressure relative to the atmosphere. The patient interface can be a nasal cannula with a manifold and nasal prongs, and/or an unsealed tracheostomy interface, or any other suitable type of patient interface.

### 2.2 Mechanical and Electronic Aspects

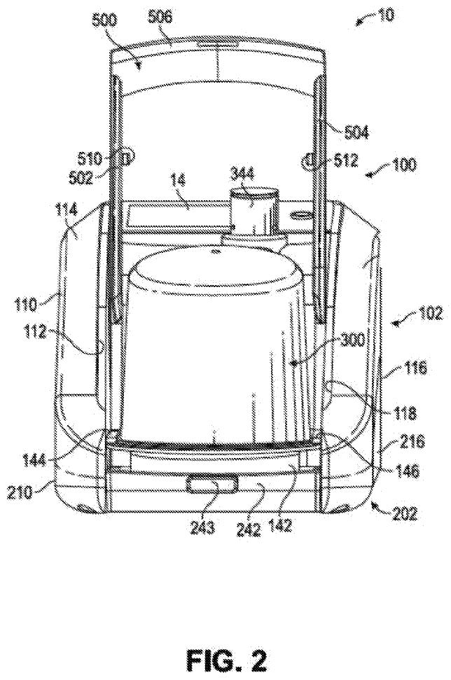

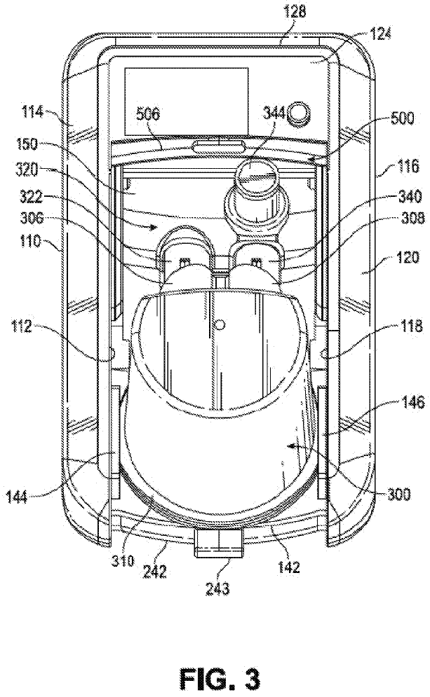

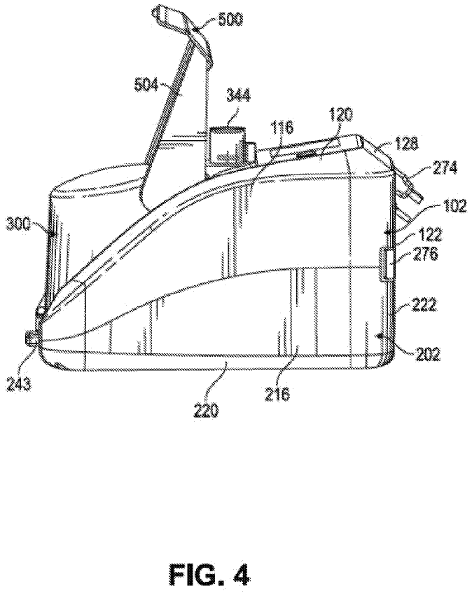

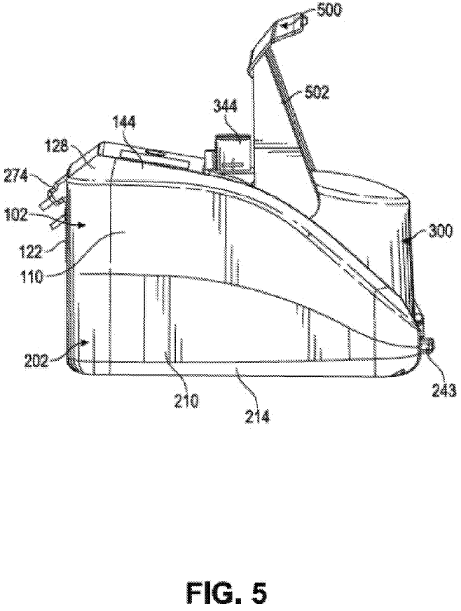

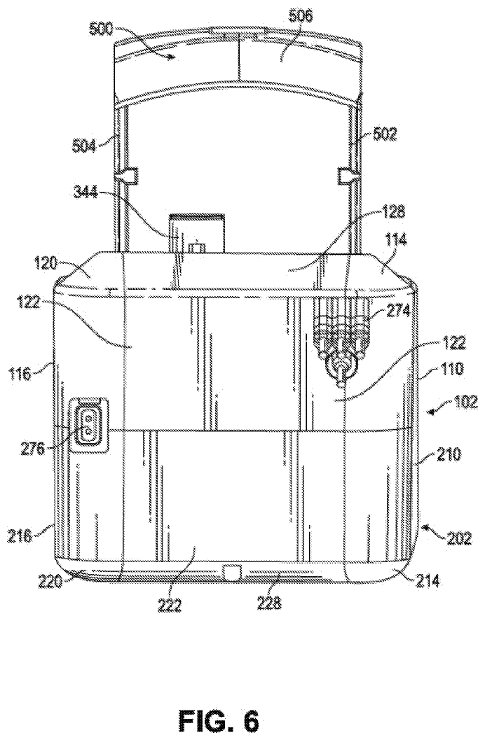

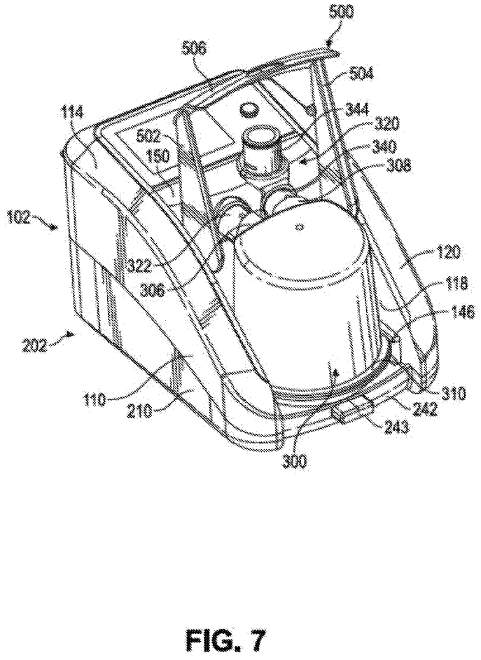

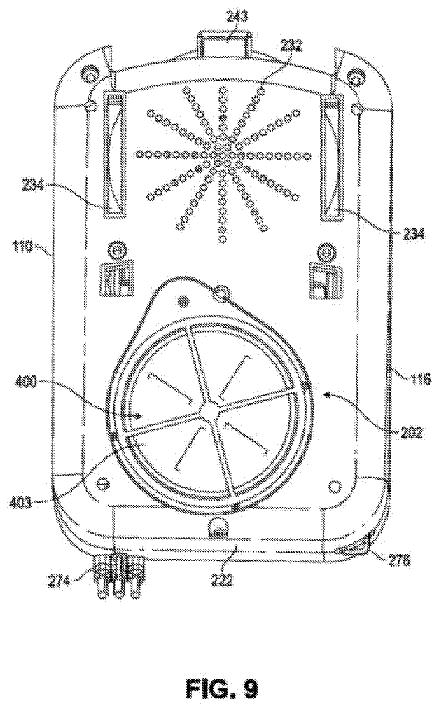

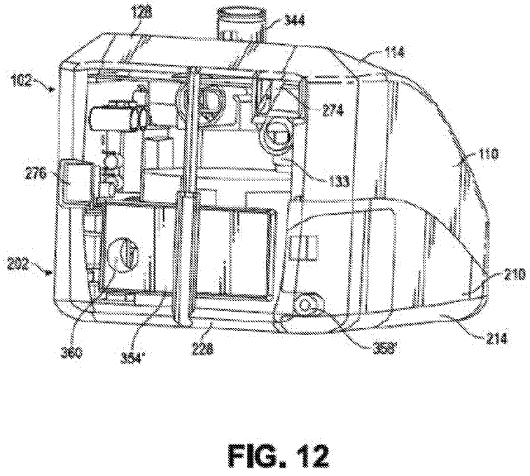

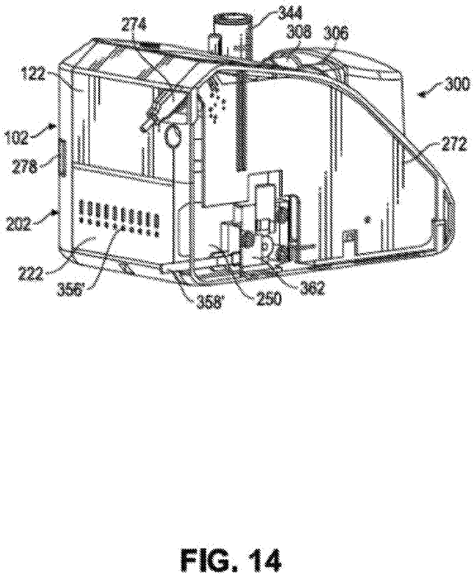

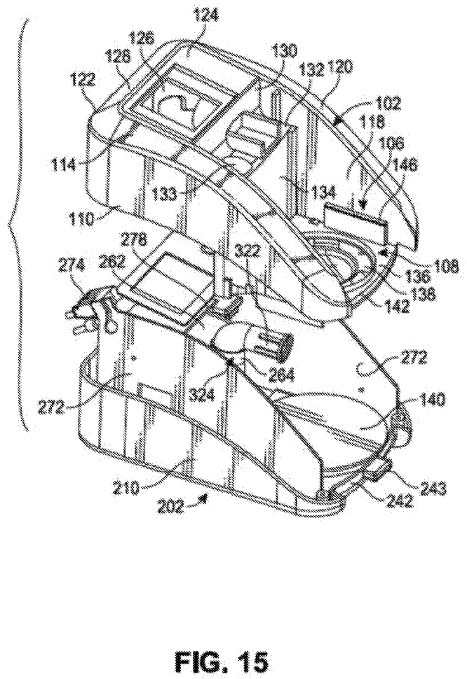

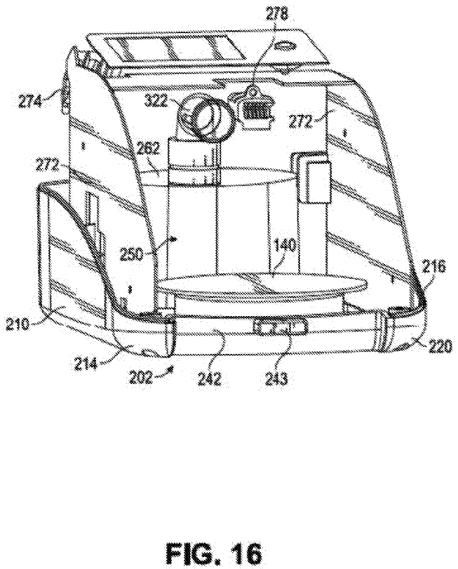

FIGS. 2 to 18 show an example breathing assistance apparatus 10 having a main housing 100. The main housing 100 has a main housing upper chassis 102 and a main housing lower chassis 202. The main housing upper chassis 102 has a peripheral wall arrangement 106 (see FIG. 15). The peripheral wall arrangement defines a humidifier or humidification chamber bay 108 for receipt of a removable humidification chamber 300.

The removable humidification chamber 300 contains a suitable liquid such as water for humidifying gases that can be delivered to a patient.

In the form shown, the peripheral wall arrangement 106 of the main housing upper chassis 102 can include a substantially vertical left side outer wall 110 that is oriented in a front-to-rear direction of the main housing 100, a substantially vertical left side inner wall 112 that is oriented in a front-to-rear direction of the main housing 100, and an interconnecting wall 114 that extends between and interconnects the upper ends of the left side inner and outer walls 110, 112. The main housing upper chassis 102 can further include a substantially vertical right side outer wall 116 that is oriented in a front-to-rear direction of the main housing 100, a substantially vertical right side inner wall 118 that is oriented in a front-to-rear direction of the main housing 100, and an interconnecting wall 120 that extends between and interconnects the upper ends of the right side inner and outer walls 116, 118. The interconnecting walls 114, 120 are angled towards respective outer edges of the main housing 100, but can alternatively be substantially horizontal or inwardly angled.

The main housing upper chassis 102 can further include a substantially vertical rear outer wall 122. An upper part of the main housing upper chassis 102 can include a forwardly angled surface 124. The surface 124 can have a recess 126 for receipt of a display and user interface module 54. The display can be configured to display characteristics of sensed gas(es) in real time. The system can display the patient detection status of the patient interface. If the patient is not detected, the controller may not output or can stop outputting the respiratory rate value(s) and/or other parameters for display. The controller can also optionally output a message for display that no patient is detected at block 2708. An example of the message can be a “--” icon. An interconnecting wall 128 can extend between and interconnect the upper end of the rear outer wall 122 and the rear edge of the surface 124.

A substantially vertical wall portion 130 can extend downwardly from a front end of the surface 124. A substantially horizontal wall portion 132 can extend forwardly from a lower end of the wall portion 130 to form a ledge. A substantially vertical wall portion 134 can extend downwardly from a front end of the wall portion 132 and terminate at a substantially horizontal floor portion 136 of the humidification chamber bay 108. The left side inner wall 112, right side inner wall 118, wall portion 134, and floor portion 136 together can define the humidification chamber bay 108. The floor portion 136 of the humidification chamber bay 108 can have a recess 138 to receive a heater arrangement such as a heater plate 140 or other suitable heating element(s) for heating liquid in the humidification chamber 300 for use during a humidification process.

The main housing lower chassis 202 can be attachable to the upper chassis 102, either by suitable fasteners or integrated attachment features such as clips for example. The main housing lower chassis 202 can include a substantially vertical left side outer wall 210 that is oriented in a front-to-rear direction of the main housing 100 and is contiguous with the left side outer wall 110 of the upper chassis 102, and a substantially vertical right side outer wall 216 that is oriented in a front-to-rear direction of the main housing 100 and is contiguous with the right side outer wall 116 of the upper chassis 102. The main housing lower chassis 202 can further include a substantially vertical rear outer wall 222 that is contiguous with the rear outer wall 122 of the upper chassis 102.

The lower housing chassis 202 can have a lip 242 that is contiguous with the lip 142 of the upper housing chassis 102, and also forms part of the recess for receiving the handle portion 506 of the lever 500. The lower lip 242 can include a forwardly directed protrusion 243 that acts as a retainer for the handle portion 506 of the lever 500. Instead of the lever 500, the system can have a spring-loaded guard to retain the humidification chamber 300 in the humidification chamber bay 108.

An underside of the lower housing chassis 202 can include a bottom wall 230. Respective interconnecting walls 214, 220, 228 can extend between and interconnect the substantially vertical walls 210, 216, 222 and the bottom wall 230. The bottom wall 230 can include a grill 232 comprising a plurality of apertures to enable drainage of liquid in case of leakage from the humidification chamber 300 (e.g. from spills). The bottom wall 230 additionally can include elongated forward-rearward oriented slots 234. The slots 234 can additionally enable drainage of liquid in case of leakage from the humidification chamber 300, without the liquid entering the electronics housing. In the illustrated configuration, the slots 234 can be wide and elongate relative to the apertures of the grill 232 to maximize the drainage of liquid.

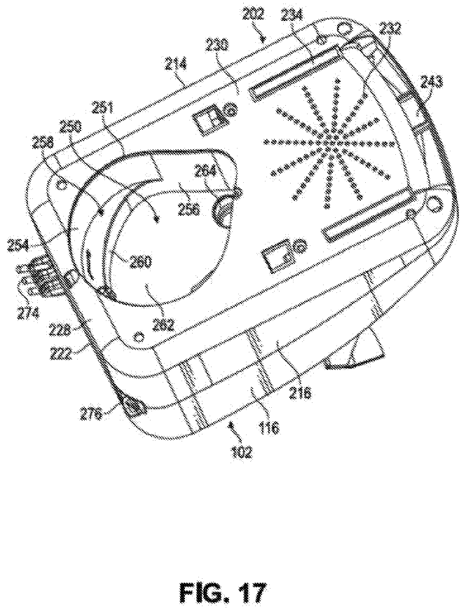

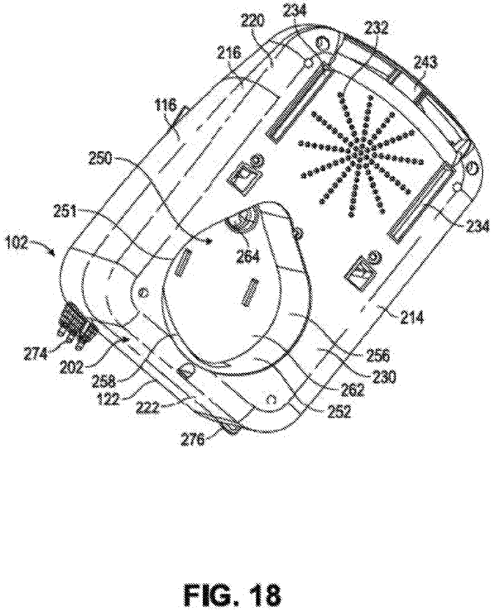

As shown in FIGS. 17 to 18, the lower chassis 202 can have a motor recess 250 for receipt of a motor and sensor module. The motor and sensor module may be non-removable from the main housing 100. The motor and sensor module can be removable from the main housing 100, as illustrated in FIGS. 17-18. A recess opening 251 can be provided in the bottom wall 230 adjacent a rear edge thereof, for receipt of a motor/sensor module. A continuous, gas impermeable, unbroken peripheral wall 252 can be integrally formed with the bottom wall 230 of the lower chassis 202 and extend upwardly from the periphery of the opening 251. A rearward portion 254 of the peripheral wall 252 has a first height, and a forward portion 256 of the peripheral wall 252 has a second height that is greater than the first height. The rearward portion 254 of the peripheral wall 252 terminates at a substantially horizontal step 258, which in turn terminates at an upper auxiliary rearward portion 260 of the peripheral wall 252. The forward portion 256 and upper auxiliary rearward portion 260 of the peripheral wall 252 terminate at a ceiling 262. All of the walls and the ceiling 262 can be continuous, gas impermeable, and unbroken other than the gases flow passage. Therefore, the entire motor recess 250 can be gas impermeable and unbroken, other than the gases flow passage.

The motor and sensor module can be insertable into the recess 250 and attachable to the lower chassis 202. Upon insertion of the motor and sensor module into the lower chassis 202, the gases flow passage tube 264 can extend through the downward extension tube 133 and be sealed by the soft seal.

The humidification chamber 300 can be fluidly coupled to the apparatus 10 in a linear slide-on motion in a rearward direction of the humidification chamber 300 into the chamber bay 108, from a position at the front of the housing 100 in a direction toward the rear of the housing 100. A gases outlet port 322 can be in fluid communication with the motor.

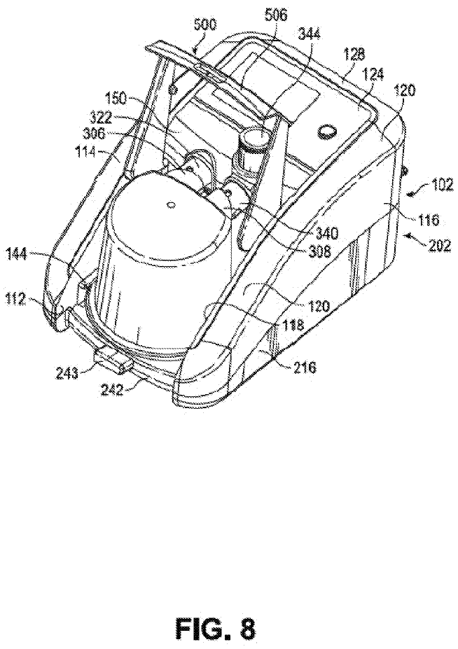

A gases inlet port 340 (humidified gases return) as shown in FIG. 8 can include a removable L-shaped elbow. The removable elbow can further include a patient outlet port 344 for coupling to the patient conduit 16 to deliver gases to the patient interface. The gases outlet port 322, gases inlet port 340, and patient outlet port 344 each can have soft seals such as O-ring seals or T-seals to provide a sealed gases passageway between the apparatus 10, the humidification chamber 300, and the patient conduit 16.

The humidification chamber gases inlet port 306 can be complementary with the gases outlet port 322, and the humidification chamber gases outlet port 308 can be complementary with the gases inlet port 340. The axes of those ports can be parallel to each other to enable the humidification chamber 300 to be inserted into the chamber bay 108 in a linear movement.

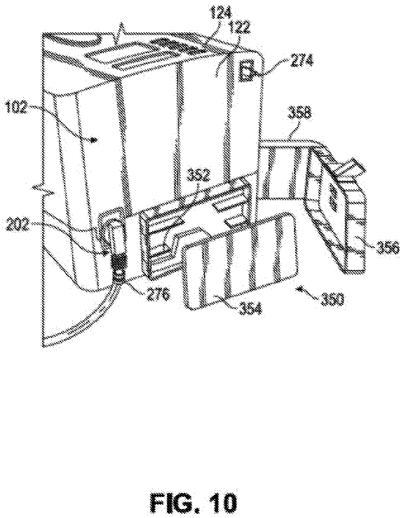

The breathing assistance apparatus can have air and oxygen (or alternative auxiliary gas) inlets in fluid communication with the motor to enable the motor to deliver air, oxygen (or alternative auxiliary gas), or a mixture thereof to the humidification chamber 300 and thereby to the patient. As shown in FIG. 10, the device can have a combined air/oxygen (or alternative auxiliary gas) inlet arrangement 350. This arrangement can include a combined air/oxygen port 352 into the housing 100, a filter 354, and a cover 356 with a hinge 358. A gases tube can also optionally extend laterally or in another appropriate direction and be in fluid communication with an oxygen (or alternative auxiliary gas) source. The port 352 can be fluidly coupled with the motor 402. For example, the port 352 may be coupled with the motor/sensor module 400 via a gases flow passage between the port 352 and an inlet aperture or port in the motor and sensor module 400, which in turn would lead to the motor.

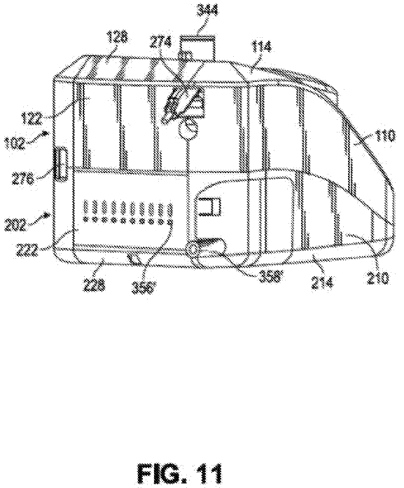

The device can have the arrangement shown in FIGS. 11 to 14 to enable the blower to deliver air, oxygen (or alternative auxiliary gas), or a suitable mixture thereof to the humidification chamber 300 and thereby to the patient. This arrangement can include an air inlet 356′ in the rear wall 222 of the lower chassis 202 of the housing 100. The air inlet 356′ comprises a rigid plate with a suitable grill arrangement of apertures and/or slots. Sound dampening foam may be provided adjacent the plate on the interior side of the plate. An air filter box 354′ can be positioned adjacent the air inlet 356′ internally in the main housing 100, and include an air outlet port 360 to deliver filtered air to the motor via an air inlet port 404 in the motor/sensor module 400. The air filter box 354′ may include a filter configured to remove particulates (e.g. dust) and/or pathogens (e.g. viruses or bacteria) from the gases flow. A soft seal such as an O-ring seal can be provided between the air outlet port 360 and air inlet port 404 to seal between the components. The device can include a separate oxygen inlet port 358′ positioned adjacent one side of the housing 100 at a rear end thereof, the oxygen port 358′ for receipt of oxygen from an oxygen source such as a tank or source of piped oxygen. The oxygen inlet port 358′ is in fluid communication with a valve 362. The valve 362 can suitably be a solenoid valve that enables the control of the amount of oxygen that is added to the gases flow that is delivered to the humidification chamber 300. The oxygen port 358′ and valve 362 may be used with other auxiliary gases to control the addition of other auxiliary gases to the gases flow. The other auxiliary gases can include any one or more of a number of gases useful for gas therapy, including but not limited to heliox and nitric oxide.

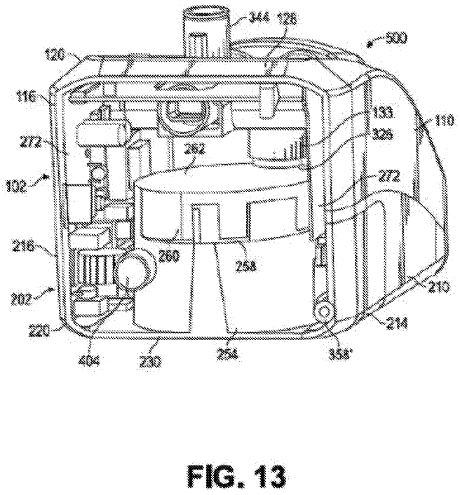

As shown in FIGS. 13 to 16, the lower housing chassis 202 can include one or more suitable electronics boards, such as sensing circuit boards. The electronics boards can be positioned adjacent respective outer side walls 210, 216 of the lower housing chassis 202. The electronics boards can contain, or can be in electrical communication with, suitable electrical or electronics components, such as but not limited to microprocessors, capacitors, resistors, diodes, operational amplifiers, comparators, and switches. Sensors can be used with the electronic boards. Components of the electronics boards (such as but not limited to one or more microprocessors) can act as the controller 19 of the apparatus.

One or more of the electronics boards can be in electrical communication with the electrical components of the apparatus 10, including the display unit and user interface 54, motor, valve 362, and the heater plate 140 to operate the motor to provide the desired flow rate of gases, operate the humidification chamber 300 to humidify and heat the gases flow to an appropriate level, and supply appropriate quantities of oxygen (or quantities of an alternative auxiliary gas) to the gases flow.

The electronics boards can be in electrical communication with a connector arrangement 274 projecting from the rear wall 122 of the upper housing chassis 102. The connector arrangement 274 may be coupled to an alarm, pulse oximetry port, and/or other suitable accessories. The electronics boards can also be in electrical communication with an electrical connector 276 that can also be provided in the rear wall 122 of the upper housing chassis 102 to provide mains or battery power to the components of the device.

As mentioned above, operation sensors, such as flow, temperature, humidity, and/or pressure sensors can be placed in various locations in the breathing assistance apparatus, the patient breathing conduit 16, and/or cannula 51 such as shown in FIG. 1. The electronics boards can be in electrical communication with those sensors. Output from the sensors can be received by the controller 19, to assist the controller 19 to operate the breathing assistance apparatus 10 in a manner that provides optimal therapy, for example including meeting inspiratory demand. In the illustrated embodiment the sensors are positioned on electronic boards that are positioned within the housing. The sensors are encapsulated within the housing.

As outlined above, the electronics boards and other electrical and electronic components can be pneumatically isolated from the gases flow path to improve safety. The sealing also prevents water ingress.

### 2.3 Control System

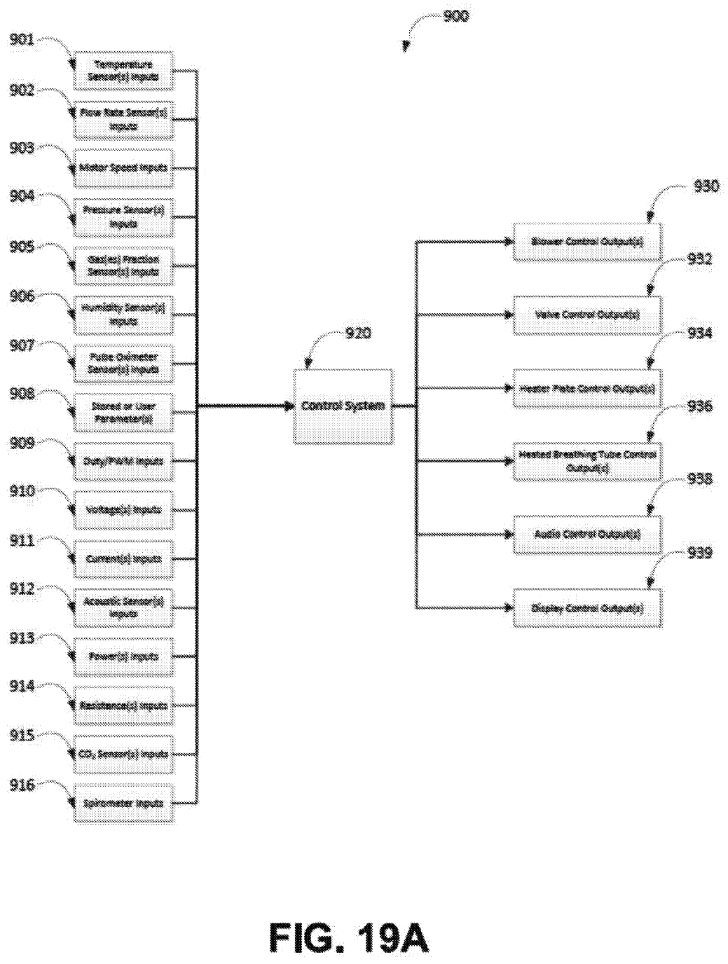

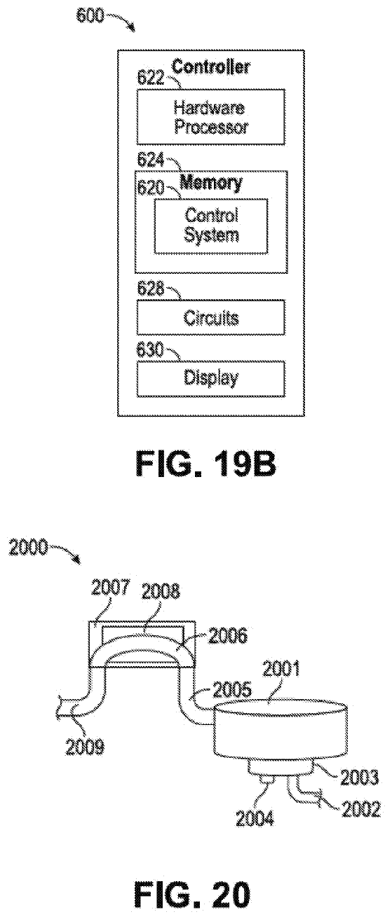

FIG. 19A illustrates a block diagram 900 of an example control system 920 (which can be the controller 19 in FIG. 1) that can detect patient conditions and control operation of the respiratory system including the gases source. The control system 920 can manage a flow rate of the gases flowing through the respiratory system as is the gases are delivered to a patient. For example, the control system 920 can increase or decrease the flow rate by controlling an output of a motor speed of the blower (hereinafter also referred to as a “blower motor”) 930 or an output of a valve 932 in a blender. The control system 920 can automatically determine a set value or a personalized value of the flow rate for a particular patient as discussed below. The flow rate can be optimized by the control system 920 to improve patient comfort and therapy.

The control system 920 can also generate audio and/or display/visual outputs 938, 939. For example, the breathing assistance apparatus can include a display and/or a speaker. The display can indicate to the physicians any warnings or alarms generated by the control system 920. The display can also indicate control parameters that can be adjusted by the physicians. For example, the control system 920 can automatically recommend a flow rate for a particular patient. The control system 920 can also determine a respiratory state of the patient, including but not limited to generating a respiratory rate of the patient, and send it to the display, which will be described in greater detail below.

The control system 920 can change heater control outputs to control one or more of the heating elements (for example, to maintain a temperature set point of the gases delivered to the patient). The control system 920 can also change the operation or duty cycle of the heating elements. The heater control outputs can include heater plate control output(s) 934 and heated breathing tube control output(s) 936.

The control system 920 can determine the outputs 930-939 based on one or more received inputs 901-916. The inputs 901-916 can correspond to sensor measurements received automatically by the controller 600 (shown in FIG. 19B). The control system 920 can receive sensor inputs including but not limited to temperature sensor(s) inputs 901, flow rate sensor(s) inputs 902, motor speed inputs 903, pressure sensor(s) inputs 904, gas(s) fraction sensor(s) inputs 905, humidity sensor(s) inputs 906, pulse oximeter (for example, SpO₂) sensor(s) inputs 907, stored or user parameter(s) 908, duty cycle or pulse width modulation (PWM) inputs 909, voltage(s) inputs 910, current(s) inputs 911, acoustic sensor(s) inputs 912, power(s) inputs 913, resistance(s) inputs 914, CO₂ sensor(s) inputs 915, and/or spirometer inputs 916. The control system 920 can receive inputs from the user or stored parameter values in a memory 624 (shown in FIG. 19B). The control system 920 can dynamically adjust flow rate for a patient over the time of their therapy. The control system 920 can continuously detect system parameters and patient parameters. A person of ordinary skill in the art will appreciate based on the disclosure herein that any other suitable inputs and/or outputs can be used with the control system 920.

### Pressure Sensor or Sensors

In one configuration, the apparatus can have one or more pressure sensors. One or more pressure sensors may be provided to sense or measure a pressure characteristic of the flow of gases in the flow path of the apparatus and generated respective pressure variables, such as pressure sensor signals or data. The pressure sensors may include any type of suitable pressure sensor including, but not limited to, a gauge pressure sensor and/or an absolute pressure sensor.

A gauge pressure sensor may be configured to sense the gauge pressure of the flow of gases and generates a representative gauge pressure variable, such as a gauge pressure signal or pressure data. The gauge pressure may represent the pressure of the flow of gases in the flow path with reference or relative to atmospheric pressure. For example, the gauge pressure may represent the difference between the absolute pressure inside the flow path and the absolute pressure inside the housing (i.e. atmospheric or ambient pressure).

An absolute pressure sensor may be configured to sense the absolute pressure of the flow of gases and generates a representative absolute pressure variable, such as an absolute pressure signal or pressure data. The absolute pressure may represent the pressure of the flow of gases in the flow path with reference or relative to a vacuum.

As will be appreciated by a skilled person, the one or more pressure sensors that are configured to sense or measure a pressure characteristic of the flow of gases may be directly in or at least partially immersed in the main or bulk flow path of the flow of gases (e.g. the sensors may be part of or exposed to a sensor passage or sensor chamber that forms part of the main or bulk flow path), or directly in or at least partially immersed in a secondary or sample flow path that is operatively or fluidly connected to the main or bulk flow path, or otherwise operatively or fluidly couple or connected to the flow of gases in the flow path.

The pressure sensors for sensing pressure characteristics of the flow of gases may be independently mounted within the housing of the apparatus and electrically connected or otherwise in data communication with the controller or control system, or may be mounted or coupled to a sensor circuit board or other circuit board associated with the flow path of the flow of gases. In one configuration, the pressure sensors may be located or configured to sense the pressure of the flow of gases at a location along the flow path before (e.g. upstream of) the humidifier or humidification chamber. In another configuration, the pressure sensor(s) may be located or configured to sense the pressure of the flow of gases at a location along the flow path between the flow generator, e.g. blower, and humidifier chamber, for example at a location along the flow path between the blower outlet and humidifier chamber inlet (i.e. downstream of the blower and upstream of the humidifier chamber).

One or more pressures may also be provided for sensing other pressures associated with the apparatus, such as the ambient environment in which it is located. In one configuration, the apparatus may be provided with an ambient pressure sensor that is configured to sense or measure the ambient or atmospheric pressure of the local ambient environment in which the apparatus is located and generates a representative ambient pressure variable, such as an ambient pressure signal or pressure data. In one configuration, the ambient pressure sensor may be an absolute pressure located or positioned on or in the housing and which is configured to sense the ambient or atmospheric pressure of the environment the apparatus is located in.

In one configuration, the apparatus may be provided with a gauge pressure signal that generates a gauge pressure signal or data representing the gauge pressure associated with the flow of gases in the flow path.

In another configuration, the apparatus may be provided with a gauge pressure sensor configured to generate a gauge pressure signal or data representing the gauge pressure associated with the flow of gases, and an ambient pressure sensor configured to generate an ambient pressure signal or data. In such a configuration, the apparatus can be configured to utilise the ambient pressure data as an input to a correction algorithm or factor or function applied to the sensed gauge pressure signal or data. For example, the correction algorithm, factor or function may be configured to correct the sensed gauge pressure signal or data to take into account the impact of changing air density on the sensed gauge pressure signal or data.

### 2.4 Controller

FIG. 19B illustrates a block diagram of an embodiment of a controller 600 (which can be the controller 19 in FIG. 1). The controller 600 can include programming instructions for detection of input conditions and control of output conditions. The programming instructions can be stored in the memory 624 of the controller 600. The programming instructions can correspond to the methods, processes and functions described herein. The programming instructions can be executed by one or more hardware processors 622 of the controller 600. The programming instructions can be implemented in C, C++, JAVA, or any other suitable programming languages. Some or all of the portions of the programming instructions can be implemented in application specific circuitry 628 such as ASICs and FPGAs.

The controller 600 can also include circuits 628 for receiving sensor signals. The controller 600 can further include a display 630 for transmitting status of the patient and the respiratory assistance system. The display 630 can also show warnings and/or other alerts. The display 630 can be configured to display characteristics of sensed gas(es) in real time or otherwise. The controller 600 can also receive user inputs via the user interface such as display 630. The user interface can include button(s) and/or dial(s). The user interface can comprise a touch screen.

### 2.5 Motor and Sensor Module

Any of the features of the respiratory system described herein, including but not limited to the humidification chamber, the flow generator, the user interface, the controller, and the patient breathing conduit configured to couple the gases flow outlet of the respiratory system to the patient interface, can be combined with any of the sensor modules described herein.

FIG. 20 illustrates a block diagram of the motor and sensor module 2000, which can be received by the recess 250 in the breathing assistance apparatus (shown in FIGS. 17 and 18). The motor and sensor module can include a blower 2001, which entrains room air to deliver to a patient. The blower 2001 can be a centrifugal blower.

One or more sensors (for example, Hall-effect sensors) may be used to measure a motor speed of the blower motor. The blower motor may comprise a brushless DC motor, from which motor speed can be measured without the use of separate sensors. For example, during operation of a brushless DC motor, back-EMF can be measured from the non-energized windings of the motor, from which a motor position can be determined, which can in turn be used to calculate a motor speed. In addition, a motor driver may be used to measure motor current, which can be used with the measured motor speed to calculate a motor torque. The blower motor may comprise a low inertia motor.

Room air can enter a room air inlet 2002, which enters the blower 2001 through an inlet port 2003. The inlet port 2003 can include a valve 2004 through which a pressurized gas may enter the blower 2001. The valve 2004 can control a flow of oxygen into the blower 2001. The valve 2004 can be any type of valve, including a proportional valve or a binary valve. In some embodiments, the inlet port does not include a valve.

The blower 2001 can operate at a motor speed of greater than 1,000 RPM and less than 30,000 RPM, greater than 2,000 RPM and less than 21,000 RPM, or between any of the foregoing values. Operation of the blower 2001 mixes the gases entering the blower 2001 through the inlet port 2003. Using the blower 2001 as the mixer can decrease the pressure drop that would otherwise occur in a system with a separate mixer, such as a static mixer comprising baffles, because mixing requires energy.

The mixed air can exit the blower 2001 through a conduit 2005 and enters the flow path 2006 in the sensor chamber 2007. A sensing circuit board with sensors 2008 can positioned in the sensor chamber 2007 such that the sensing circuit board is at least partially immersed in the gases flow. At least some of the sensors 2008 on the sensing circuit board can be positioned within the gases flow to measure gases properties within the flow. After passing through the flow path 2006 in the sensor chamber 2007, the gases can exit 2009 to the humidification chamber.

Positioning sensors 2008 downstream of the combined blower and mixer 2001 can increase accuracy of measurements, such as the measurement of gases fraction concentration, including oxygen concentration, over systems that position the sensors upstream of the blower and/or the mixer. Such a positioning can give a repeatable flow profile. Further, positioning the sensors downstream of the combined blower and mixer avoids the pressure drop that would otherwise occur, as where sensing occurs prior to the blower, a separate mixer, such as a static mixer with baffles, is required between the inlet and the sensing system. The mixer can introduce a pressure drop across the mixer.

Positioning the sensing after the blower can allow the blower to be a mixer, and while a static mixer would lower pressure, in contrast, a blower increases pressure. Also, immersing at least part of the sensing circuit board and sensors 2008 in the flow path can increase the accuracy of measurements because the sensors being immersed in the flow means they are more likely to be subject to the same conditions, such as temperature and pressure, as the gases flow and therefore provide a better representation of the gases flow characteristics.

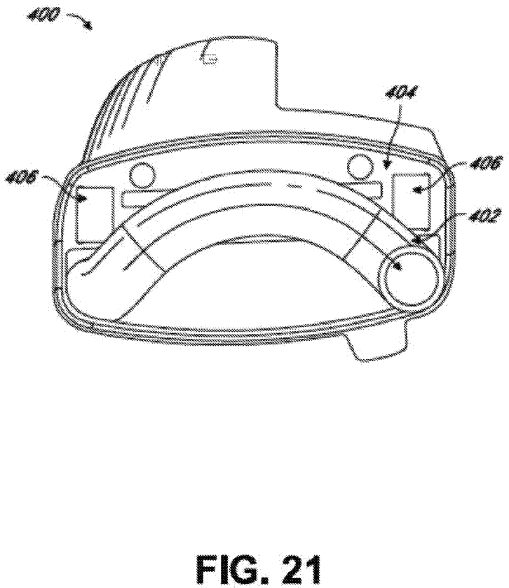

Referring to FIG. 21, the gases exiting the blower can enter a flow path 402 in a sensor chamber 400, which can be positioned within the motor and sensor module and can be the sensor chamber 2007 of FIG. 20. The flow path 402 can have a curved shape. The flow path 402 can be configured to have a curved shape with no sharp turns. The flow path 402 can have curved ends with a straighter section between the curved ends. A curved flow path shape can reduce pressure drop in a gases flow without reducing the sensitivity of flow measurements by partially coinciding a measuring region with the flow path to form a measurement portion of the flow path.

A sensing circuit board 404 with sensors, such as acoustic transmitters and/or receivers, humidity sensor, temperature sensor, pressure sensor(s), thermistor, and the like, can be positioned in the sensor chamber 400 such that the sensing circuit board 404 is at least partially immersed in the flow path 402. Immersing at least part of the sensing circuit board and sensors in the flow path can increase the accuracy of measurements because the sensors immersed in the flow are more likely to be subject to the same conditions, such as temperature and pressure, as the gases flow, and therefore provide a better representation of the characteristics of the gases flow. After passing through the flow path 402 in the sensor chamber 400, the gases can exit to the humidification chamber. Alternatively, one or more of the pressure sensor(s) may be provided on one or more separate circuit boards that are positioned or located so as to enable the pressure sensor(s) to measure or sense pressure characteristics associated with the flow of gases and/or ambient pressure.

The gases flow rate may be measured using at least two different types of sensors. The first type of sensor can comprise a thermistor, which can determine a flow rate by monitoring heat transfer between the gases flow and the thermistor. The thermistor flow sensor can run the thermistor at a constant target temperature within the flow when the gases flow around and past the thermistor. The sensor can measure an amount of power required to maintain the thermistor at the target temperature. The target temperature can be configured to be higher than a temperature of the gases flow, such that more power is required to maintain the thermistor at the target temperature at a higher flow rate.

The thermistor flow rate sensor can also maintain a plurality of (for example, two, three, or more) constant temperatures on a thermistor to avoid the difference between the target temperature and the gases flow temperature from being too small or too large. The plurality of different target temperatures can allow the thermistor flow rate sensor to be accurate across a large temperature range of the gases. For example, the thermistor circuit can be configured to be able to switch between two different target temperatures, such that the temperature of the gases flow will always fall within a certain range relative to one of the two target temperatures (for example, not too close but not too far). The thermistor circuit can be configured to operate at a first target temperature of about 50° C. to about 70° C., or about 66° C. The first target temperature can be associated with a desirable flow temperature range of between about 0° C. to about 60° C., or about 0° C. and about 40° C. The thermistor circuit can be configured to operate at a second target temperature of about 90° C. to about 110° C., or about 100° C. The second target temperature can be associated with a desirable flow temperature range of between about 20° C. to about 100° C., or about 30° C. and about 70° C.

The controller can be configured to adjust the thermistor circuit to change between at least the first and second target temperature modes by connecting or bypassing a resistor within the thermistor circuit. The thermistor circuit can be arranged as a Wheatstone bridge configuration comprising a first voltage divider arm and a second voltage divider arm. The thermistor can be located on one of the voltage divider arms. More details of a thermistor flow rate sensor are described in PCT Application Publication No. WO2018/052320, filed 3 Sep. 2017, which is incorporated by reference herein in its entirety.

The second type of sensor can comprise an acoustic sensor assembly. Acoustic sensors including acoustic transmitters and/or receivers can be used to measure a time of flight of acoustic signals to determine gases velocity and/or composition, which can be used in breathing assistance apparatuses. In one ultrasonic sensing (including ultrasonic transmitters and/or receivers) topology, a driver causes a first sensor, such as an ultrasonic transducer, to produce an ultrasonic pulse in a first direction. A second sensor, such as a second ultrasonic transducer, receives this pulse and provides a measurement of the time of flight of the pulse between the first and second ultrasonic transducers. Using this time of flight measurement, the speed of sound of the gases flow between the ultrasonic transducers can be calculated by a processor or controller of the respiratory system. The second sensor can transmit and the first sensor can receive a pulse in a second direction opposite the first direction to provide a second measurement of the time of flight, allowing characteristics of the gases flow, such as a flow rate or velocity, to be determined. In another acoustic sensing topology, acoustic pulses transmitted by an acoustic transmitter, such as an ultrasonic transducer, can be received by acoustic receivers, such as microphones. More details of an acoustic flow rate sensor are described in PCT Application Publication WO2017/095241, filed 2 Dec. 2016, which is incorporated by reference herein in its entirety.

Readings from both the first and second types of sensors can be combined to determine a more accurate flow measurement. For example, a previously determined flow rate and one or more outputs from one of the types of sensor can be used to determine a predicted current flow rate. The predicted current flow rate can then be updated using one or more outputs from the other one of the first and second types of sensor, in order to calculate a final flow rate.

### 3. Example Measurement Device Embodiments

Example embodiments of the measurement device for use in undertaking lung performance or lung function measurements and/or diagnosis will be described in the context of the example breathing assistance apparatus 10 described above, which is configured or operable as a flow therapy apparatus to provide nasal high flow therapy via an unsealed patient interface. However, as explained earlier, the measurement device may also be used similarly with any form or type of breathing assistance apparatus with a controllable flow generator and one or more sensors for sensing characteristics of the flow of gases.

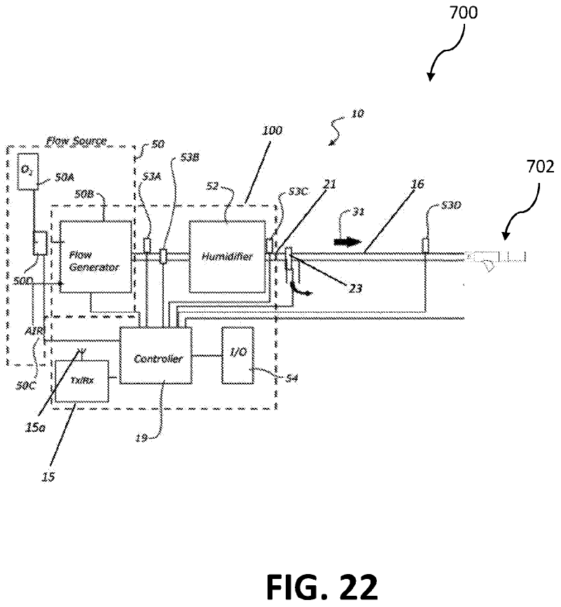

Referring to FIG. 22, an example embodiment of a measurement system 700 for conducting lung performance or lung function measurements and/or patient diagnosis and/or patient assessments is provided. The measurement system 700 is provided using a measurement device 702 in combination with the breathing assistance apparatus 10. As shown, in this example, the measurement device 702 is a component or attachment that can be releasably or removably connected or attached to an end of the patient breathing conduit 16. Once the measurement device is connected or attached to the breathing conduit 16, the breathing assistance apparatus may be operated in an apparatus or patient diagnosis mode so that one or lung function measurements or assessments can be performed with the patient. Example embodiments of the configuration of the measurement device, apparatus or patient diagnostic mode, and process of using the measurement device with the breathing assistance apparatus will be described in further detail below.

In one example configuration, the measurement device is configured to attach or connect or otherwise fluidly couple to the flow path or flow of gases generated by a breathing assistance apparatus comprising a flow generator. The breathing assistance apparatus comprises one or more sensors that measure or sense characteristics of the flow of gases. The breathing assistance apparatus can be operated in a mode that delivers a flow of gases at a controlled flow rate to the measurement device to provide a desired pneumatic resistance. Lung performance measurements may then be performed based on recording and processing the sensor data from the one or more sensors of the apparatus as the user performs one or more exhalation manoeuvres into a mouthpiece of the measurement device. In this configuration, the breathing assistance apparatus can be used or operated as a diagnosis or measurement system when used with the measurement device attachment. In this example configuration, the measurement device need not necessarily have any sensors or electronics, as the measurement data is obtained from the one or more sensors of the breathing assistance apparatus and/or breathing conduit. This configuration enables the measurement device attachment to leverage off the existing sensors and sensing capability of the breathing assistance apparatus, as will be explained further below. This configuration allows for a lower-cost measurement device attachment to be manufactured that is primarily a mechanical component and which can be used with the breathing assistance apparatus to perform lung performance measurements using the pre-existing one or more sensors of the breathing assistance apparatus., i.e. the measurement device does not need onboard sensors or sensing capability. Additionally, this configuration enables the measurement device to leverage off the pneumatic resistance provided by the flow of gases generated by the flow generator.

In the embodiment described below, the measurement system 700 employs the measurement device 702 attaching to the flow path of the breathing assistance apparatus 10, and leverages off the existing flow path sensors of the breathing assistance apparatus to measure or sense one or more characteristics of the flow of gases during a diagnosis mode as the user performs forced expiratory lung manoeuvres into the measurement device. The sensors may, for example, include sensors for sensing any one or more of the following gas flow characteristics: flow rate, pressure, temperature, humidity, gas concentration or any other characteristic that is useful in lung performance assessments. The sensor data obtained from the sensors during the diagnosis mode can be processed and/or analysed to generate one or more lung performance measurements, metrics, and/or assessments. In this embodiment, the sensors are external to the measurement device 702, and are typically located in the main housing 100 of the breathing assistance apparatus and/or along the patient breathing conduit 16.

### 3.1. First Example Embodiment—Mechanical Configuration of Measurement Device

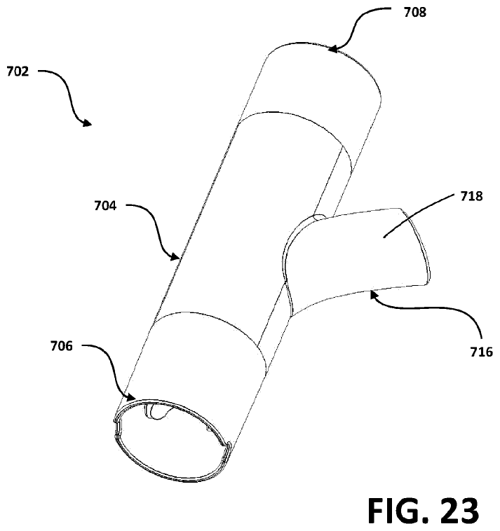

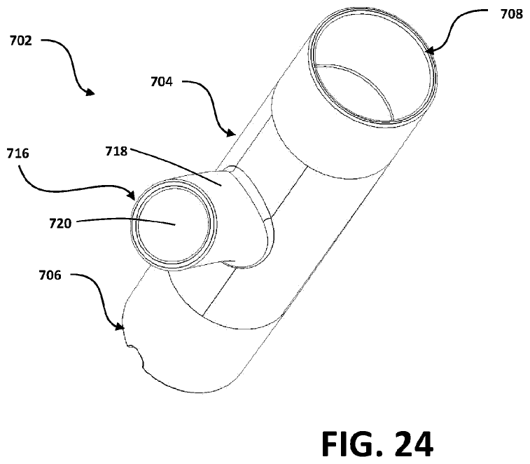

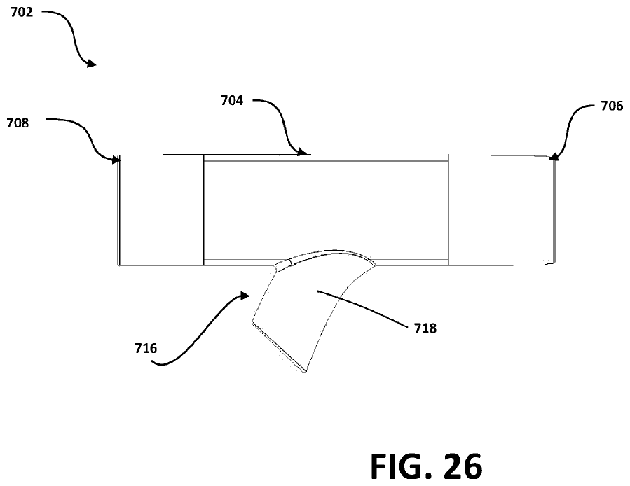

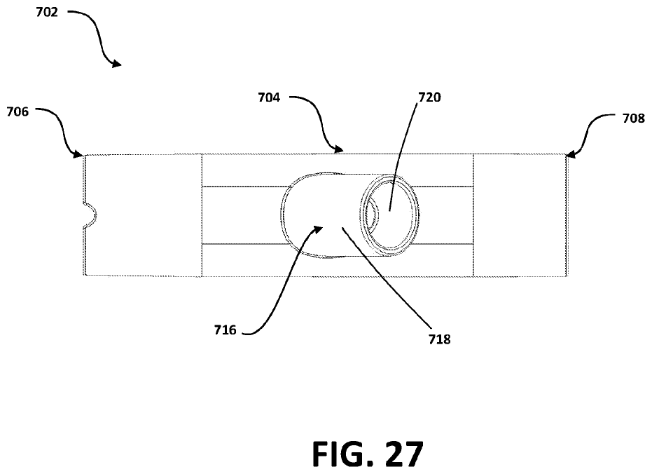

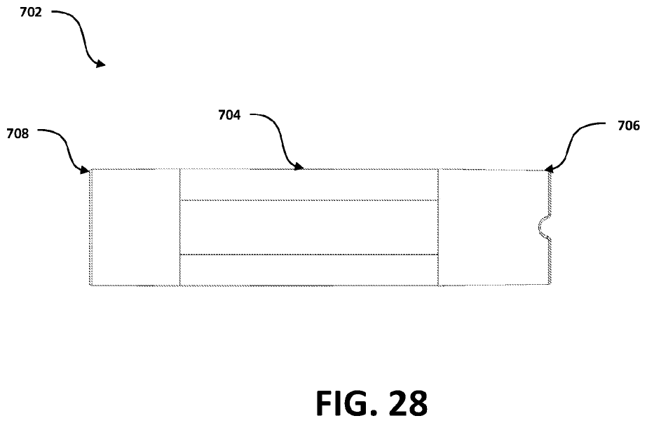

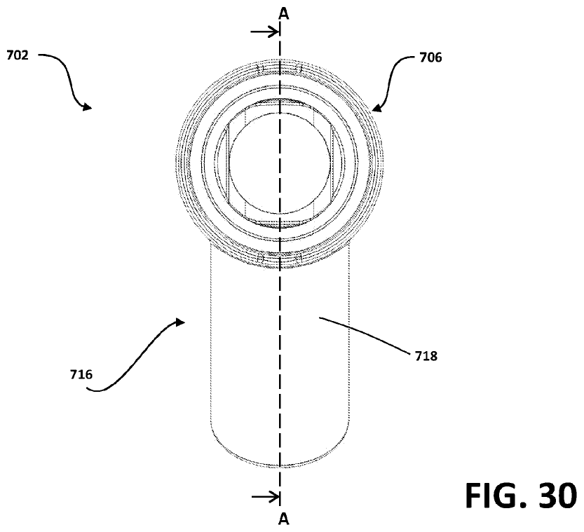

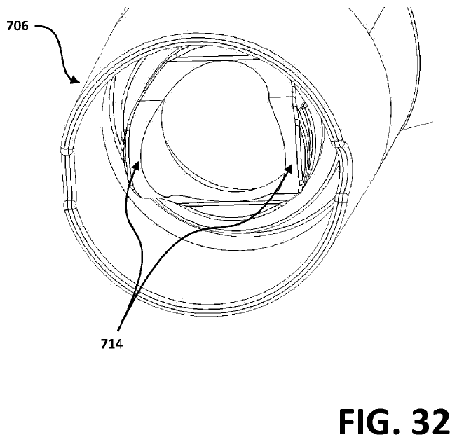

Referring to FIGS. 23-32, a first example embodiment of the measurement device 702 will be described in further detail. In this embodiment, the measurement device 702 comprises a main body 704 that extends between a first end 706 and a second end 708.

The first end 706 of the main body is a connector end, and the second end 708 is a mouthpiece end.

In this embodiment, the main body 704 is a component in the form of a conduit, tube or tubular component, or manifold component. The main body has a main lumen or lumens extending between its connector end 706 and mouthpiece end 708 to allow a flow of gases to flow or be conveyed along the main body 704 between its ends. The main lumen or lumens are in the form of a passage or channel or internal space extending between the openings at the connector end 706 and mouthpiece end 708.

### Main Body and Main Lumen

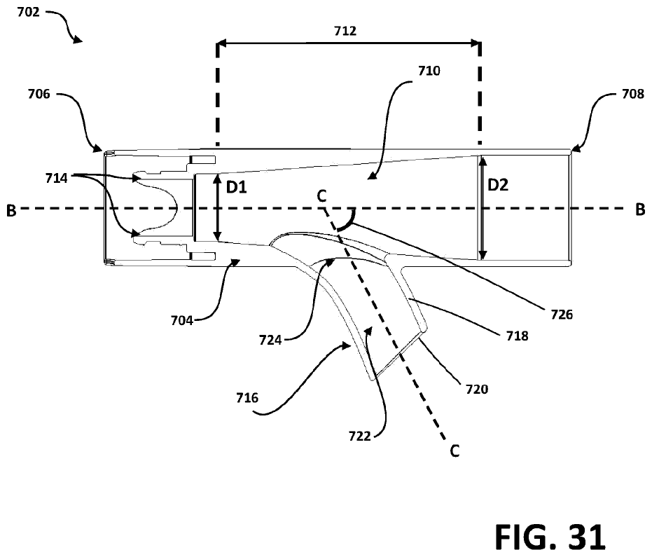

Referring to FIG. 31, in this embodiment, the main body 704 comprises a single main lumen generally indicated at 710. The main lumen 710 is a passage or channel extending along the length of the main body 704 between the open ends 706, 708 of the main body. The shape and/or dimensions of the main lumen 710 may be uniform or non-uniform along the length of the main body 704. For example, the inner dimensions or diameter of the main lumen 710 may be uniform or vary along the length of the main body 704. The main lumen 710 is generally defined by the surrounding peripheral wall of the main body and/or any internal features within the main body.

In this embodiment, the internal diameter of at least a portion of the main lumen 710 varies along the length of the main body 704. Referring to FIG. 31, a central portion of the main lumen generally indicated at 712 widens in diameter from the connector end 706 to the mouthpiece end 708. For example, the diameter shown at D1 at or toward the connector end is smaller than the diameter shown at D2 at or toward the mouthpiece end. As shown, the diameter progressively or gradually widens from D1 to D2, in this embodiment. In one example, the dimension or diameter D1 may be approximately 13 mm and the dimension or diameter D2 may be approximately 20 mm, but it will be appreciated alternative dimensions or diameters may be used, depending on the characteristics of the breathing conduit for connection and/or other design factors. This configuration can also be described as a portion of the main lumen 712 tapering or narrowing in diameter or cross-sectional area from the mouthpiece end 708 toward the connector end 706. In other embodiments, it will be appreciated that the internal profile, cross-sectional area or diameter of the main lumen 710 may vary in alternative arrangements or configurations, including widening, narrowing, or a combination of these along one or more portions or the entire length of the main body 704. The variations or transitions in diameter or profile of the main lumen 710 may be gradual or progressive, or sharper or with stepped changes.

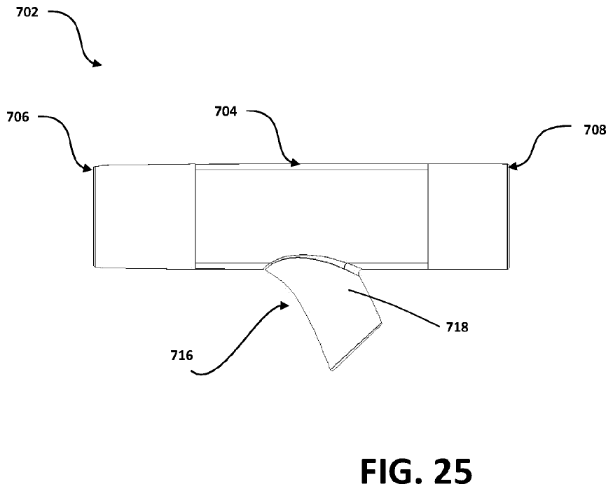

In this embodiment, the main body 704 is an elongate component. The main body 704 is substantially hollow and defined by a conduit or peripheral wall that extends between the ends 706, 708 of the main body. In this embodiment, the main body has a substantially circular cross-section along its length as shown in FIGS. 23, 24, 29 and 30 for example. In one configuration, the outer dimensions or diameter of the main body 704 may be substantially uniform along its length, as shown in FIGS. 25-28. In alternative configurations, the outer dimensions or diameter of the main body may vary along its length.

In this embodiment, the main body has a substantially cylindrical form-factor or shape, with an outer surface defined by circular cross-sectional profile or shape along its length. It will be appreciated that the main body may be provided in alternative shapes or configurations in other embodiments. For example, the cross-sectional profile of the outer surface of the main body may be circular, oval, rectangular, square, arbitrary or any suitable shape or combination of shapes along the length of the main body.

In this embodiment, the main body 704 and main lumen can generally be defined or aligned along or with reference to a central longitudinal axis. The main body and main lumen are straight and extend in a single axis or dimension. In alternative embodiments, it will be appreciated that the main body and/or main lumen may have alternative shapes and configurations such as, but not limited to, curvilinear, arcuate, elbow-configuration, or may otherwise have a non-straight profile that does not conform or align to a single longitudinal axis or dimension. It will be appreciated that various shapes and configurations of the main body can be provided with a main lumen that fluidly communicates between the connector end and mouthpiece end of the main body.

### Connector End

Referring to FIGS. 22 and 30-32, the connector end 706 of the measurement device 702 will be described in more detail.

In this embodiment, the connector end 706 is configured to releasably connect or attach to the end of a breathing conduit 16 or tube of the breathing assistance apparatus 10. As will be appreciated, the breathing conduit 16 of a breathing assistance apparatus is a typically a flexible conduit that attaches or connects at one end to a gases outlet 21 of the breathing assistance apparatus such that it is fluidly connected or in fluid communication with the flow of gases generated by the flow generator 11 of the apparatus. The other end of the breathing conduit 16 typically provides a connector for releasably connecting or coupling to a patient interface (e.g. nasal canular, nasal mask, full face mask, tracheostomy interface or similar) to deliver the flow of gases to a patient's airway when the breathing assistance apparatus is used for respiratory therapy (e.g. high flow therapy, PAP therapy or similar), e.g. in normal therapy mode. In this embodiment, the connector end 706 of the measurement device 702 is configured or arranged to releasably connect or attach to the connector or end of the breathing conduit such that the measurement device is in fluid communication with the flow of gases conveyed along the breathing conduit.

In this embodiment, the connector end 706 of the measurement device 702 comprises a connecting structure or arrangement generally indicated at 714 that is configured to provide a releasable fluid connection to the end or connector of a breathing conduit 16. The end or connector of the breathing conduit may comprise a complementary connecting structure or arrangement for engaging and attaching with the connector end 706 of the measurement device 702 for coupling the two components together in a releasable manner. In this embodiment, the connecting structure 714 may be provided by a pair of opposing resilient clipping protrusions that releasably engage or clip into corresponding formations or indents or catches or complementary end or connector of the breathing conduit 16. It will be appreciated that the connector end 706 may be configured, arranged or provided with any suitable form of mechanical releasably fastening or coupling to complement the end or a connector of the breathing conduit including, but not limited to, screw threaded, rotational locking or coupling, clip fittings, snap-fit connection, push-fit connection, interference-fit connection, latch connection, or the like.

In some embodiments, the connector end 706 may be provided with a connecting structure that is compatible with connecting to the end of one or more specific types of breathing conduits, including brand or manufacturer-specific breathing conduits. For example, in one embodiment, the connector end 706 may be configured to attach or connect to a 20 mm breathing tube used with a high-flow breathing assistance apparatus. In other embodiments, the connector end 706 may be provided with a generic or universal connecting structure or configuration that is operable or able to couple to the end of various or a broad range of different types of breathing conduits.

In the embodiment shown, the connector end 706 is configured for releasable connection or coupling with the end or a connector of the breathing conduit 16. This allows the measurement device to be connected to the breathing conduit to perform patient diagnosis measurements in patient diagnosis mode of the apparatus, and then removed following the measurements, leaving the breathing conduit able to be connected or re-connected to a patient interface for normal respiratory therapy mode.

In alternative embodiments, the connector end 706 of the measurement device may be configured with a non-releasable permanent connection to the breathing tube 16, such that the components can't be released from each other without breakage. Alternatively, a semi-permanent connection may be provided between the connector end 706 of the measurement device and the breathing conduit 16, such that tools or similar are required to release the components from each other.

In further alternative embodiments, the measurement device 702 may be provided with an integral breathing conduit extending from the connector end 706. In such an embodiment, the measurement device 702 is combined with an integral flexible conduit extending from the connector end. The integral flexible conduit may terminate in a connector end which can releasably connect or attach to the gases outlet 21 of the breathing assistance apparatus.

In another embodiment, the measurement device 702 may be configured to releasably connect or attach directly to the gases outlet 16 of the breathing assistance apparatus without an intermediate breathing conduit. In another embodiment, the measurement device 702 may be configured to releasably connect or attach directly to the flow generator outlet or gases outlet port 322, if the humidifier chamber is removed from the apparatus. In another embodiment, the humidifier or humidifier chamber may be bypassed via a bypass conduit or other bypass configuration. For example, the measurement device may attach directly to or indirectly via the breathing conduit to a bypass conduit or port or outlet that bypasses the humidifier or humidifier chamber, such that the measurement device is in fluid communication with the flow of gases from the flow generator outlet, and the humidifier is temporarily cut-out or bypassed from the flow path.

### Mouthpiece End

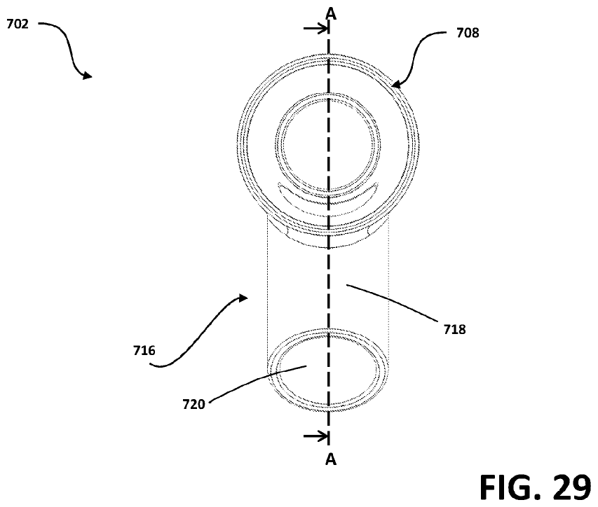

Referring to FIGS. 29 and 31, the mouthpiece end 708 of the measurement device 702 will be described in more detail.

In this embodiment, the mouthpiece end 708 is a portion that is configured to releasably receive and retain an optional removable mouthpiece, to be explained in further detail later. In this embodiment, the mouthpiece end 708 is an open cylindrical portion at the end of the main body 704. The mouthpiece end 708 is non-threaded in this embodiment, but could be threaded in other embodiments. However, it will be appreciated that the mouthpiece end may be any other suitable shape or configuration for receiving and retaining a releasably mouthpiece.

In alternative embodiments, the measurement device 702 may be used directly without a separate removable mouthpiece. In such embodiments, the mouthpiece may be integrally formed with the mouthpiece end 708 or the mouthpiece end 708 itself may be configured as a mouthpiece or used as a mouthpiece for fluidly communicating with the airway of a user or patient when they seal their mouth around the mouthpiece, in use. It will be appreciated that the shape and configuration of the mouthpiece portion or end of the main body may be cylindrical, oval, elliptical, mouth-shaped, or any other suitable shape suitable for a user's mouth.

### Exhaust Vent

In this embodiment, the measurement device 702 comprises one or more exhaust openings that are in fluid communication with the main lumen. The exhaust opening or openings provide a pathway for the flow of gases and/or exhaled breath from the user or patient to escape to atmosphere or the surrounding environment, during use of the measurement device. Without one or more exhaust openings, complete blockage of the flow path may occur in some system configurations when the user places their mouth on the mouthpiece or otherwise creates a seal around the mouthpiece during use. In alternative embodiments, the measurement device may not have any exhaust vents or exhaust openings, and the system configuration may allow for this. For example, exhaust vents, exhaust openings, and/or pressure relief valves may be provided in the flow path upstream of the measurement device.

Referring to FIGS. 23-27 and 29-31, an example of a configuration of an exhaust opening will be described in further detail for this embodiment. In the embodiment shown, the measurement device 702 is provided with an exhaust opening 716. The exhaust opening 716 is located between the connector end 706 and mouthpiece end 708. In this embodiment, the exhaust opening 716 extends from or branches off the main body 704 of the measurement device. In this embodiment, the exhaust opening is in the form of or comprises a vent. In this embodiment, the exhaust opening 716 comprises a single vent. In this embodiment, the exhaust opening is a protruding vent 718 that extends or protrudes from the wall of the main body 704 and terminates in an opening 720 or openings. The protruding vent 718 may be integrally formed with the remainder of the main body or may be formed as a separate component that is then coupled, moulded, or otherwise connected into the main body 704.

Referring to FIG. 31, the protruding vent 718 comprises a lumen generally indicated at 722. The exhaust lumen 722 is fluidly connected or in fluid communication with the main lumen 710 of the main body 704. As shown, the auxiliary or exhaust lumen 722 branches off the main lumen 710. In this embodiment, the exhaust lumen is a passage or channel formed by the wall of the protruding vent 718. The protruding vent 718 may be a conduit type component or portion extending from the main body 704. The exhaust lumen 722 is fluidly connected or coupled to the main lumen at the wall region of the main body via an opening(s) or a port(s) indicated generally at 724 formed in the wall of the main body for or by the protruding vent 718 formation.

In the embodiment shown in FIG. 31, the protruding vent 718 comprises a single exhaust lumen 722. In alternative embodiments, a plurality or multiple lumens may extend along the length of the protruding vent 718. For example, an array or mesh arrangement of multiple lumens may be provided within the protruding vent.

In this embodiment, the protruding vent 718 is a cylindrical component or has a substantially circular cross-section along its length between its connecting end at the wall of the main body 704 and its terminating end at opening 720. However, it will be appreciated that the cross-sectional shape or profile may be varied as desired, in alternative embodiments.

The internal diameter or dimension of the protruding vent 718 may be substantially similar to the inner diameter or dimensions of the main body, or alternatively may be smaller as shown.

In this embodiment, the protruding vent 718 extends from the main body 704 at an angle. Referring to FIG. 31, in this embodiment the main body 704 of the measurement device 702 is a substantially straight component. The main body 704 is defined by or relative to a longitudinal axis BB extending centrally through the main body. In this embodiment, the protruding vent 718 may be defined relative to an associated longitudinal axis CC extending through its center. In this embodiment, the protruding vent 718 extends at an angle indicated at 726, which is the angle between the relative longitudinal axes BB and CC of the main body 704 and protruding vent 718 respectively. In this embodiment, the angle 726 or angular direction of the protruding vent 718 may be acute in a direction toward the mouthpiece end 708. In another embodiment, the protruding vent 718 may be configured to extend at an acute angle toward the connector end 706 of the main body. In another embodiment, the protruding vent may extend a perpendicular or normal angle relative to the main body, i.e angle 726 may be approximately 90 degrees.

In this embodiment, the protruding vent 718 is configured to direct any exhaled or exhaust gas from the measurement device away from the user's face during use. For example, in the embodiment shown, the measurement device 702 may be oriented during use such that the protruding vent 718 extends downward so as to direct any exhaust gases below the user's chin, away from their face, eyes and/or nose for example. In alternative configurations, the protruding vent 718 may alternatively be directed or angled toward the connector end, so as to direct any exhaust gases away from the user's face, regardless of the orientation of the protruding vent.

It will be appreciated that the length, shape, and angle of the protruding vent relative to the main body of the measurement device may vary in other configurations or alternative embodiments. The protruding vent may be substantially straight as shown. Alternatively, the protruding vent may be arcuate, curved, bent, or any other suitable shape. The protruding vent may have one or more arcs or bends in some embodiments, to re-direct the flow of exhaust gas in the required direction, such as away from the user's face in use.

In alternative embodiments, the exhaust opening or openings may not protrude from the wall of the main body. For example, one or more exhaust openings may be provided that are non-protruding or flush through the peripheral wall of the main body 704. For example, the flush exhaust openings may comprise holes or vents formed directly in the wall of the main body 704.

In general, the properties of the one or more exhaust openings, whether protruding or non-protruding, may be varied including, but not limited to, the length, shape, width, area, cross-sectional area, circumference, diameter, single or multiple lumens, for example.

In one configuration, one or more of the exhaust openings may be single vents comprising a single opening. In another configuration, the one or more of the exhaust openings may be vents comprising a plurality of or multiple openings. In another configuration, the one or more of the exhaust openings may comprise vents having multiple openings in the form of a honeycomb or mesh structure of arrangement. It will be appreciated that various openings and vent configurations are possible.

In some configurations, the measurement device 702 may comprise a mixture of different types of exhaust openings. Some exhaust openings may be protruding vents, and others may be flush vents. Some exhaust openings may comprise a single opening, and others may comprise multiple openings, or may comprise an arrangement of openings or ports or holes forming a mesh or honeycomb vent arrangement, for example.

In the embodiment shown, a single exhaust opening 716 in the form of a protruding vent 718 is provided on one side surface of the main body 704 of the measurement device 702. In other embodiments, a plurality of exhaust openings of any type may be provided on one or more sides or surfaces of the main body 704. In some embodiments, exhaust openings may be provided on opposite or opposing sides, or on multiple sides of the main body 704. In some embodiments, a plurality of spaced-apart exhaust openings may be provided in a line or lines along the length of the main body 704 on one or more sides. In yet other embodiments, the exhaust opening may circumscribe the circumference of the main body 704. For example, the exhaust opening may be in the form of an annular vent formed about the circumference of the main body. The annular vent may comprise an arrangement of multiple holes or apertures through the wall of the main body 704.

The location of the one or more exhaust openings along the length of the main body 704 may vary. In one configuration, the exhaust opening or openings may be centrally located or located in a middle region of the measurement device. In other configurations, the exhaust opening or openings may be located at or toward either the connector end 706 or mouthpiece end 708 of the main body. In yet other configurations, a plurality of exhaust openings may be spaced-apart along the bulk or entire length of the main body 704, whether on the same or different side surfaces of the main body.

### Removable Mouthpiece

As discussed, in some embodiments the mouthpiece end 708 of the main body may itself be a mouthpiece, or may have an integrated mouthpiece. In this embodiment, the measurement device is provided with a releasable or removable mouthpiece that is received or attached to or into the mouthpiece end 708 of the main body 704.

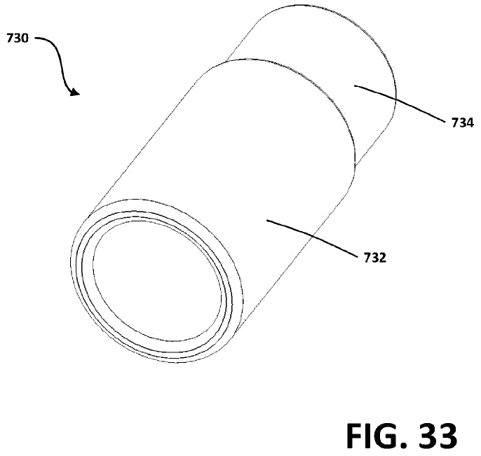

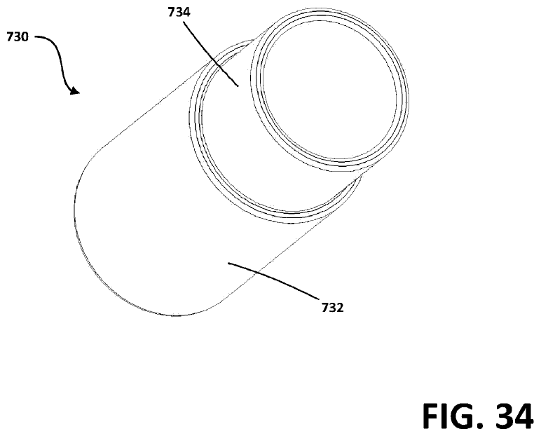

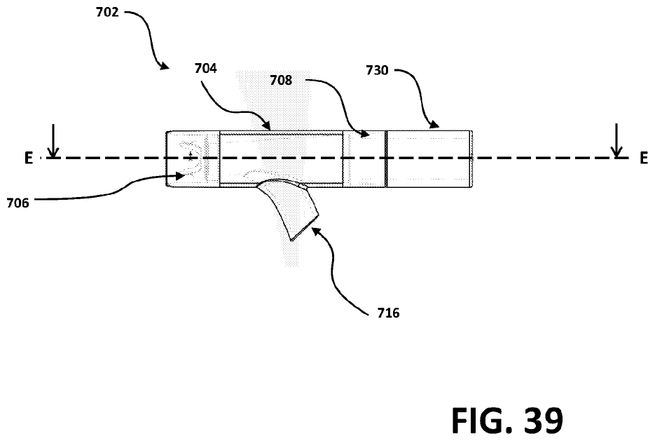

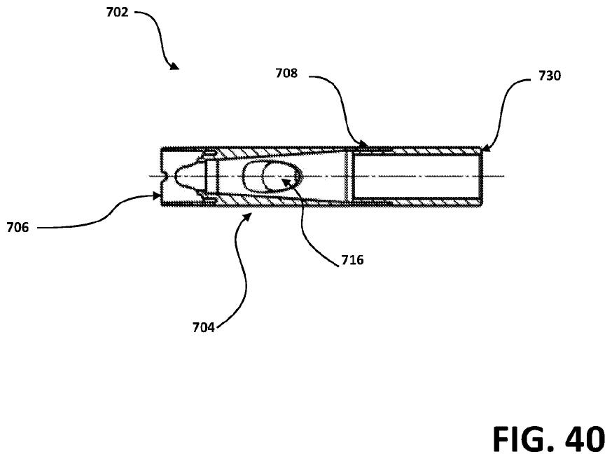

With reference to FIGS. 33-41, an embodiment of the measurement device 702 with a removable mouthpiece 730 will be described in further detail, by way of example. The mouthpiece 730 may be optional, in the sense that the mouthpiece end 708 of the main body 704 of the measurement device can operate as a mouthpiece. However, the removable mouthpiece 730 may provide certain advantages in some scenarios or circumstances from a usability, manufacturing and/or hygiene viewpoints.

In this embodiment, the removable mouthpiece 730 is a cylindrical conduit or tubular component with a mouthpiece portion 732 for the user's mouth at one end, and an attachment portion 734 at the other end for releasably connecting or attaching to the mouthpiece end 708 of the measurement device 702.

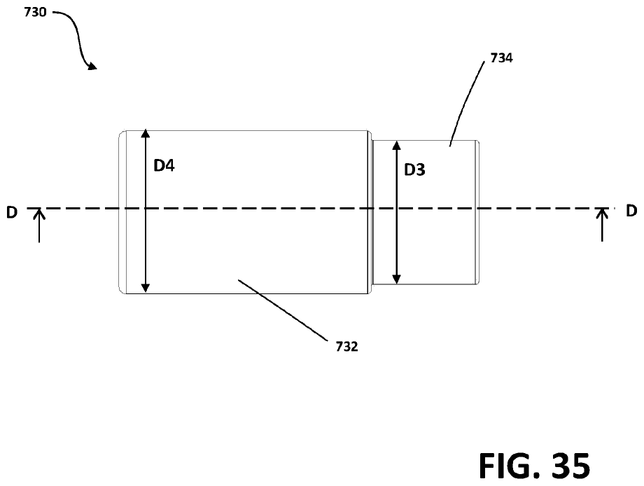

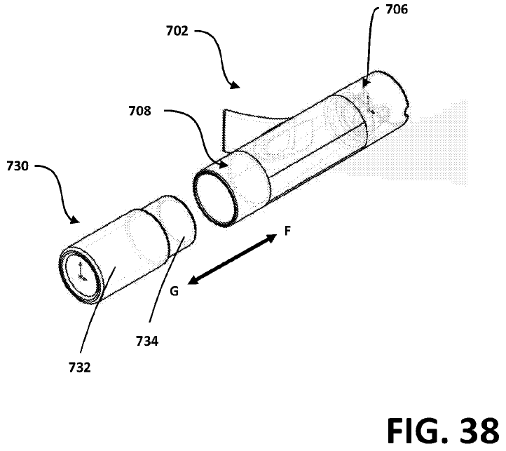

Referring to FIG. 35, in this embodiment the attachment portion 734 of the mouthpiece 730 is of reduced or smaller external diameter D3 or circumference relative to the diameter D4 or circumference of the mouthpiece portion 732. In this embodiment, the diameter D4 of the mouthpiece portion 732 of the mouthpiece 730 is substantially similar or the same as the diameter of the main body 704 of the measurement device. The reduced diameter D3 of the attachment portion 734 is dimensioned or selected to complement the internal diameter D2 of the mouthpiece end 708 of the main body (see FIG. 31) such that the attachment portion 734 can be inserted or plugged into the socket or port or opening provided by the mouthpiece end 708, as shown by arrow F in FIG. 38. In this embodiment, the relative dimensions of the outer diameter or circumference of the attachment portion 734 of the mouthpiece and the inner diameter of the mouthpiece end 708 of the main body 704 of the measurement device may be configured to provide a push-fit, friction fit or interference fit, so that the removable mouthpiece can be suitably received and retained in the measurement device during use. Sufficient hand or pulling force in direction G (see FIG. 38) may be applied by a user to remove or pull the removable mouthpiece 730 from the mouthpiece end 708 of the measurement device, for replacement, cleaning, repair and/or disposal for example.

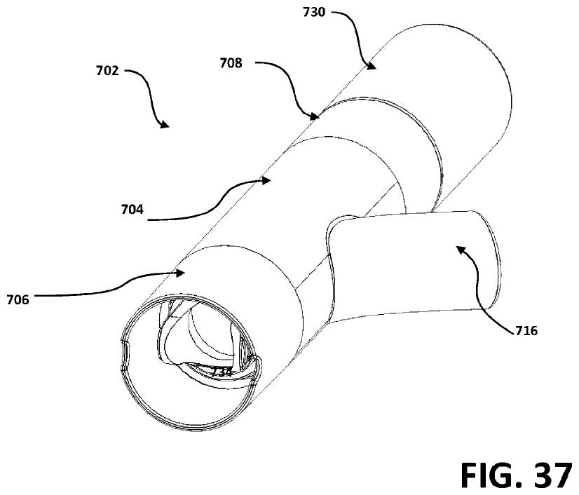

Referring to FIGS. 37-41, the assembly and disassembled measurement device 702 with removable mouthpiece 730 is shown by way of example. FIG. 38 shows an exploded view of the measurement device 702 with the mouthpiece 730 removed or disconnected from the mouthpiece end 708. FIGS. 37 and 39-41 show various perspective, elevation and cross-sectional views of the measurement device 702 with the removable mouthpiece installed, assembled or inserted into the mouthpiece end 708 of the main body 704, ready for use.

It will be appreciated that any other suitable releasable or removable coupling or connection arrangement or structure for connecting the removable mouthpiece 730 to the end of the measurement device 702 may be used in alternative embodiments including, but not limited to, snap-fit, screw-thread, fastening or clipping systems, or the like.

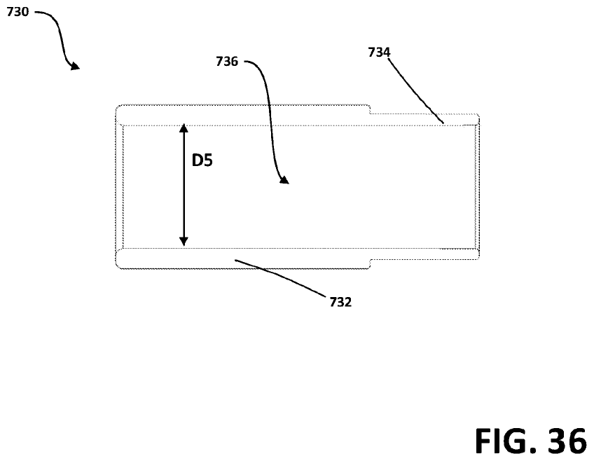

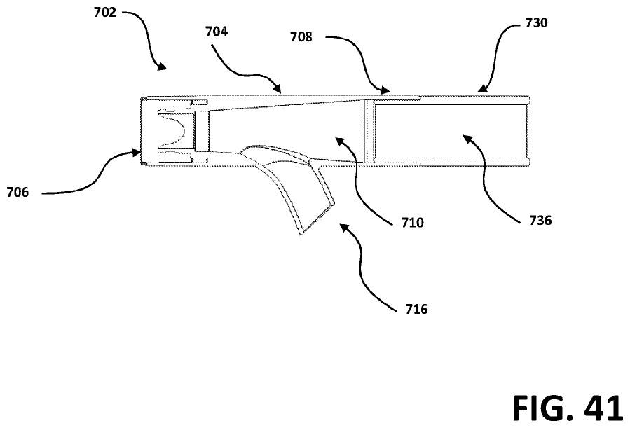

Referring to the cross-sectional view in FIG. 36, the removable mouthpiece in this embodiment comprises a single main lumen or passage indicated at 736 extending between its ends. Referring to FIG. 41, the mouthpiece lumen 736 provides the fluid communication to the main lumen or lumens (e.g. main lumen 710) of the main body 704 of the measurement device, so that the removable mouthpiece can convey the flow of gases to a user during use. In this embodiment, the inner diameter D5 of the mouthpiece is uniform along its length. As such, the peripheral wall defining the mouthpiece 730 has a reduced thickness in the attachment region 734 relative to the mouthpiece portion 732, and this creates the reduced external diameter D3 of the attachment portion 734 relative to the mouthpiece portion 732 (shown in FIG. 35).

In alternative embodiments, the mouthpiece may be provided with a plurality or multiple lumens (e.g. channels or passages) extending along the length of the mouthpiece, e.g. in an array or mesh or honeycomb type arrangement.

The removable mouthpiece 730 in this embodiment is elongate and cylindrical with a circular cross-sectional shape along its length. However, it will be appreciated that the cross-sectional shape may be non-uniform in other embodiments. For example, the shape of the attachment portion 734 may complement or suit the mouthpiece end 708 of the measurement device 702, and the mouthpiece portion 732 may be any other suitable shape or profile for a user to seal their mouth around or over. For example, the mouthpiece portion 732 may be circular, elliptical, oval or any other suitable shape in cross-section.

The removable mouthpiece 730 in this embodiment is a substantially straight component. However, it will be appreciated that the mouthpiece may have any suitable shape, including having one or more arcs or bends, or may be elbow-like in general shape.

As discussed, in this embodiment the removable mouthpiece 730 is arranged to be a releasable component of the measurement device 702. However, the mouthpiece described above may alternatively be integrally formed or extend from the main body 704 of the measurement 702 in other embodiments. It will be appreciated that any of the properties and aspects of the removable mouthpiece described above may equally apply to an integral or permanent mouthpiece portion of the main body 704.

### 3.2. Second Example Embodiment—Mechanical Configuration of Measurement Device

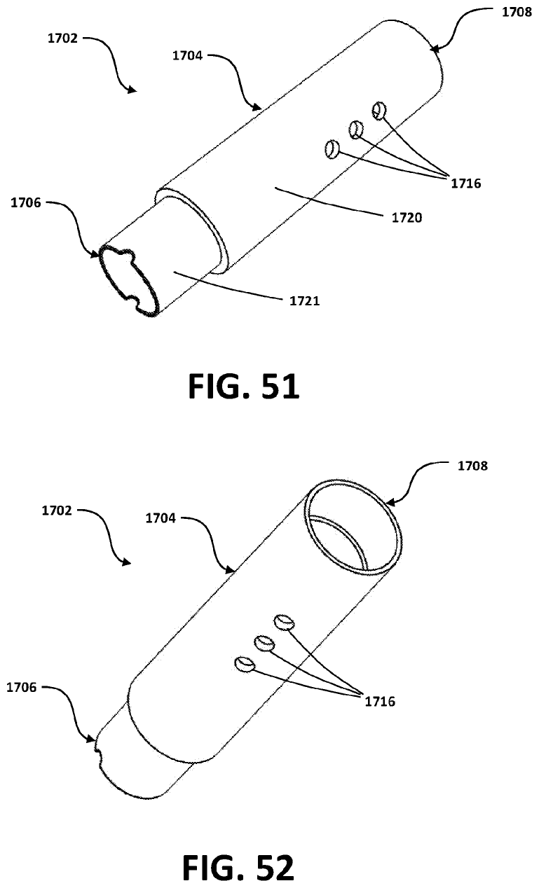

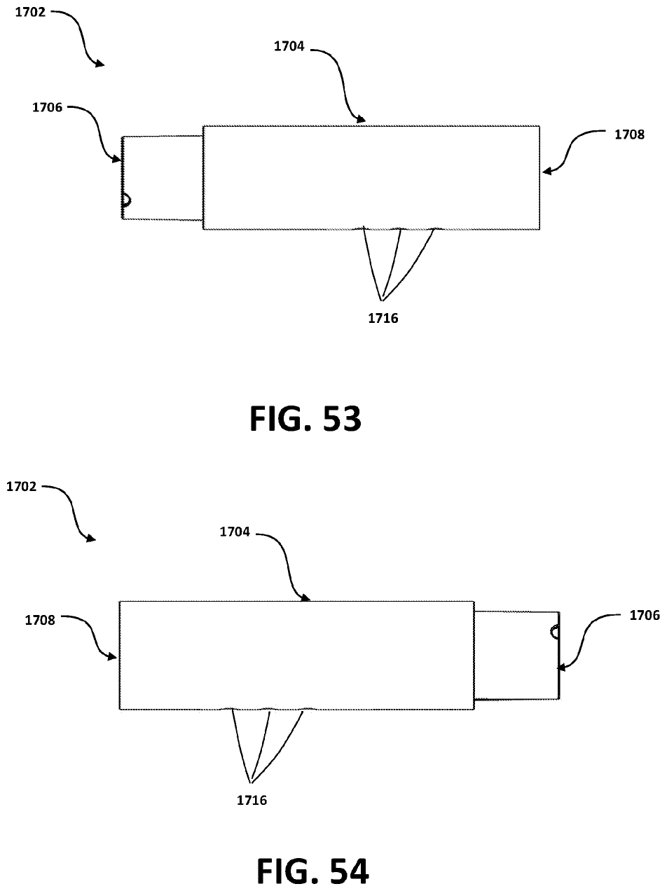

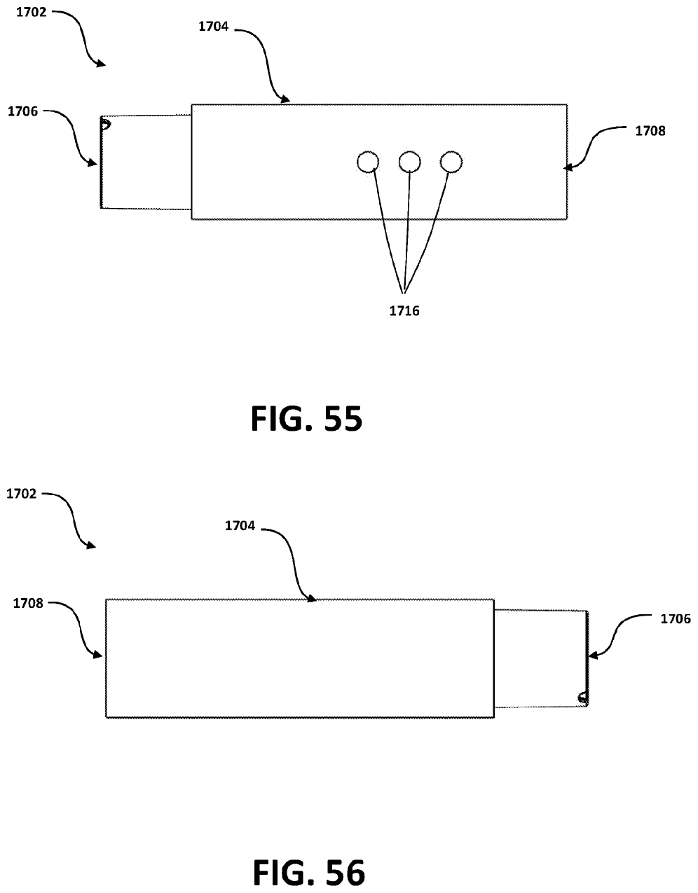

Referring to FIGS. 51-60, a second example embodiment of the measurement device 1702 will be described in further detail. This second example embodiment of the measurement device 1702 may be used in a similar manner to the first example embodiment of the measurement device 702. For example, reverting to FIG. 22, measurement device 1702 may be attached or connected to a breathing assistance apparatus 10 in the same manner as measurement device 702, to provide a similar measurement system 700. The overall configuration of the example measurement devices 702 and 1702 is similar, but the differences will be highlighted in the following description. It will be appreciated that the alternatives or variations described in regard to either example embodiment may apply to both embodiments.

In this embodiment, the measurement device 1702 comprises a main body 1704 that extends between a first end 1706 and a second end 1708. The first end 1706 of the main body is a connector end, and the second end 1708 is a mouthpiece end.

In this embodiment, the main body 1704 is a component in the form of a conduit, tube or tubular component, or manifold component. The main body has a main lumen or lumens extending between its connector end 1706 and mouthpiece end 1708 to allow a flow of gases to flow or be conveyed along the main body 1704 between its ends. The main lumen or lumens are in the form of a passage or channel or internal space extending between the openings at the connector end 1706 and mouthpiece end 1708.

### Main Body and Main Lumen

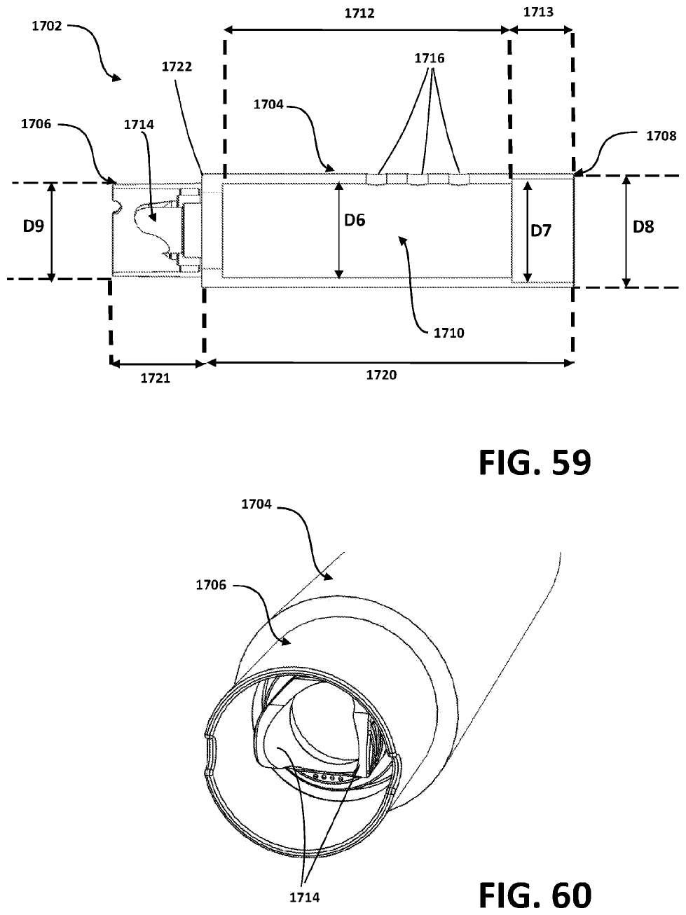

Referring to FIG. 59, in this embodiment, the main body 1704 comprises a single main lumen generally indicated at 1710. The main lumen 1710 is a passage or channel extending along the length of the main body 1704 between the open ends 1706, 1708 of the main body. The shape and/or dimensions of the main lumen 1710 may be uniform or non-uniform along the length of the main body 1704. For example, the inner dimensions or diameter of the main lumen 1710 may be uniform or vary along the length of the main body 1704. The main lumen 1710 is generally defined by the surrounding peripheral wall of the main body and/or any internal features within the main body.

In this embodiment, the internal diameter of the main lumen 1710 is substantially uniform along at least a portion of the length of the main body 1704. Referring to FIG. 59, a central portion of the main lumen, generally indicated at 1712 between the connector end 1706 and mouthpiece end 1708, has a substantially uniform diameter, as indicated by D6. In this embodiment, the internal diameter D7 of the main lumen 1710 widens or is larger in a mouthpiece end region 1713 at or toward the mouthpiece end 1708, relative to internal diameter D6 in the central region 1712 of the main lumen 1710. In this example embodiment, the internal diameter D6 of the main lumen in the central region 1712 steps or transitions into the larger diameter D7 in the mouthpiece end region 1713.

In one example, the internal dimension or diameter D6 in the central region 1712 of the main lumen 1710 may be approximately 22.5 mm and the internal dimension or diameter D7 in the mouthpiece end region 1713 may be approximately 25 mm, but it will be appreciated alternative dimensions or diameters may be used, depending on the characteristics of the breathing conduit for connection.

This configuration can also be described as the central portion 1712 of the main lumen 1710 having a substantially uniform cross-sectional area. In other embodiments, it will be appreciated that the internal profile, cross-sectional area or diameter of the main lumen 1710 may vary in alternative arrangements or configurations, including widening, narrowing, or a combination of these along one or more portions or the entire length of the main body 704. The variations or transitions in diameter or profile or cross-sectional area of the main lumen 1710 may be gradual or progressive, or sharper or with stepped changes.

In this embodiment, the difference in the internal diameters, dimensions or cross-sectional areas of the central region 1712 and mouthpiece end region 1713 are defined by the thickness of the peripheral wall of the main body 1704. In this embodiment, the outer dimension or diameter of the main body 1704 of the measurement device 1712 is substantially uniform along the central 1712 and mouthpiece end regions 1713, as indicated by diameter D8. As such, the thickness of the peripheral wall of the main body 1704 in the central region 1712 is greater than the thickness of the peripheral wall of the main body in the mouthpiece end region 1713, to thereby create the internal diameters D6 and D7 discussed above.

In this embodiment, the main body 1704 is an elongate component. The main body 1704 is substantially hollow and defined by a conduit or conduits, or peripheral wall or walls, that extend between the ends 1706, 1708 of the main body. In this embodiment, the main body 1704 has a substantially circular cross-section along its length as shown in FIGS. 51, 52, 57 and 58 for example.

In one configuration, the outer dimensions or diameter of the main body 1704 may be substantially uniform along its length. In alternative configurations, the outer dimensions or diameter of the main body 1704 may vary along its length.

In this embodiment, the main body 1704 comprises a first region 1720 and a second region 1721, as shown in FIG. 59. The first region 1720 is cylindrical and defined by outer diameter or dimension indicated at D8. The second region 1721 is cylindrical and defined by outer diameter or dimension indicated at D9. In this example, the first region 1720 extends from the mouthpiece end 1708 and terminates at an intermediate location 1722 toward the connector end 1706, and the second region 1721 extends from the intermediate location 1722 to the connector end 1706. In this example, the first region 1720 includes the mouthpiece end region 1713 and central region 1712 of the main body 1704, and the second region 1721 includes or defines the connector end region.

In this embodiment, the first region 1720 is larger in dimension or diameter D8 than the dimension or diameter D9 of the second region 1721. For example, in this embodiment, the connector end region indicated at 1721 has a smaller diameter or dimension than the remainder of the main body 1704. In this embodiment, the first region 1720 of the main body 1704 is of larger diameter or dimension D8 and steps down or transitions to the second region 1721 of the main body that has a smaller diameter or dimension D9. In this embodiment, the cylinder or cylindrical wall defining the first region 1720 steps down or transitions at intermediate location or shoulder 1722 to a cylinder or cylindrical wall defining the second region 1721. In this embodiment, the cylinder or cylindrical wall defining the first region 1720 is longer and larger in diameter than the cylinder or cylindrical wall defining the second region 1721. In this embodiment, the dimensions and configuration of the connector end region of the second region 1721 may be configured for complementary engagement or connection to the end of a breathing conduit 16 of a breathing assistance apparatus 10, as shown in FIG. 22.

In one example, the outer dimension or diameter D8 of the first region 1720 of the main body 1704 may be approximately 27.5 mm and the dimension or diameter D9 of the second region 1721 defining the connector end region may be approximately 20.8 mm, but it will be appreciated alternative dimensions or diameters may be used, depending on the characteristics of the breathing conduit for connection and/or other design factors.

In this embodiment, the main body 1704 has a substantially cylindrical form-factor or shape, with an outer surface defined by circular cross-sectional profile or shape along its length. It will be appreciated that the main body 1704 may be provided in alternative shapes or configurations in other embodiments. For example, the cross-sectional profile of the outer surface of the main body may be circular, oval, rectangular, square, arbitrary or any suitable shape or combination of shapes and sizes along the length of the main body.

In this embodiment, the main body 1704 and main lumen 1710 can generally be defined or aligned along or with reference to a central longitudinal axis. The main body and main lumen are straight and extend in a single axis or dimension. In alternative embodiments, it will be appreciated that the main body and/or main lumen may have alternative shapes and configurations such as, but not limited to, curvilinear, arcuate, elbow-configuration, or may otherwise have a non-straight profile that does not conform or align to a single longitudinal axis or dimension. It will be appreciated that various shapes and configurations of the main body can be provided with a main lumen that fluidly communicates between the connector end and mouthpiece end of the main body.

### Connector End

Referring to FIGS. 22 and 58-60, the connector end 1706 of the measurement device 1702 will be described in more detail.

In this embodiment, the connector end 1706 is configured to releasably connect or attach to the end of a breathing conduit 16 or tube of the breathing assistance apparatus 10. As will be appreciated, the breathing conduit 16 of a breathing assistance apparatus is a typically a flexible conduit that attaches or connects at one end to a gases outlet 21 of the breathing assistance apparatus such that it is fluidly connected or in fluid communication with the flow of gases generated by the flow generator 11 of the apparatus. The other end of the breathing conduit 16 typically provides a connector for releasably connecting or coupling to a patient interface (e.g. nasal canular, nasal mask, full face mask, tracheostomy interface or similar) to deliver the flow of gases to a patient's airway when the breathing assistance apparatus is used for respiratory therapy (e.g. high flow therapy, PAP therapy or similar), e.g. in normal therapy mode. In this embodiment, the connector end 1706 of the measurement device 1702 is configured or arranged to releasably connect or attach to the connector or end of the breathing conduit such that the measurement device is in fluid communication with the flow of gases conveyed along the breathing conduit.

In this embodiment, the connector end 1706 of the measurement device 1702 comprises a connecting structure or arrangement generally indicated at 1714 that is configured to provide a releasable fluid connection to the end or connector of a breathing conduit 16.

The end or connector of the breathing conduit may comprise a complementary connecting structure or arrangement for engaging and attaching with the connector end 1706 of the measurement device 1702 for coupling the two components together in a releasable manner. In this embodiment, the connecting structure 1714 may be provided by a pair of opposing resilient clipping protrusions that releasably engage or clip into corresponding formations or indents or catches or complementary end or connector of the breathing conduit 16. It will be appreciated that the connector end 1706 may be configured, arranged or provided with any suitable form of mechanical releasably fastening or coupling to complement the end or a connector of the breathing conduit including, but not limited to, screw threaded, rotational locking or coupling, clip fittings, snap-fit connection, push-fit connection, interference-fit connection, latch connection, or the like.

In some embodiments, the connector end 1706 may be provided with a connecting structure that is compatible with connecting to the end of one or more specific types of breathing conduits, including brand or manufacturer-specific breathing conduits. For example, in one embodiment, the connector end 1706 may be configured to attach or connect to a 20 mm breathing tube used with a high-flow breathing assistance apparatus. In other embodiments, the connector end 1706 may be provided with a generic or universal connecting structure or configuration that is operable or able to couple to the end of various or a broad range of different types of breathing conduits.

In the embodiment shown, the connector end 1706 is configured for releasable connection or coupling with the end or a connector of the breathing conduit 16. This allows the measurement device 1702 to be connected to the breathing conduit to perform patient diagnosis measurements in patient diagnosis mode of the apparatus, and then removed following the measurements, leaving the breathing conduit able to be connected or re-connected to a patient interface for normal respiratory therapy mode.

In alternative embodiments, the connector end 1706 of the measurement device may be configured with a non-releasable permanent connection to the breathing tube 16, such that the components can't be released from each other without breakage. Alternatively, a semi-permanent connection may be provided between the connector end 1706 of the measurement device and the breathing conduit 16, such that tools or similar are required to release the components from each other.

In further alternative embodiments, the measurement device 1702 may be provided with an integral breathing conduit extending from the connector end 1706. In such an embodiment, the measurement device 1702 is combined with an integral flexible conduit extending from the connector end. The integral flexible conduit may terminate in a connector end which can releasably connect or attach to the gases outlet 21 of the breathing assistance apparatus.

In another embodiment, the measurement device 1702 may be configured to releasably connect or attach directly to the gases outlet 16 of the breathing assistance apparatus without an intermediate breathing conduit. In another embodiment, the measurement device 1702 may be configured to releasably connect or attach directly to the flow generator outlet or gases outlet port 322, if the humidifier chamber is removed from the apparatus. In another embodiment, the humidifier or humidifier chamber may be bypassed via a bypass conduit or other bypass configuration. For example, the measurement device may attach directly to or indirectly via the breathing conduit to a bypass conduit or port or outlet that bypasses the humidifier or humidifier chamber, such that the measurement device is in fluid communication with the flow of gases from the flow generator outlet, and the humidifier is temporarily cut-out or bypassed from the flow path.

### Mouthpiece End

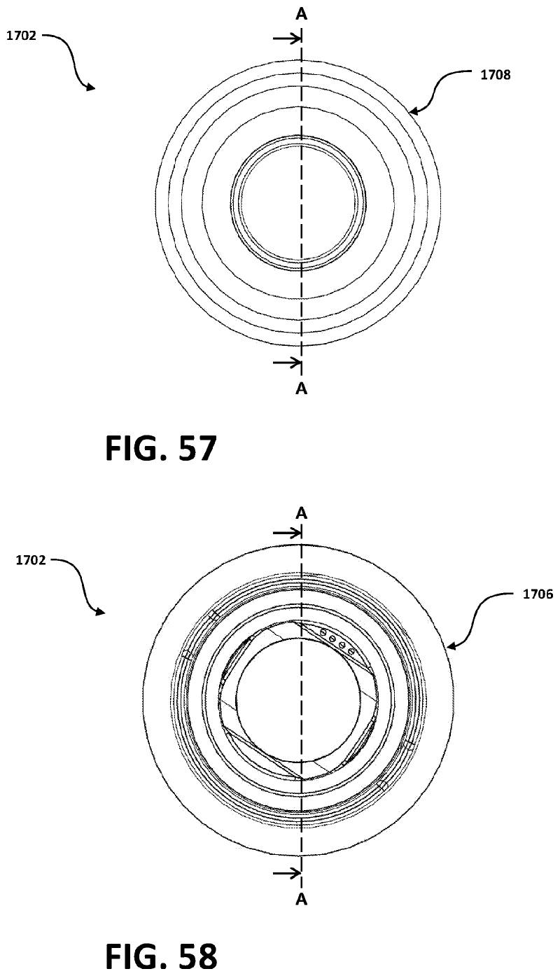

Referring to FIGS. 57 and 59, the mouthpiece end 1708 of the measurement device 1702 will be described in more detail.

In this embodiment, the mouthpiece end 1708 is a portion that is configured to releasably receive and retain an optional removable mouthpiece, to be explained in further detail later. In this embodiment, the mouthpiece end 1708 is an open cylindrical portion at the end of the main body 1704. The mouthpiece end 1708 is non-threaded in this embodiment, but could be threaded in other embodiments. However, it will be appreciated that the mouthpiece end 1708 may be any other suitable shape or configuration for receiving and retaining a releasably mouthpiece.

In alternative embodiments, the measurement device 1702 may be used directly without a separate removable mouthpiece. In such embodiments, the mouthpiece may be integrally formed with the mouthpiece end 1708 or the mouthpiece end 1708 itself may be configured as a mouthpiece or used as a mouthpiece for fluidly communicating with the airway of a user or patient when they seal their mouth around the mouthpiece, in use. It will be appreciated that the shape and configuration of the mouthpiece portion or end of the main body may be cylindrical, oval, elliptical, mouth-shaped or any other suitable shape suitable for a user's mouth.

### Exhaust Vent Holes

In this embodiment, the measurement device 1702 comprises one or more exhaust openings that are in fluid communication with the main lumen of the measurement device 1702. The exhaust opening or openings provide a pathway for the flow of gases and/or exhaled breath from the user or patient to escape to atmosphere or the surrounding environment, during use of the measurement device. Without one or more exhaust openings, complete blockage of the flow path may occur in some system configurations when the user places their mouth on the mouthpiece or otherwise creates a seal around the mouthpiece during use. In alternative embodiments, the measurement device 1702 may not have any exhaust vents or exhaust openings, and the system configuration may allow for this. For example, exhaust vents, exhaust openings, and/or pressure relief valves may be provided in the flow path upstream of the measurement device.

Referring to FIGS. 51-55 and 59, an example configuration of the exhaust openings will be described in further detail for this embodiment. In the embodiment shown, the measurement device 1702 is provided with one or more exhaust openings 1716. The one or more exhaust openings 1716 are located between the connector end 706 and mouthpiece end 708. In this embodiment, the one or more exhaust openings 1716 are provided in and/or along the main body 704 of the measurement device 1702.

In this embodiment, the exhaust openings 1716 are in the form of or comprise flush vents. The flush vents are through-holes, openings or apertures extending through the peripheral wall of the main body 1704 of the measurement device 1702. The flush vents may be substantially flush with the outer surface (e.g. cylindrical surface) of the main body 1704 of the measurement device 1702. Referring to FIG. 59, in this embodiment, the exhaust openings 1716 extend from the outer surface through to the inner surface of the wall of the main body (i.e. through the entire thickness of the wall) to thereby provide a passage(s) or pathway(s) for gases and/or exhaled breath in the main lumen to escape or exhaust from the device to the surrounding atmosphere or environment. As shown, in this embodiment, the exhaust openings are non-protruding or flush relative to the peripheral wall of the main body 1704 and comprise holes or vents formed directly in the wall of the main body.

In this embodiment, the shape of the exhaust openings 1716 is circular, but it will be appreciated that any other shape or profile or combination of different shapes or profiles may be used for the exhaust openings or holes in alternative embodiments. If a plurality of exhaust openings 1716 are provided, these may be uniform in shape and/or size, or alternatively they may be a combination of different shapes and/or sizes.

The total cross-sectional area of the exhaust opening or openings 1716 may be configured to suit the desired exhaust flow requirements. For example, the size or dimension of the exhaust openings 1716 may be configured to suit the exhaust flow requirements, relative to the size and characteristics of the overall measurement device 1702. For example, for circular exhaust openings 1716, the diameter of the openings may be customised or configured to suit the required exhaust flow requirements.

By way of example only, in this example embodiment, each of the exhaust openings 1716 is a circular hole or aperture having a diameter of approximately 5 mm. As such, the opening area of each exhaust opening is approximately 19.6 mm². In this example, the inner cross-sectional area of the main lumen is approximately 397.6 mm² as defined by the internal diameter D6 of 22.5 mm. As such, each exhaust opening 1716 has an opening area that is approximately 5% of the cross-sectional area of the main lumen of the main body of the measurement device 1702. Further, in this example, the total exhaust opening area as provided by the three uniform exhaust openings 1716 is approximately 15% of the cross-sectional area of the main lumen of the measurement device 1702.

In the embodiment shown, exhaust openings 1716 comprise a linear arrangement, line or line array of uniformly spaced-apart exhaust openings 1716 provided along one side surface or region of the main body 1704 of the measurement device 1702. In this example embodiment, the line array of exhaust openings 1716 extends in a direction aligned with the longitudinal axis of the main body 1704. In this example embodiment, there are three exhaust openings, but it will be appreciated that the number of exhaust openings 1716 in the array may be varied and that the spacing may be uniform or non-uniform in other embodiments. In this example embodiment, the line array of exhaust openings 1716 is located closer to the mouthpiece end 1708 of the main body 1704 than the connector end 1706, but alternatively the exhaust openings 1716 may be located centrally or closer to the connector end of the main body in other embodiments.

There may be a single exhaust opening or a plurality of exhaust openings. The number, arrangement or pattern of the one or more exhaust openings may vary in different embodiments. In some configurations, the exhaust openings are may arranged in a 1D array (e.g. line array as shown) or a 2D or multi-dimensional array, or any other pattern or configuration. In some configurations, the exhaust openings may be provided in a line or lines extending along the length of main body 1704 on one or more sides or surface regions. In some configurations, the line or lines of exhaust openings are aligned with the longitudinal axis as shown, or they may extend in other directions.

In some configurations, the exhaust openings may be provided circumferentially about the circumference of the main body. For example, the exhaust openings may be provided in the form of one or more an annular vents or annular arrays of spaced-apart exhaust openings about the circumference of the main body 1704. The annular array or arrays of spaced-apart exhaust openings or holes in the wall of the main body may extend about the entire circumference or at least a portion or portions of the circumference of the main body.

There may be one or a plurality of exhaust openings or arrays of exhaust openings extending along and/or about the surface of the main body 1704, including along or about any one or more regions or sides or surfaces of the main body. In some embodiments, the exhaust openings may be provided on opposite or opposing sides, or on multiple sides of the main body 1704.

The location of the one or more exhaust openings or arrays or exhaust openings along and/or about the main body 704 may vary. In one configuration, the exhaust opening or openings may be centrally located or located in a middle region of the measurement device 1702 or main body 1704. In other configurations, the exhaust opening or openings may be located at or toward either the connector end 1706 or mouthpiece end 1708 of the main body 1704. In yet other configurations, a plurality of exhaust openings may be spaced-apart along the bulk or entire length of the main body 704, whether on the same or different side surfaces of the main body.

In general, the properties of the one or more exhaust openings 1716 may be varied in different embodiments including, but not limited to, the number, shape, opening area, arrangement, uniformity, circumference, and/or diameter for example.

In some configurations, the measurement device 1702 may comprise a mixture of different types of exhaust openings. Some exhaust openings may be protruding vents of the type described in regard to the first example embodiment measurement device 702, and others may be flush vents or exhaust openings of the type described in regard to this second example embodiment measurement device 1702. Some exhaust openings may comprise a single opening, and others may comprise multiple openings, or may comprise an arrangement of openings or ports or holes forming a mesh or honeycomb vent arrangement that is flush with the outer surface of the main body, for example.

### Removable Mouthpiece

As discussed, in some embodiments the mouthpiece end 1708 of the main body 1704 may itself be a mouthpiece, or may have an integrated mouthpiece. In this embodiment, the measurement device 1702 is provided with a releasable or removable mouthpiece that is received or attached to or into the mouthpiece end 1708 of the main body 1704.

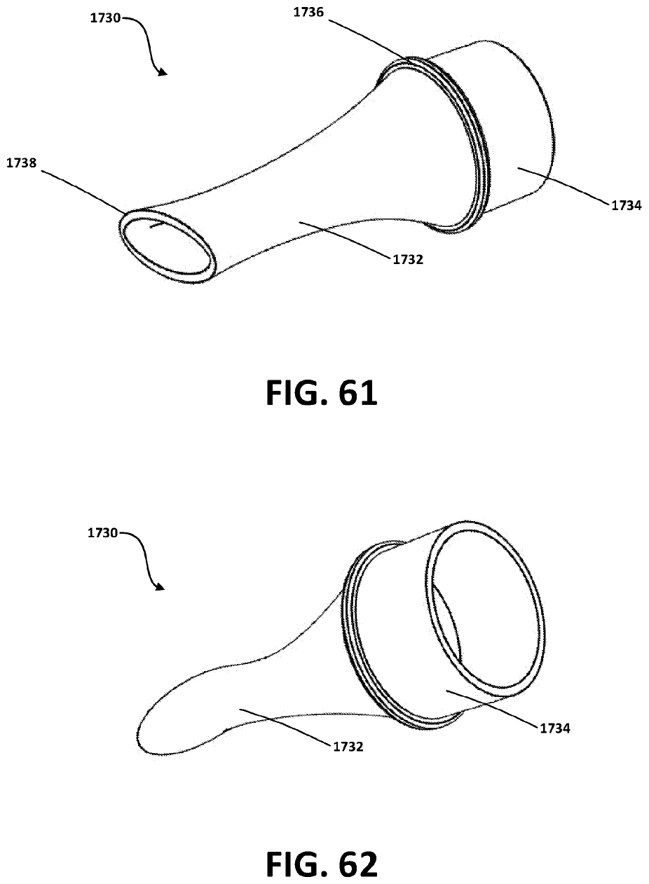

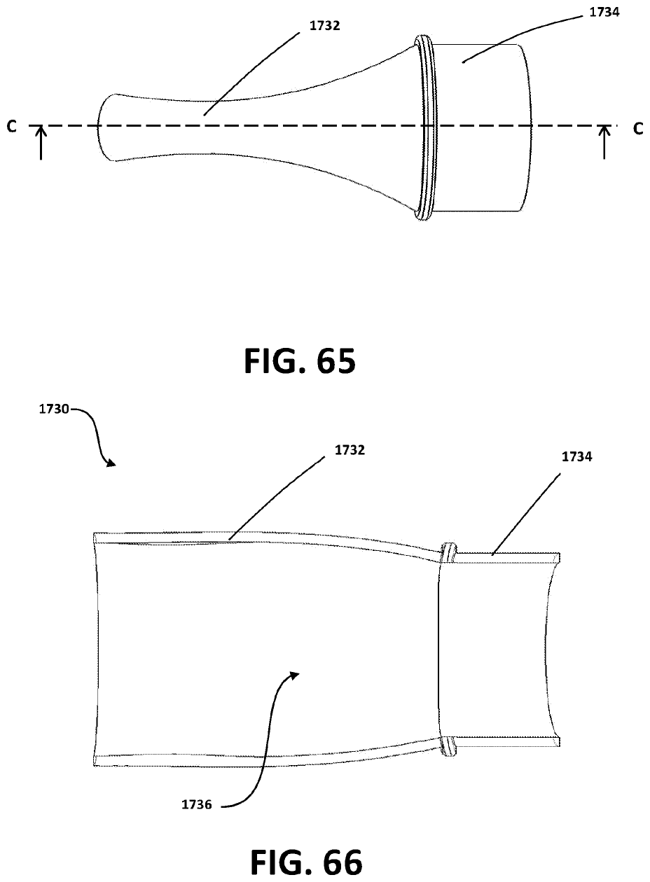

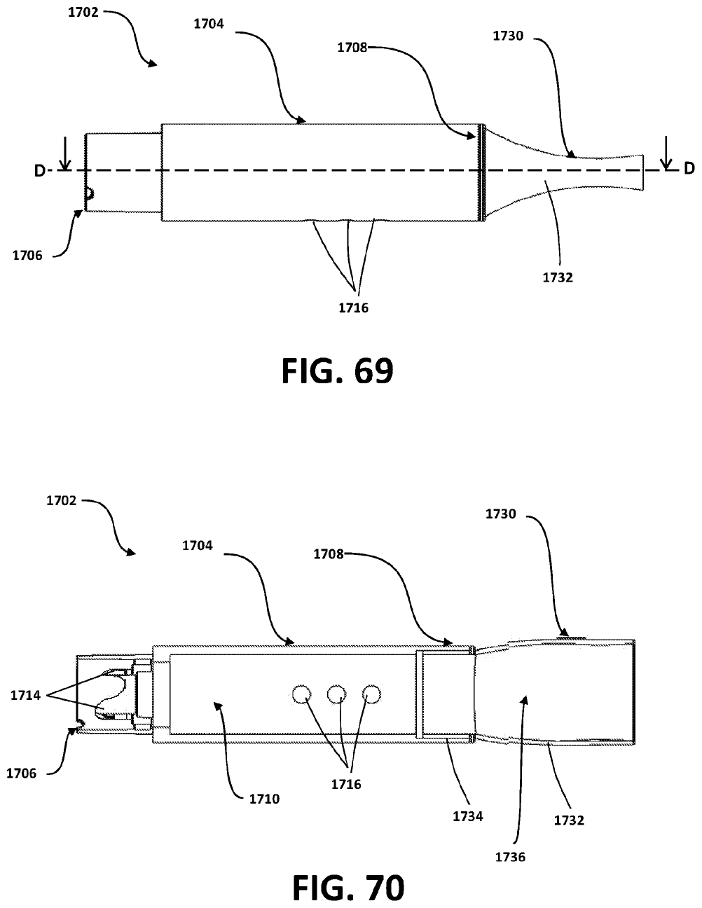

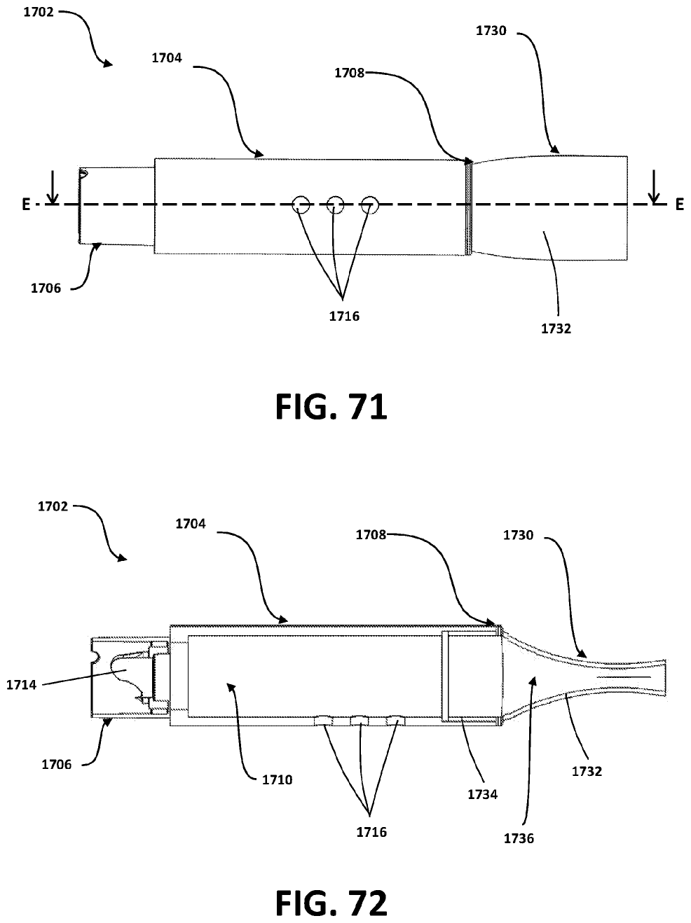

With reference to FIGS. 61-72, an embodiment of the measurement device 1702 with a removable mouthpiece 1730 will be described in further detail, by way of example. The mouthpiece 1730 may be optional, in the sense that the mouthpiece end 1708 of the main body 1704 of the measurement device 1702 can operate as a mouthpiece. However, the removable mouthpiece 1730 may provide certain advantages in some scenarios or circumstances from a usability, manufacturing and/or hygiene viewpoints.

In this embodiment, the removable mouthpiece 730 is a hollow conduit component with a mouthpiece portion 1732 for the user's mouth at one end, and an attachment portion 1734 at the other end for releasably connecting or attaching to the mouthpiece end 1708 of the measurement device 1702.

In this example embodiment, the mouthpiece portion or region 1732 of the mouthpiece 1730 comprises an oval or elliptical shape or cross-sectional profile along at least a portion of its length. The mouthpiece portion or region extends between a first end or point indicated at 1736 and a second end or point indicated at 1738. The first end or point 1736 is at an intermediate location along the length of the mouthpiece 1730 and defines the boundary or transition between the mouthpiece portion 1732 and attachment portion 1734. In this example embodiment, the mouthpiece portion or region 1732 transitions progressively in shape or cross-section from a circular cross-section at the first end at 1736 to an oval or elliptical shape as it extends toward the open elliptical or oval opening at the second end 1738.

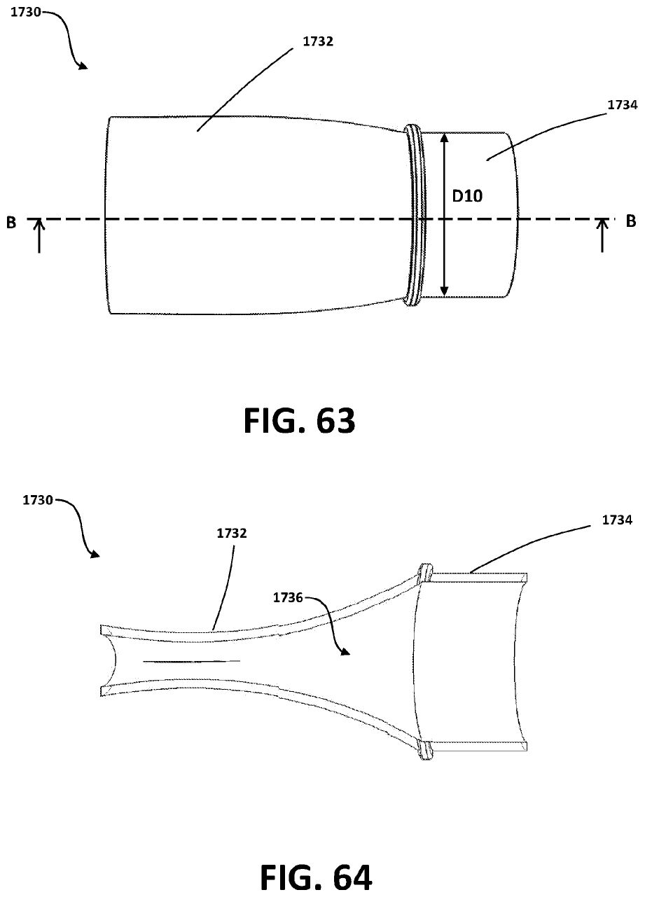

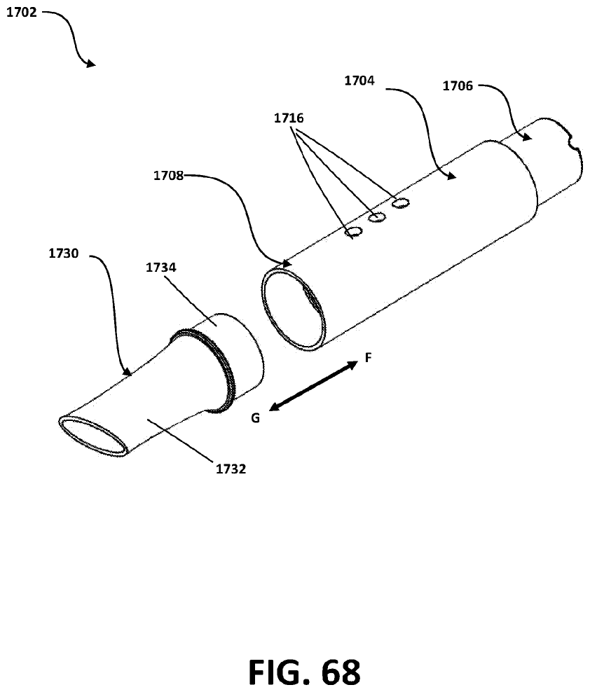

In this example embodiment, the attachment portion or region 1734 of the mouthpiece 1730 is a cylindrical conduit portion. Referring to FIG. 63, in this embodiment, the outer diameter D10 of the attachment portion 1734 of the mouthpiece 1730 is dimensioned or selected to complement the internal diameter D7 of the mouthpiece end 1708 of the main body (see FIG. 59) such that the attachment portion 1734 can be inserted or plugged into the socket or port or opening provided by mouthpiece end 1708, as shown by arrow F in FIG. 68. In this embodiment, the relative dimensions of the outer diameter D10 or circumference of the attachment portion 1734 of the mouthpiece 1730 and the inner diameter D7 of the mouthpiece end 1708 of the main body 1704 of the measurement device 1702 may be configured to provide a push-fit, friction fit or interference fit, so that the removable mouthpiece can be suitably received and retained in the measurement device during use. Sufficient hand or pulling force in direction G (see FIG. 68) may be applied by a user to remove or pull the removable mouthpiece 1730 from the mouthpiece end 1708 of the measurement device, for replacement, cleaning, repair and/or disposal for example.

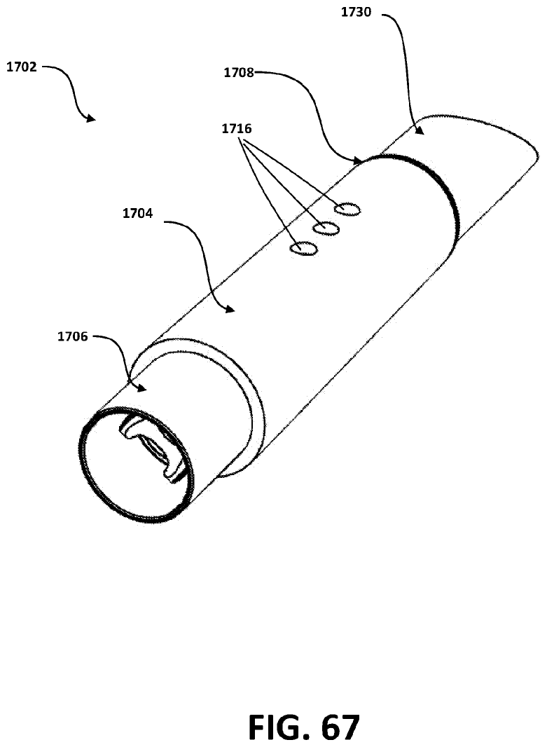

Referring to FIGS. 67-72, the assembly and disassembled measurement device 1702 with removable mouthpiece 1730 is shown by way of example. FIG. 68 shows an exploded view of the measurement device 1702 with the mouthpiece 1730 removed or disconnected from the mouthpiece end 1708. FIGS. 67 and 69-72 show various perspective, elevation and cross-sectional views of the measurement device 1702 with the removable mouthpiece 1730 installed, assembled or inserted into the mouthpiece end 1708 of the main body 1704, ready for use.

It will be appreciated that any other suitable releasable or removable coupling or connection arrangement or structure for connecting the removable mouthpiece 1730 to the end of the measurement device 1702 may be used in alternative embodiments including, but not limited to, snap-fit, screw-thread, fastening or clipping systems, or the like.

Referring to the cross-sectional views in FIGS. 64 and 66, the removable mouthpiece in this embodiment comprises a single main lumen or passage indicated at 1736 extending between its ends. Referring to FIGS. 70 and 72, the mouthpiece lumen 1736 provides the fluid communication to the main lumen or lumens (e.g. main lumen 1710) of the main body 1704 of the measurement device 1702, so that the removable mouthpiece can convey the flow of gases to a user during use.

In alternative embodiments, the mouthpiece may be provided with a plurality or multiple lumens (e.g. channels or passages) extending along its length, e.g. in an array or mesh or honeycomb type arrangement.

In this example embodiment, the removable mouthpiece 1730 is elongate and varies in cross-sectional shape along at least a portion of its length. It will be appreciated that various shapes or cross-sectional profiles may be used to create the mouthpiece 1730. In one example, the shape of the attachment portion 1734 may complement or suit the mouthpiece end 1708 of the measurement device 1702 to allow for attachment, and the mouthpiece portion 1732 may be any other suitable shape or profile for a user to seal their mouth around or over. For example, the mouthpiece portion 1732 may be circular, elliptical, oval, mouth-shaped, or any other suitable shape in cross-section.

The removable mouthpiece 1730 in this embodiment is a substantially straight or elongate component defined about a central longitudinal axis. However, it will be appreciated that the mouthpiece may have any suitable shape, including having one or more arcs or bends, and/or may be elbow-like in general shape.

As discussed, in this embodiment the removable mouthpiece 1730 is arranged to be a releasable component of the measurement device 1702. However, the mouthpiece 1730 described above may alternatively be integrally formed with or extend from the end of the main body 1704 of the measurement 1702 in other embodiments. It will be appreciated that any of the properties and aspects of the removable mouthpiece 1730 described above may equally apply to an integral or permanent mouthpiece portion of the main body 704.

### Anti-Occlusion Features

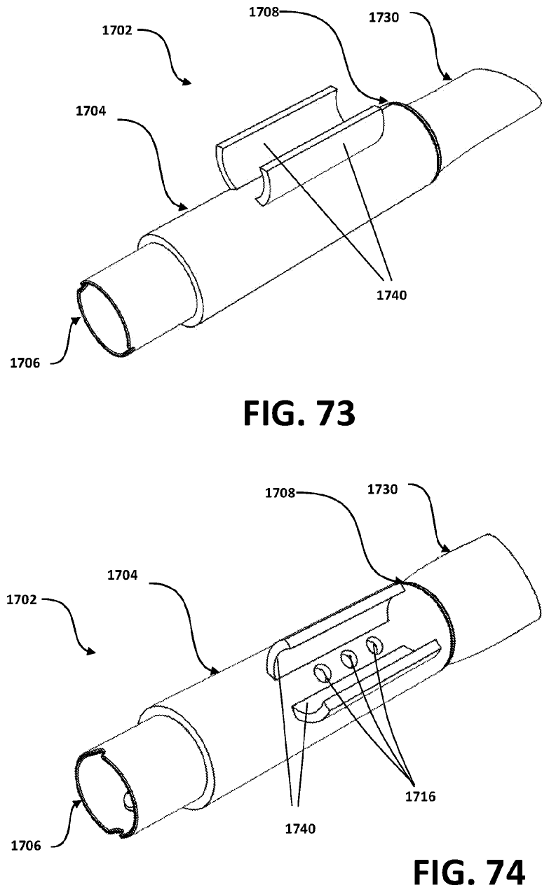

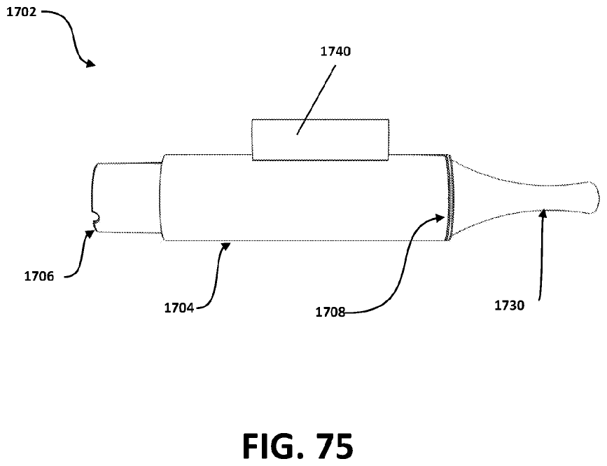

Referring to FIGS. 73-75, in some embodiments the measurement device 1702 may optionally comprise one or more anti-occlusion features, formations, or protrusions 1740 near, about or in the vicinity of the exhaust openings 1716 to assist in preventing the openings from being accidentally or inadvertently blocked or covered during use by a patient. For example, the anti-occlusion features 1740 may assist in preventing a user or patient from accidentally or inadvertently covering or blocking one or more of the flush exhaust openings 1716 with their fingers and/or hand in use.

In this example embodiment, the anti-occlusion features comprise a pair of spaced-apart walls or surfaces 1740 that protrude or extend from the wall of the main body 1704 along each side of the linear arrangement of exhaust openings 1716. The pair of walls protrude above the surface of the main body 1704 and exhaust openings 1716, and are arranged to prevent accidental or inadvertent blocking or covering of the exhaust openings in use.

In this example embodiment, the anti-occlusion protruding walls 1740 are curved or have an arcuate profile and are arranged to collectively form a clip or attachment mechanism that can be used to clip, attach or mount the measurement device 1702 to a carry stand or to the breathing assistance apparatus when not in use. For example, the pair of protruding curved walls 1740 oppose each other and form a cylindrical clipping aperture or region above the exhaust openings for clipping or mounting to a complementary sized and shaped cylindrical component of a carry stand or of the breathing assistance apparatus or some other complementary mounting structure or component. The protruding walls may be rigid or semi-rigid, and in some embodiments may have some resilient flexibility to enable provide or form a snap-fit clip or clipping arrangement.

It will be appreciated that the anti-occlusion features may be any other shape, size or arrangement relative to the exhaust openings that is sufficient to assist in preventing blockage or covering of the exhaust openings during use. There may be one anti-occlusion feature or a plurality or multiple anti-occlusion features. In some embodiments, the anti-occlusion features may have a dual purpose or function, such as also forming a clip, but in other embodiments the anti-occlusion features may have the sole function of preventing blockage of the exhaust openings during use.

### 3.3. Materials—Mechanical Configuration of Measurement Device

The example embodiments of the measurement device 702, 1702 described above may be formed of any suitable materials.

In one embodiment, the main body 704, 1704 may be primarily formed of a rigid or semi-rigid materials or combination of materials such as, but not limited to, plastic or plastic polymer such as, but not limited to, polycarbonate (PC), polyethylene (PE), polypropylene (PP), polyethylene terephthalate (PET), polyvinyl chloride (PVC), Acrylonitrile-Butadiene-Styrene (ABS), or carboards, glass, metal, or any other suitable rigid or semi-rigid material.

The main body 704, 1704 of the measurement device 702, 1702 may be made of the same or similar material to the removable mouthpiece 730, 1730, or these components may be formed of different materials. For example, in one embodiment, the main body 704, 1704 may be primarily formed of a plastic polymer and the removable mouthpiece may be formed of the same or different plastic polymer. In another embodiment, the main body 704, 1704 may be primarily formed of a plastic polymer, and the removable mouthpiece may be formed from cardboard or heavy-duty paper for example.

The materials used and/or thickness of the materials used to form the measurement device 702, 1702 may depend on the circumstances of use. In some embodiments, the entire measurement device, including the main body 704, 1704 and removable mouthpiece 730, 1730 may be made from lower-cost or lower-grade materials more suited for a disposable item. In other embodiments, the main body 704, 1704 may be configured as a multi-time use and/or multi-user component formed of a more durable or longer-lasting material such as plastic, but a lower-cost material may be used for removable mouthpiece, such as cardboard or a thinner or lower-grade or lower-cost plastic, as the mouthpiece may be configured as a one-time or single-user disposable item.

It will be appreciated that a range of different measurement devices may be formed from different materials. Some measurement devices, or particular components thereof, may be formed with higher-grade and/or more durable materials for longer or multi-time use, and other measurement devices, or at least particular components there of (e.g. the removable mouthpiece) may be formed of lower-cost and/or disposable and/or less durable single-use type materials such as, but not limited to, cardboard or lower-grade or thinner plastics.

In some embodiments, the measurement device or components thereof (e.g. removable mouthpiece) may be configured to be suited to delivery and/or shipping to end users, for use a specified number of times or for a fixed lifespan (e.g. having an expiry or use by date). In such scenarios, the measurement device may be formed of materials more suited to a disposable item.

### 3.4. Patient Diagnostic Mode of Breathing Assistance Apparatus

As discussed and shown in FIG. 22, the measurement device 702, 1702 is used with a breathing assistance apparatus 10 that comprises a flow generator that is operable to generate a flow of gases (e.g. atmospheric air or pressurized air, air augmented with oxygen or another supplementary gas, or any other suitable combination of gas or gases) and which has one or more sensors for sensing, measuring or detecting one or more properties or characteristics of the flow of gases. The measurement device 702, 1702 is provided as a supplementary component that can be selectively attached to the flow path of the breathing assistance apparatus in order to perform one or a range of lung performance or function measurements or assessments.

The breathing assistance apparatus may be provided with one or more modes of operation. In this embodiment, the breathing assistance apparatus have one or more therapy modes for providing a flow of gases with specific properties (e.g. flow rate and/or pressure controlled) suitable for high flow therapy, CPAP, bi-level PAP, or other such respiratory therapy. In this embodiment, the breathing assistance apparatus further comprises a patient diagnostic mode, which may be initiated or operated when the breathing assistance apparatus is used with the measurement device 702, 1702 to undertake lung performance measurements or assessments. In one example configuration, the breathing assistance apparatus may be operating in a first mode (e.g. a therapy mode) and may then switch to a second mode (e.g. a patient diagnostic mode or measurement mode or spirometry mode) when the measurement device 702, 1702 is attached for undertaking lung performance measurements. In another example configuration, the breathing assistance apparatus may be started in the second or patient diagnostic mode on start-up of the apparatus. In another example configuration, the apparatus may be provided as a dedicated measurement system or device having only a single mode, the single being a diagnostic or measurement mode, i.e. in this example configuration the apparatus may not provide any therapy modes.

As will be explained in further detail later, in the patient diagnostic mode, the user may be instructed or prompted to perform various lung performance manoeuvres (e.g. forced exhalation or expiration, or restful tidal breathing) into the mouthpiece of the measurement device 702, 1702 while the flow generator of the apparatus is controlled to generate a flow of gases to provide a pneumatic or air resistance against the user's forced exhalation(s) or restful tidal breathing. The one or more sensors of the apparatus 10 are used to sense or measure one or more properties or characteristics of the flow of gases during the lung performance manoeuvres. The sensor data may then be processed or filtered or otherwise analysed to generate data indicative of lung performance or lung function.

In this embodiment, the breathing assistance apparatus 10 may be configured to initiate or activate the patient diagnostic mode in response to either user input into the user interface of the apparatus 10 or the patient diagnostic mode may be triggered or activated remotely by another external device or system that is in data communication or connection with the apparatus 10 over a data link or data network.

By way of example, the user interface of the apparatus 10 may have an operable button (whether mechanical or touch-sensitive) or a touch-screen interface or button that is operable to trigger or start patient diagnostic mode. Additionally, or alternatively, the apparatus 10 may be controlled via a user's smart device in data communication with the apparatus (e.g., over Bluetooth, Wi-Fi, infrared or similar). For example, a smartphone application may be provided to remotely control the apparatus and the smartphone application may provide a button or graphical user interface that is operable to trigger the patient diagnostic mode on the apparatus 10.

Remote initiation or triggering of the patient diagnostic mode may occur by a remote user such as a clinician, healthcare provider, or physician providing user input to a remote electronic device or server that is in data communication with the breathing assistance apparatus 10. By way of example, the remote or external device or server may have a software application running which provides an operable command or graphical user interface (GUI) with a button or GUI element that is triggerable (e.g., via cursor click or touch-screen operation) to initiate the patient diagnostic mode on the apparatus 10 remotely via a control signal or command data sent over the data network. The remote device or server may have any form of suitable user interface for receiving user input from the clinician or the like, including a mechanical or touch-sensitive buttons or interfaces, or an electronic GUI displayed on a display screen within the context of a software application, as described.

It will be appreciated that the apparatus 10 and/or remote device or server or system may receive user input to trigger the patient diagnostic mode in other ways, including voice or audible control or commands via a voice interface or voice assistant device.

In an embodiment, the patient diagnostic mode may be initiated or triggered in response to manually via user input or control of the apparatus or remote actuation from a clinician or similar. Additionally, or alternatively, the patient diagnostic mode may be triggered automatically, either locally by the local controller 19 of the apparatus 10 or remotely by a processor of the remote device, system or server. Automatic triggering or actuation of the patient diagnostic mode may occur according to a configurable schedule or periodic interval. The configurable schedule or periodic interval may be configured by the user or by a remote clinician via the respective user interfaces and/or software applications of the apparatus 10 and remote device, system or server.

In an embodiment, the apparatus and/or remote device, system or server may be configured to prompt or remind the user or remote clinician to start or initiate the patient diagnostic mode. The prompts or reminders may be visual prompts and/or audio prompts for example via the user interfaces of the apparatus 10 and/or any remote device, server or system in data communication with the apparatus 10. By way of example, the visual prompts may be displayed on a display or touch-screen display of the apparatus 10 or remote device and any audible prompts may be provided via an audio output device or speaker of the apparatus or remote device. The user or remote clinician may trigger or initiate the patient diagnostic mode in response to the prompt or prompts. In some scenarios, the user may be prompted or contacted by mail, phone, email, SMS, or other communication method to prompt them to undertake a patient diagnostic method.

In some configurations, the prompts may be automatically generated in accordance with a preset or configurable schedule or a periodic interval. The apparatus may be provided with an initial default schedule or periodic interval during manufacture. The schedule or periodic interval may be configured by the user or remote clinician via an interface or software application of the apparatus and remote device, server or system. Alternatively, the remote clinician may be able to trigger the prompts on the apparatus 10 in a manual or ad hoc manner when desired via an interface or software application of the remote device, system or server.

### 3.5. Measurement Method and Process

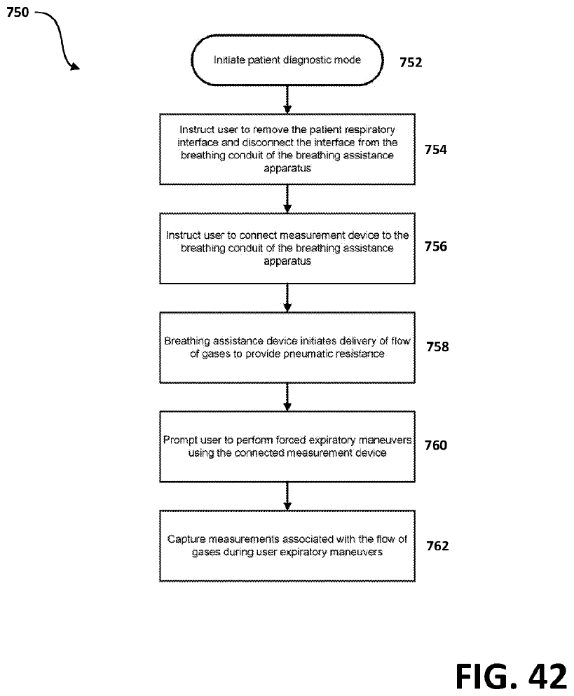

Referring to FIGS. 42-50, an example measurement or diagnostic methods or processes 750,750A of using the measurement device 702 with a breathing assistance apparatus 10 in patient diagnostic mode will be described in further detail. The patient diagnostic mode may also be referred to a measurement mode or spirometry mode or ‘spirometer check-in’. The measurement process or method in this example is implemented primarily by an algorithm or computing-instructions executed by a processor or controller of the breathing assistance apparatus 10 when it enters patient diagnostic mode. The principles of the example diagnostic methods or processes 750,750A may be applied to any of the examples, configurations, or variations of the breathing apparatus, measurement system and/or measurement device described above.

It will be appreciated that various steps in the example processes to be described need not necessarily operate in the order described. Some steps may occur in parallel or overlap or in a different order, without impacting on the overall process. Some steps may not be required or may be altered, depending on the specific configuration and/or operation of the breathing apparatus, measurement system and/or measurement device.

### Start Patient Diagnostic Mode

Referring to FIG. 42, in this embodiment, the diagnostic process 750 starts at 752 by the patient diagnostic mode of the breathing assistance apparatus 10 being triggered in response to manual actuation by the local user or patient, or remotely by a remote clinician or similar, or in response to automatic triggering. As previously discussed, the user or clinician may be prompted to start or initiate the patient diagnostic mode in response to a prompt or reminder. Once in the patient diagnostic mode, the breathing assistance apparatus 10 may be configured to provide the user with one or more prompts or guidance or instructions on some or all steps of the diagnostic assessment process, as will be explained below. The instructions or guidance may be visual with text and/or imagery via a graphical user interface (GUI) on the display of the apparatus, and/or audible instructions provided via a speaker of the apparatus, for example. Various GUI screen prompts for the steps in the diagnostic method 750 are described below by way of example only. It will be appreciated that numerous variations in the GUI screen prompts are possible, and the GUI screen prompts may be provided alone or in combination with audio prompts or instructions over a speaker or audio output device.

As will be explained further below, in some configurations, the apparatus may present step-by-step instructions or prompts on how to use the measurement device during the diagnostic mode or diagnostic session. By way of example only, the visual and/or audible instructions or prompts or guidance may provide information to the user about any one or more of the following: information on the operating mode of the apparatus (e.g. therapy or diagnostic mode); information on how to disconnect the patient interface from the apparatus and/or breathing conduit; information on how to connect or attach the measurement device to the gases outlet, breathing conduit or flow path of the apparatus; information on how to perform the forced exhalation manoeuvres; and information on how to perform normal (tidal) breathing at the end of the diagnostic session following the forced exhalation manoeuvres. The information can be provided using any combination of text, numbers, characters, images, icons, graphics, animations, videos and/or audio.

In one typical example, the user may power up or start the breathing assistance apparatus 10 in preparation for a therapy session. At this point, they may have donned the patient interface, e.g. nasal cannula. If it is their first therapy session of the day, or they are otherwise prompted, they may be asked via the GUI on the display of the apparatus 10 to respond to a patient health questionnaire or enquiry. The user will then typically follow the screen prompts and questions presented on the GUI and enter their answers for processing. Examples of a patient health questionnaire or enquiry process that may be carried out is provided in PCT Application Publication No. WO2021/090184, filed 4 Nov. 2020, which is incorporated by reference herein in its entirety. Following completion of the questionnaire, the patient may be prompted or asked to perform a diagnostic test or assessment (e.g. a spirometry test or other lung function or lung performance test for example), and the apparatus will enter the patient diagnostic mode (e.g. spirometry mode or measurement mode) at step 752. In one configuration, the patient diagnostic mode may be triggered or prompted at least partially due to the patient's responses or answers to the questionnaire or health enquiry, but not necessarily. In another configuration, the patient diagnostic mode may be prompted in any manner previously described, regardless of whether a questionnaire or health enquiry is carried out by the user.

### Instruct User to Remove and Disconnect Patient Interface

After initiation of the patient diagnostic mode, the user or patient is instructed at step 754 to remove the patient interface 51 (e.g. nasal cannula or other interface, depending on the therapy) from their face and disconnect it from the end of the breathing conduit 16. For example, the user may have donned the patient interface if they had previously been using the breathing apparatus in therapy mode or if they were about to start a new therapy session, before the patient diagnostic mode had been initiated. Alternatively, the user may simply be prompted to disconnect or remove the patient interface from the end of the breathing conduit 16, which inherently involves or requires removing the patient interface from their face if it had previously been used in a therapy session or had be donned in preparation for a new therapy session. In some situations, the patient diagnostic mode may be entered directly on start-up, and in such situations the patient interface may not have been donned by a user, and therefore the user need only disconnect the patient interface from the end of the breathing conduit 16 or otherwise confirm that the patient interface is not connected to the breathing conduit 16.

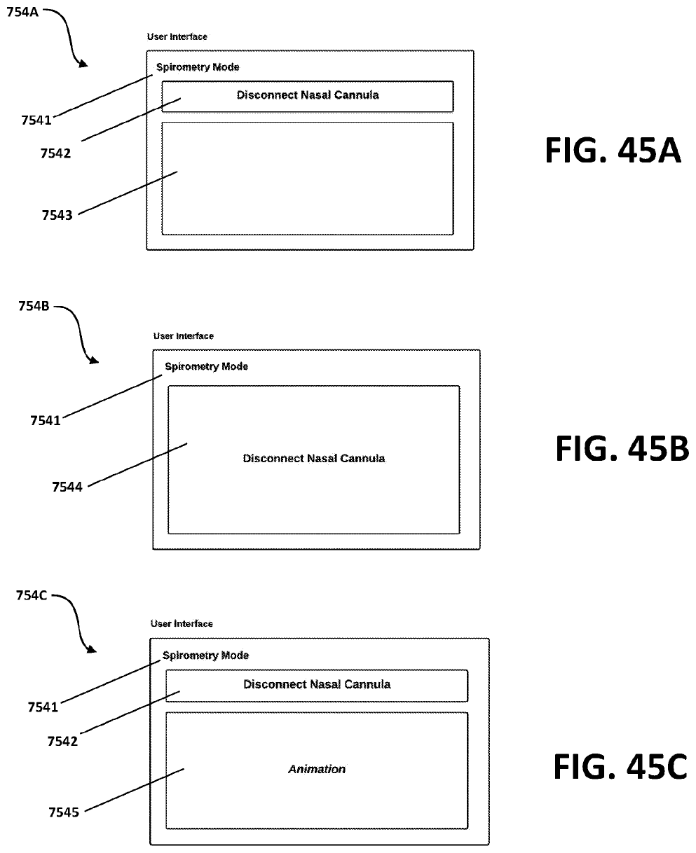

FIGS. 45A-45C show schematic examples of one or more GUI display screen prompts 754A, 754B, 754C that may be displayed on the display of the user interface of the apparatus during step 754. Various different variations are possible, as will be appreciated. Example screen prompt 754A in FIG. 45A includes text and/or graphics field 7541 describing or indicating the current operating mode, i.e. patient diagnostic mode or spirometry mode. A main instruction text and/or graphics field 7542 includes text and/graphics instructing the user to disconnect the patient interface, which in this example is a nasal cannula. One or more secondary or additional text and/or graphics fields 7543 may also be provided that show additional instructions or detail about how to carry out the main instruction, for example. The example screen prompt 754B in FIG. 45B shows a variant in which there is a single main instruction text and/or graphics field 7544. The example screen prompt 754C in FIG. 45C shows a variant in which there is an animation field or region 7545 which may display a 2D or 3D graphic, image, animation, or video depicting or indicative of the instructions, i.e. to assist in guiding the user through the actions required to complete the step or instructions, which in this case is removing or disconnecting the patient interface from the breathing conduit. The GUI display screen prompts may comprise any combination of text, imagery, animations, videos and/or graphics, to provide the user with information and/or guidance on the instructions or prompts for each step.

In one example configuration, the apparatus may additionally execute a breath detection algorithm to confirm or ensure that the patient has removed their patient interface and/or is not connected to the breathing assistance apparatus, before moving to the next step 756 in the process of prompting the user to attach the measurement device 702 to the breathing conduit 16. In this example configuration, the breath detection algorithm may serve as a gate to prevent further steps in the diagnostic method process 750 from being executed until successful disconnection of the patient interface from the breathing conduit is detected or confirmed by the algorithm. An example breathing detection algorithm that may be executed by the apparatus is provided in PCT Application Publication No. WO2020/178746, filed 4 Mar. 2020, which is incorporated by reference herein in its entirety.

### Connect Measurement Device

The next step 756 in the diagnostic method 750 is that the user is prompted to connect or attach the measurement device 702 to the end of the breathing conduit 16 (or other gases outlet along the flow path) of the apparatus 10 in the manner previously described. The user may pre-assemble or install the removable mouthpiece 730 before or after connecting the measurement device 702 to the end of the breathing conduit 16. The apparatus 10 may for example instruct the user to connect the measurement device 702 to the breathing conduit and may provide information or guidance on how to connect the components, in some configurations.

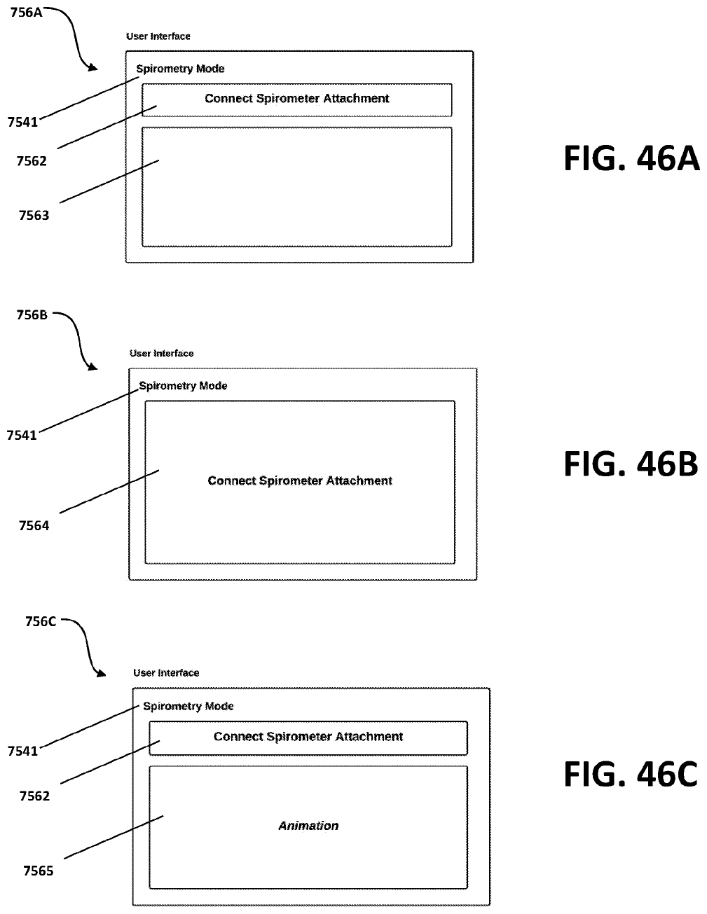

FIGS. 46A-46C show schematic examples of one or more GUI display screen prompts 756A, 756B, 756C that may be displayed on the display of the user interface of the apparatus during step 756. Various different variations are possible, as will be appreciated. Example screen prompt 756A in FIG. 46A includes text and/or graphics field 7541 describing or indicating the current operating mode, i.e. patient diagnostic mode or spirometry mode. A main instruction text and/or graphics field 7562 includes text and/graphics instructing the user to connect or attach the measurement device 702 to the breathing conduit 16. One or more secondary or additional text and/or graphics fields 7563 may also be provided that show additional instructions or detail about how to carry out the main instruction, for example. The example screen prompt 756B in FIG. 46B shows a variant in which there is a single main instruction text and/or graphics field 7564. The example screen prompt 756C in FIG. 46C shows a variant in which there is an animation field or region 7565 which may display a 2D or 3D graphic, image, animation or video depicting or indicative of the instructions, i.e. to assist in guiding the user through the actions required to complete the step or instructions, which in this case is connecting the measurement device to the apparatus.

### Flow of Gases Started to Create Pneumatic Resistance

The next step 758 in the diagnostic process 750 carried out in the patient diagnostic mode is that the controller is configured to control or initiate the flow generator 11 of the breathing assistance apparatus 10 to deliver a flow of gases along the flow path to the gases outlet 21 such that it flows through the breathing conduit and into the measurement device 702. The flow of gases to the measurement device provides a pneumatic resistance for the lung performance measurements or assessments, as previously described.

In this embodiment, the patient diagnostic mode may be configured to provide the flow of gases at a fixed or constant predetermined or configurable flow rate setting, so as to provide a constant or fixed air resistance or pneumatic resistance. The flow of gases may be provided for a predetermined or configurable time period or until the user has completed the required measurements (i.e. performed the one or more lung performance manoeuvres) for a diagnostic session. The flow rate setting may vary depending on the patient and/or particular lung performance measurement or assessment being undertaken, or the flow rate setting may be universally set or configured based on a measurement or industry standard related to the resistance required for particular lung performance measurements or assessments.

In some embodiments, the patient diagnostic mode may be configured or operable to control the composition of gases delivered during the measurement process. In some configurations, the breathing assistance apparatus is controlled to deliver a flow of air or atmospheric air (i.e. if supplementary gases such as oxygen are available, they are turned off or their flow rate reduced to zero via a controllable valve for example). In other configurations, the flow of gases may be air augmented with a supplemental gas such as oxygen and the oxygen fraction of the flow of gases may be controlled to a particular oxygen concentration setting. It will be appreciated that any suitable composition of one or more gases could be delivered.

In some configurations, the breathing assistance apparatus 10 may deliver non-humified flow of gases. For example, the breathing assistance apparatus may not have a humidifier, or the humidifier may be switched off. In other configurations, the flow of gases may be humidified by a humidifier 52 of the breathing assistance apparatus.

### User Instructed to Perform Forced Expiratory Manoeuvres Against Flow of Gases

Once the flow of gases is being delivered to the measurement device 702, the user is then prompted or instructed to perform one or more forced expiratory manoeuvres into the mouthpiece of the measurement device 702, as indicated at step 760. By way of example, the user may be instructed to perform forced blowing or exhaling into the mouthpiece of the measurement device against the pneumatic resistance created by the flow of gases. In one example, the forced exhalations may be similar to those conducted during spirometry assessments and/or measurements.

In one example configuration, the apparatus may prompt the user to exhale for a fixed period, e.g. for a specific time period. The time period may be 1, 2, 3 or more seconds, by way of example only, or some other specified time period depending on the assessment being undertaken. By way of example, the user may be prompted to breath in, and then exhale as hard as they can into the mouthpiece of the measurement device for a specific time period or until they are out of breath. A countdown timer may be displayed to the user to show how long they need to exhale into the measurement device for.

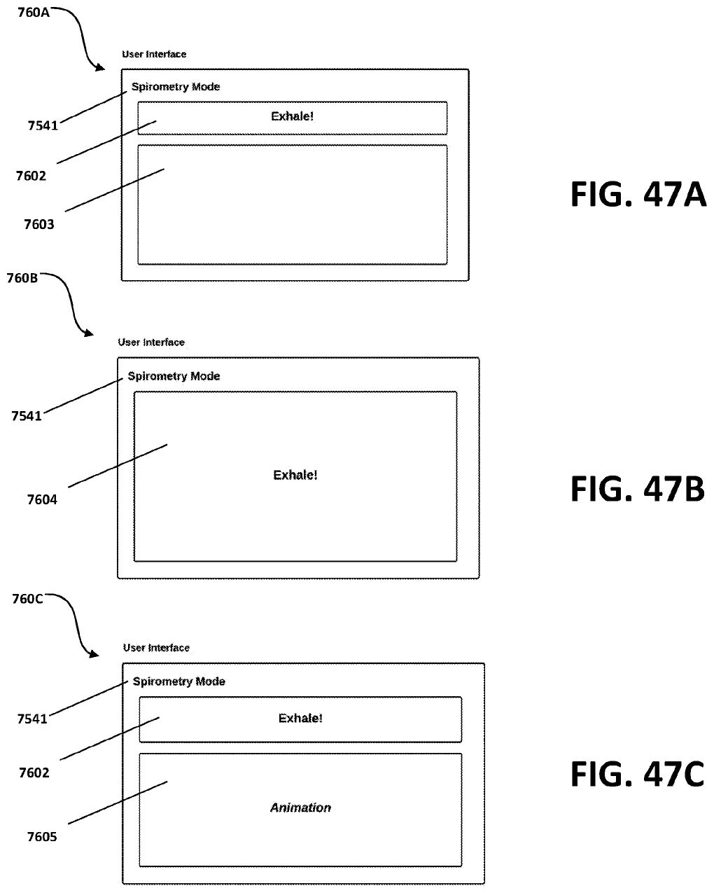

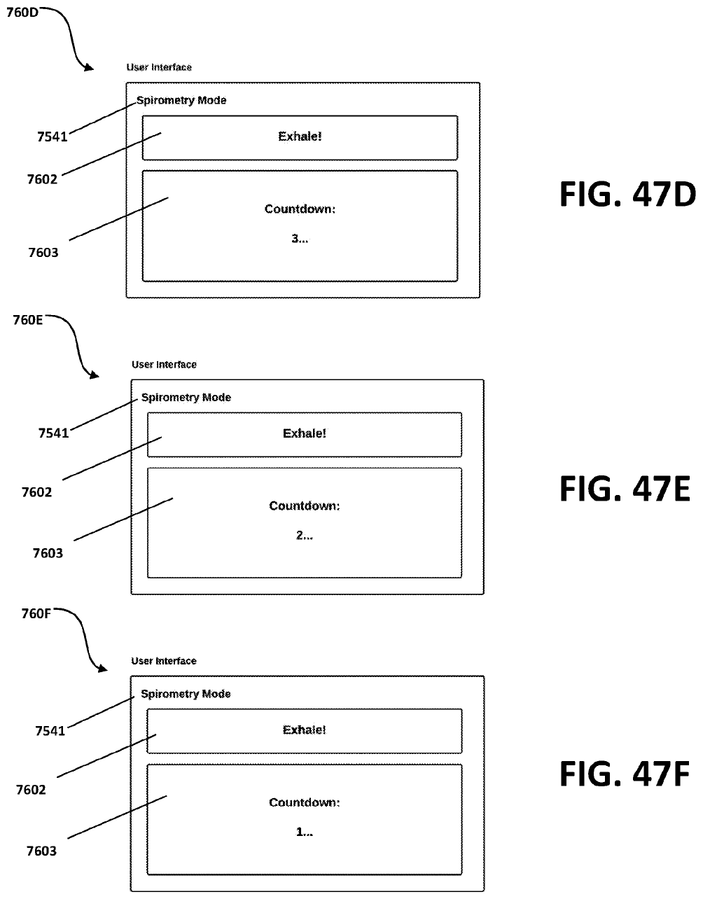

FIGS. 47A-47F show schematic examples of one or more GUI display screen prompts 760A, 760B, 760C, 760D, 760E, 760F that may be displayed on the display of the user interface of the apparatus during step 760. Various different variations are possible, as will be appreciated. Example screen prompt 760A in FIG. 47A includes text and/or graphics field 7541 describing or indicating the current operating mode, i.e. patient diagnostic mode or spirometry mode. A main instruction text and/or graphics field 7602 includes text and/graphics instructing the user to perform a forced expiratory or exhalation manoeuvre into the measurement device 702 against the flow of gases. One or more secondary or additional text and/or graphics fields 7603 may also be provided that show additional instructions or detail about how to carry out the main instruction, for example. The example screen prompt 760B in FIG. 47B shows a variant in which there is a single main instruction text and/or graphics field 7604. The example screen prompt 760C in FIG. 47C shows a variant in which there is an animation field or region 7605 which may display a 2D or 3D graphic, image, animation, or video depicting or indicative of the instructions, i.e. to assist in guiding the user through the actions required to complete the step or instructions, which in this case is performing one or more expiratory or exhalation manoeuvres.

Example screen prompts 760D, 760E, 760F depict a sequence of screen prompts in the format of the screen prompt 760A, in which the secondary or additional text and/or graphics region 7603 comprises a countdown timer. The countdown timer may provide guidance to the user as to how long they need to perform a forced exhalation manoeuvre for, in some configurations. In this example, the countdown is provided with text and numerals, but it will be appreciated that a countdown timer may be provided in animation or graphics or videos either alone or in combination with text and numerical information.

Sensor Data Collected, Stored and/or Analysed

As the user or patient performs the expiratory manoeuvre or manoeuvres, the patient diagnostic mode is configured to sense or measure one or more characteristics of the flow of gases via one or more sensors of the breathing assistance apparatus, as indicated at step 762. In particular, the controller receives or retrieves sensor data from one or more sensors during the expiratory manoeuvres carried out by the patient.

In one example embodiment, the controller of the breathing assistance apparatus is configured to sample the flow rate signal of one or more flow rate sensors of the apparatus. As discussed, the apparatus may comprise one or more flow rate sensors or configurations that are arranged to sense the flow rate of the flow of gases and generate a representative flow rate signal or flow rate data. In other embodiments, one or more other characteristics of the flow of gases may be sensed and measured during the expiratory manoeuvres including, but not limited to, pressure, temperature, humidity, gas concentration, or any other property that may be useful directly or indirectly for analysing lung performance.

### Repeated Expiratory Manoeuvres

In some configurations, the apparatus may be configured, while in the patient diagnostic mode, to instruct the user to perform repeated or multiple expiratory manoeuvres in a diagnostic session. In one example, the user may be instructed to perform a plurality or multiple forced exhalations, spaced apart by a predetermined time interval. For example, three or some other specified number of forced exhalations might be performed in a uniformly or non-uniformly spaced-apart manner or according to a preconfigured time interval or time format, and sensor data for each exhalation manoeuvre is extracted or stored from the one or more relevant sensors to create multiple sets of exhalation measurement data, one set for each exhalation manoeuvre.

In some configurations, the apparatus may be configured to detect or identify the onset or start of a forced exhalation by the patient by processing and/or monitoring the sensor data. For example, the apparatus may be configured to process the flow rate signal or flow rate data received during the diagnostic mode to identify an offset in the flow rate relative to a threshold or range, or identify a significant deviation that is otherwise indicative of a user commencing a forced exhalation manoeuvre into the measurement device 702 against the flow of gases. Upon detecting the onset or start of the forced exhalation manoeuvre, the apparatus may trigger the countdown timer and/or other display GUI screen prompts or other instructions to request the user to continue the forced exhalation for the required each manoeuvre time period.

Additionally or alternatively, the forced exhalation detection or identification can be used to count the number of repeated exhalation manoeuvres that have been performed by the user in the diagnostic session. The manoeuvre count can be compared against the minimum number required or other threshold, and can be used to prompt the user to continue doing forced exhalation manoeuvres until the required number have been registered. Each forced exhalation manoeuvre will have its own respective set of associated measurement data (e.g. sensor data) gathered from the one or more sensors, for subsequent processing.

The sets of exhalation measurement data from each manoeuvre may then be combined or aggregated or otherwise processed to create an averaged set of data. Alternatively, other statistical analyses may be applied to the measurement data to extract a filtered or robust set of data with reduced noise or which is less impacted by anomalies, such as the user making a mistake in following the instructions or performing the manoeuvres correctly. In some configurations, the highest quality or best set of measurement data may be selected from the sets of exhalation measurement data for subsequent processing and analysis for lung performance. For example, if the user is required to carry out at least three separate exhalation manoeuvres, the sets of sensor data from each exhalation manoeuvre may be processed relative to each other to select the best or highest quality of the three or more sets of data from the diagnostic. The highest quality set of measurement data may be selected based on one or more criteria such as, but not limited to, those already mentioned.

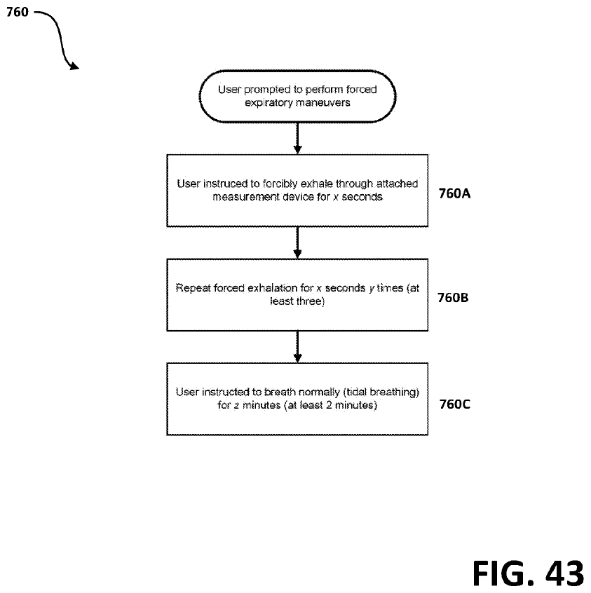

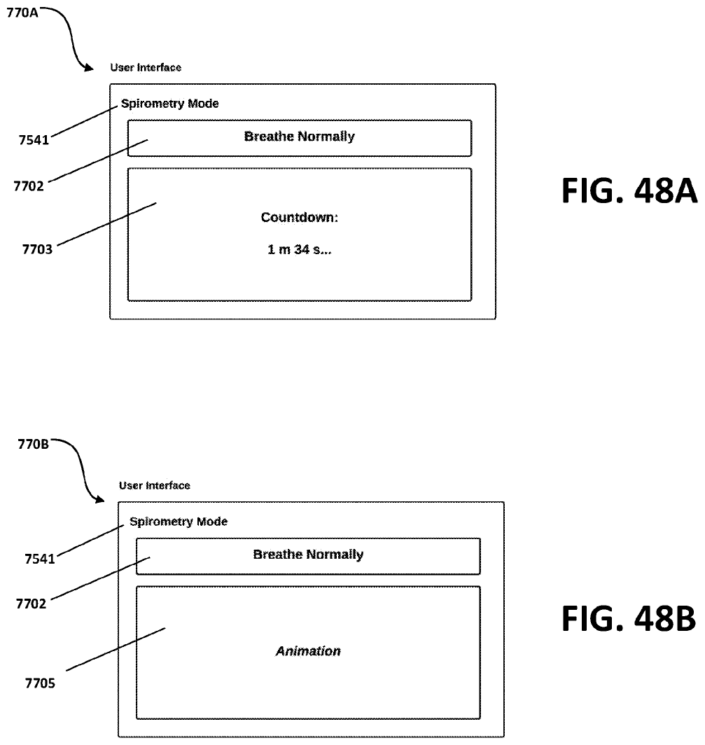

Referring to FIGS. 43, 48A, and 48B, a more detailed explanation of one example of the process of prompting the user to perform forced expiratory manoeuvres at step 760 in the diagnostic method 750 will be provided.

In this example configuration, the user is instructed to forcible exhale through or into the mouthpiece of the measurement device 702 for x seconds, as shown at sub-step 760A.

The user is then instructed to repeat the forced exhalation for x seconds y times, as shown at sub-step 706B. The variables x and y may be configured or set as desired. In one example, the apparatus is configured so that the user is prompted to undertake a minimum number of forced exhalation manoeuvres in the diagnostic session, e.g. at least three or some other suitable number.

After the user has performed the required number of repeated forced exhalation manoeuvres (which may be optionally automatically detected by the apparatus as described above or otherwise confirmed by user input), the user is prompted to breath normally for a specific time period, as shown at sub-step 760C to complete or end the diagnostic session. For example, the user may be prompted to undertake normal or tidal breathing for z minutes (e.g. at least 2 minutes or some other time period). It will be appreciated that the variable z may be configured or altered as desired. In one embodiment, during this end breathing session 760C, sensor data may be recorded and stored. The stored sensor data for the normal breathing may then be further processed and/or used to extract or calculate one or more tidal breathing measures or parameters for the patient, as explained further below.

FIGS. 48A and 48B show schematic examples of one or more GUI display screen prompts 770A and 770B that may be displayed on the display of the user interface during sub-step 760C. Various different variations are possible, as will be appreciated. Example screen prompt 770A in FIG. 48A includes text and/or graphics field 7541 describing or indicating the current operating mode, i.e. patient diagnostic mode or spirometry mode. A main instruction text and/or graphics field 7702 includes text and/graphics instructing the user to breath normally. One or more secondary or additional text and/or graphics fields 7703 may also be provided that show additional instructions or detail about how to carry out the main instruction, for example. In this example, the display region 7703 may comprise a countdown timer which displays how long the user should continue to breath normally, before the diagnostic session ends. The example screen prompt 770B in FIG. 48B shows a variant in which there is an animation field or region 7705 which may display a 2D or 3D graphic, image, animation or video depicting or indicative of the instructions, i.e. to assist in guiding the user through the actions required to complete the step or instructions, which in this case is breathing normally for a preset or specified period of time.

Lung Performance Assessment from Sensor Data

One or more processing algorithms may be applied to the sensor or measured data from the expiratory manoeuvres to extract, identify or analyse one or more features indicative of lung performance. In one example, lung performance measurements or data analogous to or indicative of spirometry measurements or data may be extracted from or identified in the sensor data.

By way of example, in one configuration the sensed flow rate data of the flow of gases in the flow path of the breathing assistance apparatus is collected and stored during the expiratory manoeuvre or manoeuvres. This flow rate data may represent measurement data indicative of lung performance. The flow rate data (sensed in the breathing assistance apparatus) fluctuates as the user performs a forced expiration through or into the measurement device, which is in fluid communication with the flow path of the breathing assistance apparatus. The fluctuation in the flow rate signal or data enables one or more features indicative of lung performance to be identified and extracted from the flow rate signal, via further processing of the set of flow rate data (e.g. sensor data) associated with the expiratory manoeuvre.

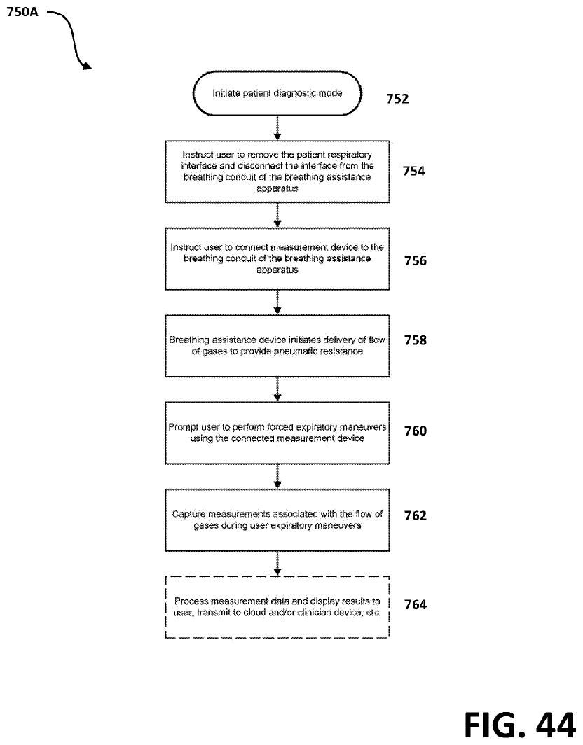

Referring to FIG. 44, another example diagnostic method or process 750A is shown which is a modified version of the diagnostic method 750 shown in FIG. 42. Like reference numerals represent like steps. The modified diagnostic method 750A includes an optional additional step 764 which relates to any one or more of the following: processing and/or analysing the measurement data (sensor data) gathered during the forced exhalation manoeuvres to generate lung performance results or result data, storing the measurement data and/or results, transmitting the measurement data and/or results, and/or displaying the measurement data and/or results. By way of example, the sensor data or measurement data may be stored and processed by the controller of the breathing assistance apparatus, and/or transmitted to an external device or server or system for storage and/or further processing and/or extraction of lung performance parameter or measurements for the user. The sensor data and/or lung performance measurements generated, whether represented graphically, numerically or in any other format, may be displayed to the user on the display of the breathing assistance apparatus and/or transmitted to one or more external or remote devices, systems or servers (e.g. Cloud platforms), for storage, access, display and/or viewing by a clinician or other authorised persons for example.

In one configuration, the raw measurement or sensor data may be processed by the controller of the breathing assistance apparatus to generate the one or more lung performance measurements or data.

In another configuration, the raw measurement or sensor data may be transmitted to an external or remote electronic device (e.g. smart phone, tablet, PC, wearable, remote server, remote system, Cloud, or other processing device) for processing to generate the one or more lung performance measurements or data. The raw measurement or sensor data may be transmitted in real-time during the diagnostic mode or at the end or conclusion of the diagnostic mode.

In another configuration, the raw measurement or sensor data may be partly processed by the controller of the breathing assistance apparatus and partly processed by an external or remote electronic device (e.g. smart phone, tablet, PC, wearable, remote server, remote system, Cloud, or other processing device).

In configurations where the raw or partly processed measurement or sensor data is processed by an external or remote electronic device (e.g. smart phone, tablet, PC, wearable, remote server, remote system, Cloud, or other processing device), the external or remote device or system may transmit the processed data (e.g. lung performance measurements or data) back to the breathing assistance apparatus or another device for storage and/or display, and/or retain or store the processed data, and/or perform further actions or processing including displaying the processed data or results on an associated display screen.

In one example configuration, the controller of the breathing assistance apparatus may be configured to transmit the measurement or sensor data to a user's electronic device (e.g. a smart phone or tablet or similar). The user's electronic device may perform processing of the raw measurement or sensor data to generate the lung performance measurements or data, and may then transmit that processed data to a remote server or system for further processing, storage and/or display. In some configurations, the breathing assistance apparatus may only have short-range data communication capability (e.g. Bluetooth, NFC, infrared, wired) with the user's electronic device (e.g. smartphone or tablet or similar). The user's electronic device may have additional longer-range data communication capabilities (e.g. Wifi, cellular, 4G, LTE, 5G, LAN) and may then transmit or send the raw or processed data onto a remote server or system, thereby acting as a relay or transmitter for the breathing assistance apparatus or a data communication bridge between the breathing assistance apparatus and remote server or system.

### Diagnostic Process Complete-Switching to Therapy Mode

In some configurations, at the end of the diagnostic method or processes 750,750A, the apparatus may switch to another mode or back to a normal mode, e.g. a therapy mode, either automatically or in response to manual input or control by the user. For example, the apparatus may switch to the therapy mode after sufficient measurement data from the diagnostic session has been captured for processing, or after the measurement data has been processed into result data for display, storage, and/or transmission.

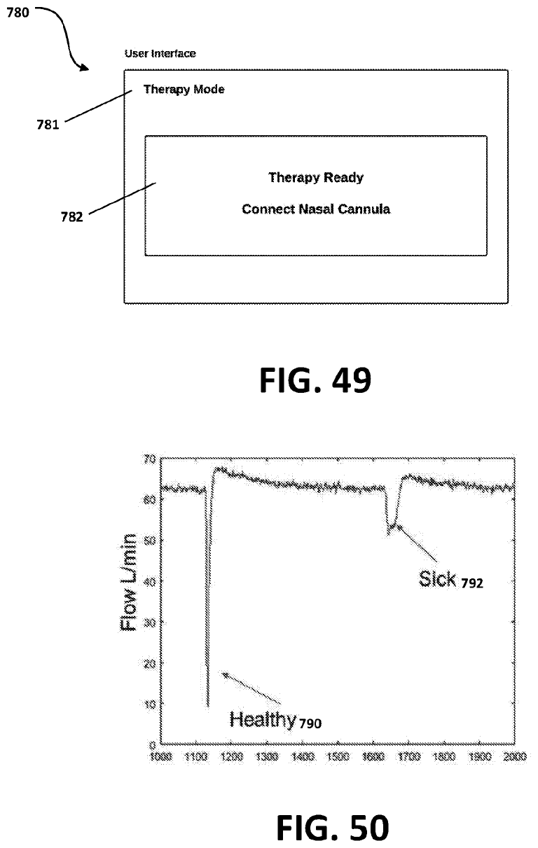

In one example configuration, the user may be prompted or notified on the GUI or display screen that the apparatus has switched to therapy mode or is no longer in diagnostic mode. FIG. 49 shows one schematic example of a GUI display screen prompt 780 that may be displayed on the display of the user interface of the apparatus. Various different variations are possible, as will be appreciated. Example screen prompt 780 in FIG. 49 includes text and/or graphics field 781 describing or indicating that the apparatus has switched to a therapy mode ready for a new therapy session (i.e. for delivery a flow of gases for respiratory therapy). A main instruction text and/or graphics field 782 may also be provided, and may include text and/graphics informing the user about the status of the apparatus and/or instructions on initiating therapy. For example, the display region or field 782 may indicate that the apparatus is ready for therapy to be started and/or may prompt the user to disconnect the measurement device 702 from the apparatus and/or breathing conduit, and to connect or re-connect the patient interface to the breathing conduit. Once the patient interface is connected to the breathing conduit, the user may then don the patient interface and commence the therapy session with the apparatus.

### Example of Lung Performance Feature Extraction

Referring to FIG. 50, an example of the flow rate data signal and the fluctuations caused by forced expiratory manoeuvres into the measurement device is shown. In this example, the flow of gases was set to provide pneumatic resistance at a flow rate of 70 L/min. It will be appreciated that other non-zero flow rates or ranges of flow rates may be suitable for generating suitable measurement data during use of the measurement device with the breathing assistance apparatus in diagnostic mode.

In this example configuration, the flow rate of approximately 70 L/min may provide an advantage of avoiding any back flow of the user's exhaled breath into the apparatus 10. It will be appreciated that other flow rates may also provide this benefit in different configurations, and the flow rate selected may be based on balancing one or more factors and/or to suit particular criteria or a particular configuration of the breathing assistance apparatus and/or measurement device. For example, in some configurations, the flow rate may be configured to provide sufficient pneumatic resistance for performing the required lung performance measurements, and to avoid back flow into the apparatus.

The flow rate selected for use may additionally or alternatively be a function of or based at least partly on the pneumatic properties (e.g. including flow resistance of the flow path or output flow path after the flow generator) of the configuration of the breathing assistance apparatus and/or measurement device. For example, the flow rate may be configured or selected at least partly based on the pneumatic properties of one or more of the following: the output flow path of the breathing assistance apparatus, the breathing conduit, and/or measurement device. For example, in a breathing assistance apparatus with a non-return valve in the flow path, a much lower flow rate (e.g. approximately 10 L/min) may be suitable or sufficient for gathering the required measurement data when running in diagnostic mode with the measurement device attached.

An example of the fluctuation in the sensed flow rate data caused by a healthy person doing a forced exhalation into the measurement device is shown at 790, and the fluctuation in the sensed flow rate data for a sick person is shown at 792. As illustrated in FIG. 50, the healthy person causes a larger and sharper drop in the sensed flow rate of the flow of gases, compared to the sick person or person with reduced or impaired lung function or performance. In one example configuration, the sensor data, e.g. the flow rate sensor data may represent the lung performance measurement data and/or may be processed to extract one or more lung performance measurement data values. In one example configuration, the sensor data (e.g. flow rate or other sensor data) and/or the processed lung performance measurement data may be graphed or represented graphically for display on a user interface. In such configurations, the graphs may be further processed to identify and/or determined the health status of a user, e.g. whether they are healthy or sick.

Examples of lung performance measurements that may be extracted or identified from the measurement data (e.g. sensor data, such as the flow rate signal or flow rate data) from the diagnostic session are further explained below.

The pressure Pₚₐₜᵢₑₙₜ exerted by a patient during the exhalation manoeuvres may be computed from the flow rate signal, for example by applying the following equation:

$${P}_{patient} = {P}_{b l o w e r} - R * {Q}^{{n}_{1}} \qquad ( 1 )$$

Where P_{blower} is the pressure measured at the output of the breathing assistance apparatus using a pressure sensor located in the flow path downstream of the flow generator, R is the resistance to flow between the flow generator output and the patient (including the resistance due to the connected measurement device), Q or Q_{Total} is the flow generator output flow rate (e.g. 70 L/min in the example above with reference to FIG. 50 but could be any other suitable flow rate), and n₁ preferably has a value of 2, but any value between 1 and 2 may be suitable. The value of n₁ may be pre-determined and is a quantifier of how laminar or turbulent the flow through the measurement device attachment is.

The conductance to flow (C) between the flow generator output and the patient (including the effect of the connected measurement device) can be represented as:

$$C = \frac{\int {Q}_{T o t a l}}{\int {P}_{Patient}^{\frac{1}{n}}} \qquad ( 2 )$$

Using the patient pressure value Pₚₐₜᵢₑₙₜ, the flow Qₚₐₜᵢₑₙₜ that the patient is able to induce against the breathing assistance apparatus can be computed as follows:

$${Q}_{Patient} = sign ( {P}_{Patient} ) {\vert \frac{C * {P}_{P a t i e n t}}{{R}_{P a t i e n t}} \vert}^{( \frac{1}{{n}_{2}} )} \qquad ( 3 )$$

Where Rₚₐₜᵢₑₙₜ is the known flow resistance of the measurement device at the flow rate Q (i.e., the flow resistance between the connector end of the measurement device and the mouthpiece of the measurement device), and again n₂ has a value of 2, but any value between 1 and 2 may be suitable.

An alternative approach or embodiment for extracting the Qₚₐₜᵢₑₙₜ signal may also be implemented, as will described with reference to equations (4)-(6) below. First, the amount of leak flow (Q_{Leak}) through the exhaust vent(s) or openings of the measurement device can be estimated as:

$${Q}_{L e a k} = C * {P}_{{Patient}^{}}^{\frac{1}{n}} \qquad ( 4 )$$

Where n has the same definition as the previous components n₁ and n₂. Given that Q_{Total} can be measured directly using one or more flow sensors downstream of the flow generator/blower, the controller is able to estimate the Q_{Patient} signal:

$${Q}_{Patient} = {Q}_{Total} - {Q}_{Leak} \qquad ( 5 )$$

Because the value of Q_{Leak} is particularly significant in this alternative approach, it may be important to detect if there is a leak flow at all (i.e., determine if the exhaust vent or vents of the measurement device covered/occluded) in some configurations. This could be achieved through a check such as:

$$\frac{{Q}_{T o t a l}}{{P}_{{}^{{}^{{}^{{Patient}^{}}}}}^{\frac{1}{n}}} > {C}_{T h r e s hold} \qquad ( 6 )$$

Where the threshold conductance, C_{Threshold}, has a pre-defined value. For example, 83.2 L/min/cmH₂O^{1/n}. The threshold may have to be satisfied for a minimum period of time (e.g., in seconds), to ensure accuracy.

Several clinically useful lung performance metrics, e.g. features or feature indicators and analogues, may be extracted from the sensed data generated during the diagnosis mode. These may include FEV₁, FEV₂, FEV₃ (Fractional Expiration Volume in 1, 2, 3 seconds respectively), FVC (Forced Vital Capacity), PEF (Peak Expiratory Flow), or others.
An FEV₁ value or FEV₁ analogue may be computed by analysing the first second of flow rate data once a patient has begun exhaling in an exhalation manoeuvre.
Similarly, FEV₂ and FEV₃ values or analogues may be computed by analysing the first two and three seconds (respectively) of flow rate data once a patient has begun exhaling in an exhalation manoeuvre.
FVC may be computed by determining the total volume of air exhaled over a full exhalation manoeuvre. This may, for example, involve integrating the processed patient flow rate signal (Qₚₐₜᵢₑₙₜ) between the start and finish of an exhalation. Alternatively, the patient flow rate signal may instead be represented by negative fluctuations in the unprocessed sensed raw flow rate signal, and the FVC may be computed from these negative fluctuations.
An FEV₁/FVC ratio—another lung performance metric—may be readily computed from the FEV₁ and FVC values described above.
PEF may be computed by identifying a peak patient flow, corresponding to the highest peak in the processed patient flow rate signal Qₚₐₜᵢₑₙₜ or alternatively the deepest ‘trough’ in the raw flow rate signal, as seen in FIG. 50.

Although the foregoing disclosure has described forced exhalation measurements, some measurements can be computed from Q_{Patient} signal(s) generated during restful breathing or tidal breathing by the patient into the measurement device. In this tidal breathing mode, the flow generator output flow rate Q may be lower compared to the forced exhalation measurement mode, and may be 10 L/min or some other suitable flow rate for example.

Examples of other measurements that can be extracted or determined during the tidal (restful) breathing mode into the measurement device may include any one or more of the following:
Respiratory rate (RR) may be computed via zero-crossing detection, peak detection, frequency analysis, or other suitable means of identifying the time period of processed patient flow rate signal Q_{Patient}.
Tidal volume (VT) may be computed by integrating the Q_{Patient} signal over one or more breathing cycles contained in the signal.
Minute ventilation (MV) may be computed using the RR and VT parameters above, i.e.,

$$M V = \frac{{V}_{T}}{R R} .$$

The inspiratory time to breath time ratio (Ti/Ttot) may be computed by identifying the time periods of inspiration and total breath time via zeros crossings or some other suitable means of identifying the time period of processed patient flow rate signal Q_{Patient}.

Any of these lung performance metrics or features, individually or in combination, are particularly useful for analysing the status or symptoms of a patient with COPD, asthma, bronchiectasis, or other respiratory condition that affects lung health or performance. One or more of these features, or other suitable lung performance parameters may be used to help guide selection of suitable therapy settings (e.g. a prescription for respiratory therapy) for a breathing assistance apparatus (e.g. a respiratory therapy device). For example, FiO₂ and/or flow rate settings may be at least partially informed by one or more lung performance parameters determined using the measurement device connected to the breathing assistance apparatus operating in a diagnostic mode. Upper and/or lower boundaries for said therapy settings or prescription may also be selected based at least partly on the lung performance parameters or metrics generated during the diagnosis mode.

In one example configuration, a clinician may receive and review the lung performance metrics, measurements, results or features generated, and then propose a prescription to the patient at least partly based on the measurement results. The prescription may define or comprise any one or more of the following settings or characteristics for the flow of gases provided in the respiratory therapy: flow rate, oxygen concentration, and humidity level.

### Representation of Lung Performance Measurements

Any one or more of the lung performance measurements, features, values or indicators in the result data that are extracted, identified, or calculated from a diagnostic or diagnosis session with the measurement device may be represented, stored, recorded or displayed on their own or as a ratio, percentage, or fraction relative to the expected value of a healthy member of the population group the user belongs to or relative to some other baseline value or parameter.

For example, PEF may be represented as a ‘PEF as a percentage of a healthy adult male or female’. The indicator values expected for a healthy adult male or female can be stored in look-up tables or other suitable data structures which may be stored in memory of the apparatus or other accessible remote memory or data storage (e.g. Cloud or remote server data storage).

In a further example, any one or more of the individual lung performance measurements, indicators, features, or values in the result data may be combined in any one or more desirable ratios or functions relative to each other to generate new useful lung performance metrics or ratios. For example, calculating the ratio or value of FEV1/FVC may be useful as a spirometry assessment. As an example, FEV1/FVC may desirably be between 70-80% (or 0.7-0.8) to indicate good health.

### Patient Physiological Parameters

In an example configuration, if the patient is instructed or prompted to take multiple breaths at the end of a diagnostic period (e.g. see sub-step 760C above in FIG. 43), one or more patient physiological parameters may be estimated or calculated or extracted from the sensor data recorded over the multiple breath manoeuvres. These parameters may include, for example, any one or more of the following: tidal volume, respiratory rate, minute ventilation, and peak inspiratory flow.

### 4. Terminology

The phrase ‘breathing assistance apparatus’ as used in the specification and claims is intended to mean, unless the context suggests otherwise, any type of breathing assistance or respiratory apparatus, device, or system, that is operable to provide respiratory support or respiratory therapy to a user or patient by providing a flow of gases to the user or patient.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1.-99. (canceled)

100. An apparatus for performing lung performance measurements on a user, comprising:
a flow generator that is operable to generate a flow of gases in a flow path;
a measurement device that is in fluid communication with the flow path to receive the flow of gases;
a controller that is operable to control the flow generator to generate a flow of gases into the measurement device to provide a controlled pneumatic resistance in the flow path to user exhaled breath as the user performs one or more forced expiratory maneuvers into the measurement device; and
one or more sensors that are configured to sense one or more characteristics of the flow of gases in the flow path as the user performs the forced expiratory maneuvers into the measurement device and generates representative sensor data from which one or more lung performance measurements can be obtained.

101. The apparatus of claim 100, wherein the controller is configured to:
determine a user flow rate signal representing the flow the user is able to induce against the flow of gases via the measurement device based on a function of at least a user pressure signal representing the pressure exerted by the user during exhalation into the measurement device; and/or
determine a user flow rate signal representing the flow the user is able to induce against the flow of gases via the measurement device based on a function of at least a flow resistance parameter relating to the measurement device; and/or
determine a user flow rate signal representing the flow the user is able to induce against the flow of gases via the measurement device based on a function of at least a flow conductance parameter; and/or
determine a user flow rate signal representing the flow the user is able to induce against the flow of gases via the measurement device based on a function of at least a flow rate signal representing the flow rate of the flow of gases generated by the flow generator and a leak flow signal representing the leak flow through one or more exhaust openings of the measurement device.

102. The apparatus of claim 101, wherein the controller is configured to determine the leak flow signal based on a function of at least a flow conductance parameter and a user pressure signal representing the pressure exerted by the user during exhalation into the measurement device.

103. The apparatus of claim 101, wherein the controller is configured to determine the user pressure signal based on a function of at least a pressure signal representing the pressure at an outlet of the flow generator, a resistance to flow parameter, and a flow rate signal representing the flow rate of the flow of gases generated by the flow generator.

104. The apparatus of claim 100, wherein the controller is configured to determine a tidal volume signal or parameter for the user based at least partly on or as a function of at least a determined user flow rate signal representing the flow the user is able to induce against the flow of gases via the measurement device, as the user performs tidal breathing into the measurement device.

105. The apparatus of claim 104, wherein the controller is configured to determine a respiratory rate signal or parameter for the user based at least partly on or as a function of at least the determined user flow rate signal, as the user performs tidal breathing into the measurement device, and wherein the controller is configured to determine a minute ventilation signal or parameter for the user based at least partly on or as a function of at least the determined respiratory rate signal and the determined tidal volume signal.

106. The apparatus of claim 101, wherein the controller is configured to determine a signal or parameter representing an inspiratory time to total breath time ratio based at least partly on or as a function of at least the determined user flow rate signal, as the user performs tidal breathing into the measurement device.

107. The apparatus of claim 100, wherein the one or more sensors are located in the flow path upstream of the measurement device or configured to sense the flow of gases upstream of the measurement device; and/or wherein the flow generator is enclosed or contained in a main housing of the apparatus, and wherein the one or more sensors are located within the main housing of the apparatus.

108. The apparatus of claim 100, wherein the apparatus further comprises a humidifier that is operable to heat and humidify the flow of gases generated by the flow generator; and optionally wherein the humidifier is provided in a main housing of the apparatus, the main housing further comprising the flow generator.

109. The apparatus of claim 100, wherein the controller comprises a patient diagnostic mode that is operable to control the flow of gases to provide the controlled pneumatic resistance when the measurement device is connected to the flow path.

110. The apparatus of claim 100, wherein the controller comprises one or more operating modes, including at least a first mode that is operable to control the apparatus when the measurement device is not connected to the flow path and a second mode that is operable to control the apparatus when the measurement device is connected to the flow path; and/or wherein the controller comprises a plurality of operating modes, including at least a therapy mode that is operable to control the apparatus to provide the flow of gases to user for respiratory therapy, and a patient diagnosis mode that is operable to control the flow of gases to provide a pneumatic resistance for performing lung performance measurements when the measurement device is connected to the flow path.

111. The apparatus of claim 100, wherein the apparatus further comprises a gases outlet for the flow of gases, and wherein the measurement device is a component or attachment that is directly connected to the gases outlet, or indirectly connected to the gases outlet via a breathing conduit.

112. The apparatus of claim 100, wherein the measurement device comprises a main body extending between a connector end and a mouthpiece end, the main body comprising a main lumen or lumens extending between the connector end and mouthpiece end for the flow of gases; and optionally wherein the main body is a conduit or tubular component with an opening or openings at each end.

113. The apparatus of claim 112, wherein the connector end of the main body is configured to be releasably connected to an end of a breathing conduit, the breathing conduit providing delivery of the flow of gases to a user; and/or wherein the connector end of the main body is integrally or permanently connected to an end of a breathing conduit, the breathing conduit providing delivery of the flow of gases to a user; and/or wherein the connector end of the main body comprises one or more openings or ports for fluidly connecting to an end of a breathing conduit, the breathing conduit providing delivery of the flow of gases to a user.

114. The apparatus of claim 112, wherein the main body of the measurement device further comprises one or more exhaust openings that are in fluid communication with the main lumen or lumens.

115. An apparatus for performing lung performance measurements on a user, comprising:
a flow generator that is operable to generate a flow of gases in a flow path;
a measurement device that is in fluid communication with the flow path to receive the flow of gases;
a controller that is operable to control the flow generator to generate a flow of gases into the measurement device to provide a controlled pneumatic resistance in the flow path to user exhaled breath as the user performs restful or tidal breathing into the measurement device; and
one or more sensors that are configured to sense one or more characteristics of the flow of gases in the flow path as the user performs the restful or tidal breathing into the measurement device and generates representative sensor data from which one or more lung performance measurements can be obtained.

116. The apparatus of claim 115, wherein the controller is configured to:
determine a user flow rate signal representing the flow the user is able to induce against the flow of gases via the measurement device based on a function of at least a user pressure signal representing the pressure exerted by the user during exhalation into the measurement device; and/or
determine a user flow rate signal representing the flow the user is able to induce against the flow of gases via the measurement device based on a function of at least a flow resistance parameter relating to the measurement device; and/or
determine a user flow rate signal representing the flow the user is able to induce against the flow of gases via the measurement device based on a function of at least a flow conductance parameter; and/or
determine a user flow rate signal representing the flow the user is able to induce against the flow of gases via the measurement device based on a function of at least a flow rate signal representing the flow rate of the flow of gases generated by the flow generator and a leak flow signal representing the leak flow through one or more exhaust openings of the measurement device.

117. The apparatus of claim 116, wherein the controller is configured to determine the flow conductance parameter based on a function of at least a flow rate signal representing the flow rate of the flow of gases generated by the flow generator and a user pressure signal representing the pressure exerted by the user during exhalation into the measurement device.

118. The apparatus of claim 116, wherein the controller is configured to determine a respiratory rate signal or parameter for the user based at least partly on or as a function of at least the determined user flow rate signal, as the user performs tidal breathing into the measurement device.

119. The apparatus of claim 115, wherein the controller is configured to determine a tidal volume signal or parameter for the user based at least partly on or as a function of at least a determined user flow rate signal representing the flow the user is able to induce against the flow of gases via the measurement device, as the user performs tidal breathing into the measurement device.

120. The apparatus of claim 119, wherein the controller is configured to determine a respiratory rate signal or parameter for the user based at least partly on or as a function of at least the determined user flow rate signal, as the user performs tidal breathing into the measurement device, and wherein the controller is configured to determine a minute ventilation signal or parameter for the user based at least partly on or as a function of at least the determined respiratory rate signal and the determined tidal volume signal.

121. The apparatus of claim 115, wherein the measurement device comprises a main body extending between a connector end and a mouthpiece end, the main body comprising a main lumen or lumens extending between the connector end and mouthpiece end for the flow of gases; and optionally wherein the main body is a conduit or tubular component with an opening or openings at each end.
